SEQUENCE LISTING (13 sequences)

SEQ ID NO: 1 - protein, 5 aa, Artificial Sequence
  source: /note=“Description of Artificial Sequence Synthetic peptide”
SYVMH

SEQ ID NO: 2 - protein, 6 aa, Artificial Sequence
  source: /note=“Description of Artificial Sequence Synthetic peptide”
NPYNDG

SEQ ID NO: 3 - protein, 12 aa, Artificial Sequence
  source: /note=“Description of Artificial Sequence Synthetic peptide”
GTYYYGTRVFDY

SEQ ID NO: 4 - protein, 16 aa, Artificial Sequence
  source: /note=“Description of Artificial Sequence Synthetic peptide”
RSSKSLQNVNGNTYLY

SEQ ID NO: 5 - protein, 7 aa, Artificial Sequence
  source: /note=“Description of Artificial Sequence Synthetic peptide”
RMSNLNS

SEQ ID NO: 6 - protein, 9 aa, Artificial Sequence
  source: /note=“Description of Artificial Sequence Synthetic peptide”
MQHLEYPIT

SEQ ID NO: 7 - protein, 121 aa, Artificial Sequence
  source: /note=“Description of Artificial Sequence Synthetic polypeptide”
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 8 - protein, 112 aa, Artificial Sequence
  source: /note=“Description of Artificial Sequence Synthetic polypeptide”
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 9 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 107 aa, Artificial Sequence [sequence omitted]

SEQ ID NO: 11 - protein, 451 aa, Artificial Sequence
  source: /note=“Description of Artificial Sequence Synthetic polypeptide”
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 219 aa, Artificial Sequence
  source: /note=“Description of Artificial Sequence Synthetic polypeptide”
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 556 aa, Homo sapiens [sequence omitted]

(19) United States
(12) Patent Application Publication
(10) Pub. No.: US 2022/0184208 A1
(43) Pub. Date: Jun. 16, 2022

(54) ANTI-CD19 COMBINATION THERAPY

(71) Applicants: MorphoSys AG, Planegg (DE); Incyte Corporation, Wilmington, DE (US)

(72) Inventors: Günter FINGERLE-ROWSON, Starnberg (DE); Wolfram BRUGGER, Munch (DE); Oliver MANZKE, Morges (CH); Francis SEGUY, Morges (CH)

(21) Appl. No.: 17/541,627

(22) Filed: Dec. 3, 2021

(30) Foreign Application Priority Data
Dec. 4, 2020 (EP) 20211862.6
Feb. 23, 2021 (EP) 21158806.6
Mar. 19, 2021 (EP) 21163696.4
May 7, 2021 (EP) 21172671.6
Jun. 2, 2021 (EP) 21177336.1
Oct. 29, 2021 (EP) 21205447.2

(51) Int. Cl. A61K 39/395; A61K 31/675; A61K 31/704; A61K 31/475; A61K 31/573; A61K 38/19; A61K 31/454; C07K 16/28; A61P 35/00

(52) U.S. Cl. CPC A61K 39/3955; A61K 31/675; A61K 31/704; A61K 31/475; A61K 2039/545; A61K 38/193; A61K 31/454; C07K 16/2803; A61P 35/00; A61K 31/573

(57) ABSTRACT

The present disclosure is directed to a therapeutic combination of an anti-CD19 antibody and R-CHOP or an anti-CD19 antibody, lenalidomide, and R-CHOP for use in the treatment of diffuse large B cell lymphoma. The present disclosure is also directed to a therapeutic combination of an anti-CD19 antibody, lenalidomide, and rituximab for use in the treatment of non-Hodgkin lymphoma, chronic lymphocytic leukemia, or acute lymphoblastic leukemia.

This patent application claims the benefit of priority from EP 20 21 1862.6 filed on Dec. 4, 2020, EP 21 15 8806.6 filed on Feb. 23, 2021, EP 21 16 3696.4 filed on Mar. 19, 2021, EP 21 17 2671.6 filed on May 7, 2021, EP 21 17 7336.1 filed on Jun. 2, 2021 and EP 21 20 5447.2, filed on Oct. 29, 2021, teachings of each of which are incoporated herein by reference in their entirety.

### FIELD

The present disclosure is directed to a therapeutic combination of an anti-CD19 antibody and rituximab, cyclophosphamide, doxorubicin, vincristine, and prednisone/prednisolone (R-CHOP) for use in the treatment of hematological cancer patients. The present disclosure is also directed to a therapeutic combination of an anti-CD19 antibody, lenalidomide, and R-CHOP for use in the treatment of hematological cancer patients. The present disclosure is also directed to a therapeutic combination of an anti-CD19 antibody, lenalidomide, and rituximab for use in the treatment of hematological cancer patients.

### BACKGROUND

Non-Hodgkin lymphoma (NHL) is the most common hematologic malignancy in adults. The majority of NHLs are of B-cell origin, with multiple different histologic subtypes (according to the WHO 2016 classification) that confer different clinical outcomes. Generally, NHL can be divided into aggressive and indolent lymphomas.

Diffuse large B-cell lymphoma (DLBCL) is the most common NHL, representing approximately 40% of all NHLs, and its rate of incidence continues to increase with median age at diagnosis of 64 years. DLBCL is an aggressive B-NHL and the majority of patients present with advanced disease. DLBCL is increasingly recognized as a heterogeneous disorder with distinct cell of origin subtypes, each arising from different stage of normal B-cell development. Several studies have shown that the distinct COO subtypes, Germinal Centre B-cell type (GCB) and activated B-cell type (ABC) have unique mutational profile and also different prognostic outcomes (Lenz, 2008; Flowers, 2010; Vaidya, 2014; Schmitz, 2018).

Indolent NHL comprise approximately one-third of malignant lymphomas. Follicular lymphoma and marginal zone lymphoma are the most common indolent NHL subtypes and account for approximately 20% to 25% and 7% of adult NHL cases, respectively. Both subtypes are considered incurable and have a variable clinical course, with options for management ranging from active surveillance for asymptomatic patients to chemo-immunotherapy, immunotherapy, or treatment with targeted agents for those with symptomatic disease.

The immune-chemotherapy involving administration of the anti-CD20 monoclonal antibody (mAb) rituximab (R) plus CHOP (cyclophosphamide, doxorubicin, vincristine, and prednisone) chemotherapy (R-CHOP) is the current standard of care (SoC) for the treatment of newly diagnosed DLBCL patients. Based on recent data published at ASH2018, there is no added benefit of 8 vs. 6 cycles of R-CHOP in previously untreated DLBCL. Thus, rituximab with 6 cycles of CHOP should be considered SoC (Sehn, 2018). Although the addition of rituximab to CHOP dramatically improved outcomes compared with CHOP alone, still 30-40% DLBCL patients are primary refractory or relapse (Habermann, 2006; Coiffier, 2010). Although some patients with relapsed/refractory DLBCL can be salvaged with second line chemotherapy followed by consolidation with high dose chemotherapy and autologous stem cell transplantation (ASCT), the majority will succumb to the disease. Thus, the development of a more effective initial therapy is needed to improve long-term outcomes.

Various alternative therapies have been explored in an attempt to improve the efficacy of R-CHOP e.g. by increasing the intensity of the chemotherapy, increasing the dose of rituximab, adding maintenance therapy, adding targeted agents to R-CHOP or by using consolidation with high dose therapy and ASCT in the initial management of DLBCL. However, these approaches have largely been unsuccessful, including attempt at maximising dose density with R-CHOP 14 (Pfreundschuh, 2008; Cunningham, 2013; Delarue, 2013). Recent large, randomized, phase III DLBCL trials including B021005/GOYA, comparing the efficacy and safety of Obinutuzumab plus CHOP (G-CHOP) with R-CHOP (Vitolo, 2017), DA-EPOCH-R (Wilson, 2016), and REMARC, comparing lenalidomide maintenance with placebo (Thieblemont, 2016) did not meet its endpoints, reflecting a continued need to improve upon SoC therapies.

CD19 is a 95-kDa transmembrane glycoprotein of the immunoglobulin superfamily containing two extracellular immunoglobulin-like domains and an extensive cytoplasmic tail. The protein is a pan-B lymphocyte surface receptor and is ubiquitously expressed from the earliest stages of pre-B cell development onwards until it is down-regulated during terminal differentiation into plasma cells. It is B-lymphocyte lineage specific and not expressed on hematopoietic stem cells and other immune cells, except some follicular dendritic cells. CD19 functions as a positive regulator of B cell receptor (BCR) signalling and is involved in B cell activation and proliferation and in the development of humoral immune responses. It acts as a co-stimulatory molecule in conjunction with CD21 and CD81 and is involved in B cell responses to T-cell-dependent antigens. The cytoplasmic tail of CD19 is physically associated with a family of tyrosine kinases that trigger downstream signalling pathways via the src-family of protein tyrosine kinases. CD19 is an attractive target for cancers of lymphoid origin since it is highly expressed in nearly all-chronic lymphocytic leukemia (CLL) and non-Hodgkin's lymphomas (NHL), as well as many other different types of leukemias, including acute lymphocytic leukemia (ALL) and hairy cell leukemia (HCL).

Tafasitamab (former names: MOR00208 and XmAb®5574) is a humanized monoclonal antibody that targets the antigen CD19. Tafasitamab has been engineered in the IgG Fc-region to enhance antibody-dependent cell-mediated cytotoxicity (ADCC), thus improving a key mechanism for tumor cell killing and offering potential for enhanced efficacy compared to conventional antibodies, i.e. non-enhanced antibodies. Tafasitamab has or is currently being studied in several clinical trials, such as in CLL, ALL and NHL. In some of those trials, Tafasitamab is used in combination with Idelalisib, Bendamustine, Venetoclax, or lenalidomide.

In the phase 2 L-MIND study (NCT02399085), the efficacy of Tafasitamab in combination with lenalidomide (LEN) was evaluated in adult patients with Relapsed or Refractory Diffuse Large B-cell Lymphoma (rr-DLBCL). L-MIND enrolled 81 patients with DLBCL ineligible for ASCT, who relapsed after or were refractory to 1-3 systemic regimens. Patients received co-administered Tafasitamab (12 mg/kg) and lenalidomide (25 mg/day) for up to 12 cycles (28-days each), followed by MOR00208 monotherapy (in patients with stable disease or better) until disease progression. The primary endpoint was objective response rate (centrally assessed). In this population of patients with relapsed or refractory DLBCL ineligible for stem cell transplant, combination treatment with Tafasitamab and lenalidomide elicited an overall objective response in 60% of patients and a complete response in 42.5%.

The present disclosure concerns treating patients with Diffuse Large B-cell Lymphoma (DLBCL) by administering a combination of an anti-CD19 antibody and R-CHOP. The present disclosure also concerns treating patients with DLBCL by administering a combination of an anti-CD19 antibody, lenalidomide, and R-CHOP. The present disclosure also concerns treating patients with previously untreated DLBCL. The present disclosure also concerns treating patients with non-Hodgkin lymphoma, chronic lymphocytic leukemia, or acute lymphoblastic leukemia by administering a combination of an anti-CD19 antibody, lenalidomide, and rituximab.

### SUMMARY

In certain aspects, the present disclosure provides a treatment for a patient with DLBCL comprising administering to the patient a combination of an anti-CD19 antibody and R-CHOP. In specific embodiments of the combinations as described herein, the combination is synergistic. In specific embodiments of the combinations as described herein, the combination of an anti-CD19 antibody described herein (e.g., tafasitamab) and R-CHOP is synergistic.

In certain aspects, the present disclosure provides a treatment for a patient with DLBCL comprising administering to the patient a combination of an anti-CD19 antibody, lenalidomide, and R-CHOP. In specific embodiments of the combinations as described herein, the combination of an anti-CD19 antibody described herein (e.g., tafasitamab), lenalidomide, and R-CHOP is synergistic. In a specific aspect of the combinations as described herein, the combination of an anti-CD19 antibody described herein (e.g., tafasitamab), lenalidomide, and R-CHOP is synergistic in comparison to the combination of an anti-CD19 antibody and lenalidomide without R-CHOP. In one aspect, the present disclosure provides a treatment for a patient with DLBCL comprising administering to the patient a combination of an anti-CD19 antibody and R-CHOP wherein the anti-CD19 antibody comprises a heavy chain variable region comprising an HCDR1 region comprising the sequence SYVMH (SEQ ID NO: 1), an HCDR2 region comprising the sequence NPYNDG (SEQ ID NO: 2), and an HCDR3 region comprising the sequence GTYYYGTRVFDY (SEQ ID NO: 3) and a light chain variable region comprising the sequence LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

In one aspect, the present disclosure provides a treatment for a patient with DLBCL comprising administering to the patient a combination of an anti-CD19 antibody and R-CHOP wherein the anti-CD19 antibody comprises a heavy chain variable region comprising an HCDR1 region of SYVMH (SEQ ID NO: 1), an HCDR2 region of NPYNDG (SEQ ID NO: 2), and an HCDR3 region of GTYYYGTRVFDY (SEQ ID NO: 3) and a light chain variable region comprising an LCDR1 region of RSSKSLQNVNGNTYLY (SEQ ID NO: 4), an LCDR2 region of RMSNLNS (SEQ ID NO: 5), and an LCDR3 region of MQHLEYPIT (SEQ ID NO: 6).

In one aspect, the present disclosure provides a treatment for a patient with DLBCL comprising administering to the patient a combination of an anti-CD19 antibody, lenalidomide and R-CHOP wherein the anti-CD19 antibody comprises a heavy chain variable region comprising an HCDR1 region comprising the sequence SYVMH (SEQ ID NO: 1), an HCDR2 region comprising the sequence NPYNDG (SEQ ID NO: 2), and an HCDR3 region comprising the sequence GTYYYGTRVFDY (SEQ ID NO: 3) and a light chain variable region comprising the sequence LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

In one aspect, the present disclosure provides a treatment for a patient with DLBCL comprising administering to the patient a combination of an anti-CD19 antibody, lenalidomide and R-CHOP wherein the anti-CD19 antibody comprises a heavy chain variable region comprising an HCDR1 region of SYVMH (SEQ ID NO: 1), an HCDR2 region of NPYNDG (SEQ ID NO: 2), and an HCDR3 region of GTYYYGTRVFDY (SEQ ID NO: 3) and a light chain variable region comprising an LCDR1 region of RSSKSLQNVNGNTYLY (SEQ ID NO: 4), an LCDR2 region of RMSNLNS (SEQ ID NO: 5), and an LCDR3 region of MQHLEYPIT (SEQ ID NO: 6).

In certain embodiments, the anti-CD19 antibody comprises a heavy chain variable region of

(SEQ ID NO: 7)
EVQLVESGGGLVKPGGSLKLSCAASGYTFTSYVMHVWRQAPGKGLEWI
GYINPYNDGTKYNEKFQGRVTISSDKSISTAYMELSSLRSEDTAMYYC
ARGTYYYGTRVFDYWGQGTLVTVSS

and a light chain variable region of

(SEQ ID NO: 8)
DIVMTQSPATLSLSPGERATLSCRSSKSLQNVNGNTYLYWFQQKPGQS
PQLLIYRMSNLNSGVPDRFSGSGSGTEFTLTISSLEPEDFAVYYCMQH
LEYPITFGAGTKLEIK.

In certain embodiments, the anti-CD19 antibody has effector function. In another aspect anti-CD19 antibody has an enhanced effector function. In one embodiment the effector function is ADCC. In one embodiment the anti-CD19 antibody has an enhanced ADCC activity. In a further embodiment the anti-CD19 antibody comprises an Fc domain comprising an amino acid substitution at position S239 and/or 1332, wherein the numbering is according to the EU index as in Kabat.

In certain embodiments, the anti-CD19 antibody comprises a heavy chain constant region of

(SEQ ID NO: 9)
ASTKGPSVFPLAPSSKSTSGGTAALGCLVKDYFPEPVWSWNSGALTSG
VHTFPAVLQSSGLYSLSSVVTVPSSSLGTQTYICNVNHKPSNTKVDKK
VEPKSCDKTHTCPPCPAPELLGGPDVFLFPPKPKDTLMISRTPEVTCW
VDVSHEDPEVQFNVVYVDGVEVHNAKTKPREEQFNSTFRVVSVLTWHQ
DWLNGKEYKCKVSNKALPAPEEKTISKTKGQPREPQVYTLPPSREEMT
KNQVSLTCLVKGFYPSDIAVEWESNGQPENNYKTTPPMLDSDGSFFLY
SKLTVDKSRWQQGNVFSCSVMHEALHNHYTQKSLSLSPGK.

In certain embodiments, the anti-CD19 antibody comprises a light chain constant region of

(SEQ ID NO: 10)
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSS
PVTKSFNRGEC.

In certain embodiments, the anti-CD19 antibody comprises a heavy chain constant region of ASTKGPSVFPLAPSSKSTSGGTAALGCLVKDYFPEPVTVSWNSGALTSGVHTFPAVLQSSGLY SLSSVVTVPSSSLGTQTYICNVNHKPSNTKVDKKVEPKSCDKTHTCPPCPAPELLGGPDVFLFP PKPKDTLMISRTPEVTCVVVDVSHEDPEVQFNWYVDGVEVHNAKTKPREEQFNSTFRVVSVLT VVHQDWLNGKEYKCKVSNKALPAPEEKTISKTKGQPREPQVYTLPPSREEMTKNQVSLTCLVK GFYPSDIAVEWESNGQPENNYKTTPPMLDSDGSFFLYSKLTVDKSRWQQGNVFSCSVMHEAL HNHYTQKSLSLSPGK (SEQ ID NO: 9) and a light chain constant region of

(SEQ ID NO: 10)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

In certain embodiments, the anti-CD19 antibody comprises a heavy chain region of EVQLVESGGGLVKPGGSLKLSCAASGYTFTSYVMHWVRQAPGKGLEWIGYINPYNDGTKYNE KFQGRVTISSDKSISTAYMELSSLRSEDTAMYYCARGTYYYGTRVFDYWGQGTLVTVSSASTK GPSVFPLAPSSKSTSGGTAALGCLVKDYFPEPVTVSWNSGALTSGVHTFPAVLQSSGLYSLSS VVTVPSSSLGTQTYICNVNHKPSNTKVDKKVEPKSCDKTHTCPPCPAPELLGGPDVFLFPPKPK DTLMISRTPEVTCVVVDVSHEDPEVQFNWYVDGVEVHNAKTKPREEQFNSTFRVVSVLTVVHQ DWLNGKEYKCKVSNKALPAPEEKTISKTKGQPREPQVYTLPPSREEMTKNQVSLTCLVKGFYP SDIAVEWESNGQPENNYKTTPPMLDSDGSFFLYSKLTVDKSRWQQGNVFSCSVMHEALHNHY TQKSLSLSPGK (SEQ ID NO: 11) and a light chain region of

(SEQ ID NO: 12)
DIVMTQSPATLSLSPGERATLSCRSSKSLQNVNGNTYLYWFQQKPGQSP
QLLIYRMSNLNSGVPDRFSGSGSGTEFTLTISSLEPEDFAVYYCMQHLE
YPITFGAGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

In certain embodiments, the anti-CD19 antibody consists of a heavy chain region of EVQLVESGGGLVKPGGSLKLSCAASGYTFTSYVMHVVVRQAPGKGLEWIGYINPYNDGTKYNE KFQGRVTISS DKSISTAYMELSSLRSEDTAMYYCARGTYYYGTRVFDYWGQGTLVTVSSASTK GPSVFPLAPSSKSTSGGTAALGCLVKDYFPEPVTVSWNSGALTSGVHTFPAVLQSSGLYSLSS VVTVPSSSLGTQTYICNVNHKPSNTKVDKKVEPKSCDKTHTCPPCPAPELLGGPDVFLFPPKPK DTLMISRTPEVTCVWDVSHEDPEVQFNVVYVDGVEVHNAKTKPREEQFNSTFRWSVLTVVHQ DWLNGKEYKCKVSNKALPAPEEKTISKTKGQPREPQVYTLPPSREEMTKNQVSLTCLVKGFYP SDIAVEWESNGQPENNYKTTPPMLDSDGSFFLYSKLTVDKSRWQQGNVFSCSVMHEALHNHY TQKSLSLSPGK (SEQ ID NO: 11) and a light chain region of

(SEQ ID NO: 12)
DIVMTQSPATLSLSPGERATLSCRSSKSLQNVNGNTYLYWFQQKPGQSP
QLLIYRMSNLNSGVPDRFSGSGSGTEFTLTISSLEPEDFAVYYCMQHLE
YPITFGAGTKLEIKRTVAAPSVFIFPPSDEQLKSGTASWCLLNNFYPRE
AKVQWKVDNALQSGNSQESVTEQDSKDSTYSLSSTLTLSKADYEKHKVY
ACEVTHQGLSSPVTKSFNRGEC.

In certain aspects, the present disclosure provides a method of treating a patient with DLBCL comprising administering to the patient in at least one 21-day cycle a combination of:

tafasitamab in a 12 mg/kg body weight dose on day 1, day 8, and day 15 of the 21-day cycle;

rituximab in a 375 mg/m² dose on day 1 of the 21-day cycle;

cyclophosphamide in a 750 mg/m² dose on day 1 of the 21-day cycle;

doxorubicin in a 50 mg/m² dose on day 1 of the 21-day cycle;

vincristine in a 1.4 to 2.0 mg/m² dose on day 1 of the 21-day cycle; and prednisone or prednisolone in a 100 mg dose on each of days 1 to 5 of the 21-day cycle.

In certain aspects, the present disclosure provides a method of treating a patient with DLBCL comprising administering to the patient a therapeutic amount of a combination of:

tafasitamab;

rituximab;

cyclophosphamide;

doxorubicin;

vincristine;

prednisone or prednisolone; and

granulocyte colony stimulating factor (G-CSF) or pegylated G-CSF.

In specific embodiments of the combinations as described herein, the combination is synergistic.

In one aspect, the invention provides a method of treating a hematological cancer patient with a combination which is a synergistic combination. In one such embodiment, the combination for use in the treatment of a hematological cancer patient comprises: (i) tafasitamab; (ii) rituximab; (iii) cyclophosphamide; (iv) doxorubicin (v) vincristine and (vi) prednisone or prednisolone, wherein tafasitamab (i) and rituximab (ii) are synergistic. In specific embodiments of the combination tafasitamab (i) and cyclophosphamide (iii) are synergistic. In specific embodiments of the combination tafasitamab (i) and doxorubicin (iv) are synergistic. In specific embodiments of the combination tafasitamab (i) and (v) vincristine are synergistic. In specific embodiments of the combination tafasitamab (i) and (vi) prednisone or prednisolone are synergistic. In specific embodiments of the combination (i) tafasitamab; (ii) rituximab; (iii) cyclophosphamide; (iv) doxorubicin (v) vincristine and (vi) prednisone or prednisolone, are synergistic. In an embodiment the synergistic combination further comprises granulocyte colony stimulating factor (G-CSF) or pegylated G-CSF.

In one aspect, the invention provides a method of treating a hematological cancer patient with a combination which is a synergistic combination. In one such embodiment, the synergistic combination for use in the treatment of a hematological cancer patient comprises: (i) tafasitamab; (ii) rituximab; (iii) cyclophosphamide; (iv) doxorubicin (v) vincristine; (vi) prednisone or prednisolone and (vii) lenalidomide, wherein tafasitamab (i) and rituximab (ii) are synergistic. In specific embodiments of the combination tafasitamab (i) and cyclophosphamide (iii) are synergistic. In specific embodiments of the combination tafasitamab (i) and doxorubicin (iv) are synergistic. In specific embodiments of the combination tafasitamab (i) and (v) vincristine are synergistic. In specific embodiments of the combination tafasitamab (i) and (vi) prednisone or prednisolone are synergistic. In specific embodiments of the combination (i) tafasitamab; (ii) rituximab; (iii) cyclophosphamide; (iv) doxorubicin (v) vincristine and (vi) prednisone or prednisolone, are synergistic. In specific embodiments of the combination (i) tafasitamab; (ii) rituximab; (iii) cyclophosphamide; (iv) doxorubicin (v) vincristine, (vi) prednisone or prednisolone and (vii) lenalidomide, are synergistic. In an embodiment the synergistic combination further comprises granulocyte colony stimulating factor (G-CSF) or pegylated G-CSF.

In one aspect, the invention provides a method of treating a hematological cancer patient with a combination which is a synergistic combination. In one such embodiment, the synergistic combination for use in the treatment of a hematological cancer patient comprises: (i) a combination of tafasitamab and lenalidomide and (ii) a combination of rituximab, cyclophosphamide, doxorubicin, vincristine and prednisone or prednisolone (collectively known as R-CHOP), wherein combination (i) and combination (ii) are synergistic. In an embodiment the synergistic combination further comprises granulocyte colony stimulating factor (G-CSF) or pegylated G-CSF.

In one aspect, the invention provides a method of treating a hematological cancer patient with a combination which is a synergistic combination. In one such embodiment, the synergistic combination for use in the treatment of a hematological cancer patient comprises: (i) a combination of tafasitamab and lenalidomide and (ii) a combination of rituximab, cyclophosphamide, doxorubicin, vincristine and prednisone or prednisolone (collectively known as R-CHOP), wherein the combination of combination (i) and combination (ii) has a synergistic effect. In an embodiment the synergistic combination further comprises granulocyte colony stimulating factor (G-CSF) or pegylated G-CSF.

In certain aspects, the present disclosure provides a method of treating a patient with DLBCL comprising administering to the patient in at least one 21-day cycle a combination of:

tafasitamab in a 12 mg/kg body weight dose on day 1, day 8, and day 15 of the 21-day cycle;

lenalidomide in a 25 mg dose on each of days 1 to 10 of the 21-day cycle;

rituximab in a 375 mg/m² dose on day 1 of the 21-day cycle;

cyclophosphamide in a 750 mg/m² dose on day 1 of the 21-day cycle;

doxorubicin in a 50 mg/m² dose on day 1 of the 21-day cycle;

vincristine in a 1.4 to 2.0 mg/m² dose on day 1 of the 21-day cycle; and

prednisone or prednisolone in a 100 mg dose on each of days 1 to 5 of the 21-day cycle.

In certain aspects, the present disclosure provides a method of treating a patient with DLBCL comprising administering to the patient a therapeutic amount of a combination of:

tafasitamab;

lenalidomide;

rituximab;

cyclophosphamide;

doxorubicin;

vincristine;

prednisone or prednisolone; and

granulocyte colony stimulating factor (G-CSF) or pegylated G-CSF.

In certain aspects, the present disclosure provides a method of treating a patient with DLBCL comprising administering to the patient in at least one 21-day cycle a combination of:

tafasitamab in a 12 mg/kg body weight dose on day 1, day 8, and day 15 of the 21-day cycle;

rituximab in a 375 mg/m² dose on day 1 of the 21-day cycle;

cyclophosphamide in a 750 mg/m² dose on day 1 of the 21-day cycle;

doxorubicin in a 50 mg/m² dose on day 1 of the 21-day cycle;

vincristine in a 1.4 to 2.0 mg/m² dose on day 1 of the 21-day cycle; and

prednisone or prednisolone in a 100 mg dose on each of days 1 to 5 of the 21-day cycle; wherein the patient has an International Prognostic Index (IPI) status of 2-5, 3-5, 4-5, 3-4, 3, 4, or 5 prior to starting the administering.

In certain aspects, the present disclosure provides a method of treating a patient with DLBCL comprising administering to the patient a therapeutic amount of a combination of:

tafasitamab;

rituximab;

cyclophosphamide;

doxorubicin;

vincristine;

prednisone or prednisolone; and

granulocyte colony stimulating factor (G-CSF) or pegylated G-CSF; wherein the patient has an International Prognostic Index (IPI) status of 2-5, 3-5, 4-5, 3-4, 3, 4, or 5 prior to starting the administering.

In certain aspects, the present disclosure provides a method of treating a patient with DLBCL comprising administering to the patient in at least one 21-day cycle a combination of:

tafasitamab in a 12 mg/kg body weight dose on day 1, day 8, and day 15 of the 21-day cycle;

lenalidomide in a 25 mg dose on each of days 1 to 10 of the 21-day cycle;

rituximab in a 375 mg/m² dose on day 1 of the 21-day cycle;

cyclophosphamide in a 750 mg/m² dose on day 1 of the 21-day cycle;

doxorubicin in a 50 mg/m² dose on day 1 of the 21-day cycle;

vincristine in a 1.4 to 2.0 mg/m² dose on day 1 of the 21-day cycle; and

prednisone or prednisolone in a 100 mg dose on each of days 1 to 5 of the 21-day cycle;

wherein the patient has an International Prognostic Index (IPI) status of 2-5, 3-5, 4-5, 3-4, 3, 4, or 5 prior to starting the administering.

In certain aspects, the present disclosure provides a method of treating a patient with DLBCL comprising administering to the patient a therapeutic amount of a combination of:

tafasitamab;

lenalidomide;

rituximab;

cyclophosphamide;,

doxorubicin;

vincristine;

prednisone or prednisolone; and

granulocyte colony stimulating factor (G-CSF) or pegylated G-CSF;

wherein the patient has an International Prognostic Index (IPI) status of 2-5, 3-5, 4-5, 3-4, 3, 4, or 5 prior to starting the administering.

In certain aspects, the present disclosure provides a method of treating a patient with DLBCL comprising administering to the patient in at least one 21-day cycle a combination of:

tafasitamab in a 12 mg/kg body weight dose on day 1, day 8, and day 15 of the 21-day cycle;

rituximab in a 375 mg/m² dose on day 1 of the 21-day cycle;

cyclophosphamide in a 750 mg/m² dose on day 1 of the 21-day cycle;

doxorubicin in a 50 mg/m² dose on day 1 of the 21-day cycle;

vincristine in a 1.4 to 2.0 mg/m² dose on day 1 of the 21-day cycle; and

prednisone or prednisolone in a 100 mg dose on each of days 1 to 5 of the 21-day cycle;

wherein the patient has Stage III or Stage IV DLBCL prior to starting the administering.

In certain aspects, the present disclosure provides a method of treating a patient with DLBCL comprising administering to the patient a therapeutic amount of a combination of:

tafasitamab;

rituximab;

cyclophosphamide;

doxorubicin;

vincristine;

prednisone or prednisolone; and

granulocyte colony stimulating factor (G-CSF) or pegylated G-CSF;

wherein the patient has Stage III or Stage IV DLBCL prior to starting the administering.

In certain aspects, the present disclosure provides a method of treating a patient with DLBCL comprising administering to the patient in at least one 21-day cycle a combination of:

tafasitamab in a 12 mg/kg body weight dose on day 1, day 8, and day 15 of the 21-day cycle;

lenalidomide in a 25 mg dose on each of days 1 to 10 of the 21-day cycle;

rituximab in a 375 mg/m² dose on day 1 of the 21-day cycle;

cyclophosphamide in a 750 mg/m² dose on day 1 of the 21-day cycle;

doxorubicin in a 50 mg/m² dose on day 1 of the 21-day cycle;

vincristine in a 1.4 to 2.0 mg/m² dose on day 1 of the 21-day cycle; and

prednisone or prednisolone in a 100 mg dose on each of days 1 to 5 of the 21-day cycle;

wherein the patient has Stage III or Stage IV DLBCL prior to starting the administering.

In certain aspects, the present disclosure provides a method of treating a patient with DLBCL comprising administering to the patient a therapeutic amount of a combination of:

tafasitamab;

lenalidomide;

rituximab;

cyclophosphamide;

doxorubicin;

vincristine;

prednisone or prednisolone; and

granulocyte colony stimulating factor (G-CSF) or pegylated G-CSF;

wherein the patient has Stage III or Stage IV DLBCL prior to starting the administering.

In certain aspects, the present disclosure concerns a therapeutic combination of:

tafasitamab in a 12 mg/kg body weight dose;

rituximab in a 375 mg/m² dose;

cyclophosphamide in a 750 mg/m² dose;

doxorubicin in a 50 mg/m² dose;

vincristine in a 1.4 to 2.0 mg/m² dose; and

prednisone or prednisolone in a 100 mg dose.

In certain aspects, the present disclosure concerns a therapeutic combination of:

tafasitamab in a 12 mg/kg body weight dose;

lenalidomide in a 25 mg dose;

rituximab in a 375 mg/m² dose;

cyclophosphamide in a 750 mg/m² dose;

doxorubicin in a 50 mg/m² dose;

vincristine in a 1.4 to 2.0 mg/m² dose; and

prednisone or prednisolone in a 100 mg dose.

In certain aspects, the present disclosure concerns a therapeutic combination of:

tafasitamab;

rituximab;

cyclophosphamide;

doxorubicin;

vincristine;

prednisone or prednisolone; and

granulocyte colony stimulating factor (G-CSF) or pegylated G-CSF.

In certain aspects, the present disclosure concerns a therapeutic combination of:

tafasitamab;

lenalidomide;

rituximab;

cyclophosphamide;

doxorubicin;

vincristine;

prednisone or prednisolone; and

granulocyte colony stimulating factor (G-CSF) or pegylated G-CSF.

In certain embodiments, tafasitamab is replaced with an anti-CD19 antibody comprising an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO; 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

In certain embodiments, tafasitamab is replaced with an anti-CD19 antibody comprising a variable heavy chain of the sequence

(SEQ ID NO: 7)
EVQLVESGGGLVKPGGSLKLSCAASGYTFTSYVMHVVVRQAPGKGLEW
IGYINPYNDGTKYNEKFQGRVTISSDKSISTAYMELSSLRSEDTAMYY
CARGTYYYGTRVFDYWGQGTLVTVSS

and a variable light chain of the sequence

(SEQ ID NO: 8)
DIVMTQSPATLSLSPGERATLSCRSSKSLQNVNGNTYLYWFQQKPGQS
PQLLIYRMSNLNSGVPDRFSGSGSGTEFTLTISSLEPEDFAVYYCMQH
LEYPITFGAGTKLEIK.

In certain embodiments, the anti-CD19 antibody replacing tafasitamab is a human, humanized, or chimeric antibody. In another embodiment of the present disclosure the anti-CD19 antibody replacing tafasitamab is of the IgG isotype. In another embodiment the antibody replacing tafasitamab is IgG1, IgG2, or IgG1/IgG2 chimeric. In another embodiment of the present disclosure the isotype of the anti-CD19 antibody replacing tafasitamab is engineered to enhance antibody-dependent cell-mediated cytotoxicity. In another embodiment the heavy chain constant region of the anti-CD19 antibody replacing tafasitamab comprises amino acids 239D and 332E, wherein the Fc numbering is according to the EU index as in Kabat. In another embodiment the anti-CD19 antibody replacing tafasitamab is IgG1, IgG2 or IgG1/IgG2, and the chimeric heavy chain constant region of the anti-CD19 antibody comprises amino acids 239D and 332E, wherein the Fc numbering is according to the EU index as in Kabat.

In another aspect, the present disclosure provides a method of treating a non-Hodgkin lymphoma, chronic lymphocytic leukemia, or acute lymphoblastic leukemia in a human subject in need thereof by administering to the human subject a therapeutically effective amount of an antibody that binds to human CD19, lenalidomide, and rituximab. In specific embodiments the administration of a therapeutically effective amount of an antibody that binds to human CD19, lenalidomide, and rituximab has a synergistic effect.

In another aspect, the present disclosure provides a method of treating a non-Hodgkin lymphoma, chronic lymphocytic leukemia, or acute lymphoblastic leukemia in a human subject in need thereof by administering to the human subject a combination which is a synergistic combination. In one such embodiment, the synergistic combination comprises: (i) tafasitamab (ii) lenalidomide and (iii) rituximab, wherein tafasitamab (i) and lenalidomide (ii) are synergistic; wherein (i) tafasitamab and (iii) rituximab are synergistic; wherein (i) tafasitamab (ii) lenalidomide and (iii) rituximab are synergistic. In an embodiment the synergistic combination further comprises granulocyte colony stimulating factor (G-CSF) or pegylated G-CSF.

In one aspect, the invention provides a method of treating a non-Hodgkin lymphoma, chronic lymphocytic leukemia, or acute lymphoblastic leukemia in a human subject in need thereof by administering to the human subject a combination which is a synergistic combination. In one such embodiment, the synergistic combination comprises:
(i) a combination of tafasitamab and lenalidomide and (ii) rituximab, wherein the combination of combination (i) and rituximab (ii) has a synergistic effect. In an embodiment the synergistic combination further comprises granulocyte colony stimulating factor (G-CSF) or pegylated G-CSF.

In some embodiments, the anti-CD19 antibody comprises a variable heavy (VH) domain comprising VH complementarity determining region (CDR)1, VH CDR2, and VH CDR3, wherein:

the VH CDR1 comprises the amino acid sequence SYVMH (SEQ ID NO:1);

the VH CDR2 comprises the amino acid sequence NPYNDG (SEQ ID NO:2); and

the VH CDR3 comprises the amino acid sequence GTYYYGTRVFDY (SEQ ID NO:3); and

wherein the antibody comprises a variable light (VL) domain comprising VL CDR1, VL CDR2, and VL CDR3, wherein:

the VL CDR1 comprises the amino acid sequence RSSKSLQNVNGNTYLY (SEQ ID NO:4);

the VL CDR2 comprises the amino acid sequence RMSNLNS (SEQ ID NO:5); and

the VL CDR3 comprises the amino acid sequence MQHLEYPIT (SEQ ID NO:6).

In some embodiments, the VH domain comprises the amino acid sequence EVQLVESGGGLVKPGGSLKLSCAASGYTFTSYVMHWVRQAPGKGLEWIGYINPYNDGTKYNE KFQGRVTISSDKSISTAYMELSSLRSEDTAMYYCARGTYYYGTRVFDYWGQGTLVTVSS (SEQ ID NO:7) and the VL domain comprises the amino acid sequence

(SEQ ID NO: 8)
DIVMTQSPATLSLSPGERATLSCRSSKSLQNVNGNTYLYWFQQKPGQS
PQLLIYRMSNLNSGVPDRFSGSGSGTEFTLTISSLEPEDFAVYYCMQH
LEYPITFGAGTKLEIK.

In some embodiments, the anti-CD19 antibody comprises a heavy chain and a light chain, and wherein the heavy chain comprises the amino acid sequence set forth in SEQ ID NO:11 and the light chain comprises the amino acid sequence set forth in SEQ ID NO:12.

In some embodiments, the human subject has a non-Hodgkin lymphoma (e.g., relapsed/refractory non-Hodgkin lymphoma).

In some embodiments, the non-Hodgkin lymphoma is follicular lymphoma (e.g., relapsed/refractory follicular lymphoma, histologically confirmed Grade 1, 2, or 3a follicular lymphoma, or histologically confirmed Grade 1, 2, or 3a relapsed/refractory follicular lymphoma).

In some embodiments, the anti-CD19 antibody is tafasitamab and the non-Hodgkin lymphoma is follicular lymphoma (e.g., histologically confirmed Grade 1, 2, or 3a follicular lymphoma).

In some embodiments, the anti-CD19 antibody is tafasitamab and the non-Hodgkin lymphoma is relapsed/refractory follicular lymphoma (e.g., histologically confirmed Grade 1, 2, or 3a relapsed/refractory follicular lymphoma).

In some embodiments, the non-Hodgkin lymphoma is marginal zone lymphoma (e.g., relapsed/refractory marginal zone lymphoma, histologically confirmed nodal marginal zone lymphoma, splenic marginal zone lymphoma, extranodal marginal zone lymphoma of the mucosa-associated lymphoid tissue, histologically confirmed nodal relapsed/refractory marginal zone lymphoma, splenic relapsed/refractory marginal zone lymphoma, or extranodal relapsed/refractory marginal zone lymphoma of the mucosa-associated lymphoid tissue).

In some embodiments, the anti-CD19 antibody is tafasitamab and the non-Hodgkin lymphoma is marginal zone lymphoma (e.g., histologically confirmed nodal marginal zone lymphoma, splenic marginal zone lymphoma, extranodal marginal zone lymphoma of the mucosa-associated lymphoid tissue).

In some embodiments, the anti-CD19 antibody is tafasitamab and the non-Hodgkin lymphoma is relapsed/refractory marginal zone lymphoma (e.g., histologically confirmed nodal relapsed/refractory marginal zone lymphoma, splenic relapsed/refractory marginal zone lymphoma, or extranodal relapsed/refractory marginal zone lymphoma of the mucosa-associated lymphoid tissue).

In some embodiments, the non-Hodgkin lymphoma is diffuse large B-cell lymphoma (e.g., relapsed/refractory diffuse large B-cell lymphoma).

In some embodiments, the anti-CD19 antibody is tafasitamab and the non-Hodgkin lymphoma is diffuse large B-cell lymphoma (e.g., relapsed/refractory diffuse large B-cell lymphoma).

In some embodiments, the non-Hodgkin lymphoma is small lymphocytic lymphoma (e.g., relapsed/refractory small lymphocytic lymphoma).

In some embodiments, the anti-CD19 antibody is tafasitamab and the non-Hodgkin lymphoma is small lymphocytic lymphoma (e.g., relapsed/refractory small lymphocytic lymphoma).

In some embodiments, the non-Hodgkin lymphoma is mucosa-associated lymphoid tissue lymphoma (e.g., relapsed/refractory mucosa-associated lymphoid tissue lymphoma).

In some embodiments, the anti-CD19 antibody is tafasitamab and the non-Hodgkin lymphoma is mucosa-associated lymphoid tissue lymphoma (e.g., relapsed/refractory mucosa-associated lymphoid tissue lymphoma).

In some embodiments, the non-Hodgkin lymphoma is Burkitt's lymphoma (e.g., relapsed/refractory Burkitt's lymphoma).

In some embodiments, the anti-CD19 antibody is tafasitamab and the non-Hodgkin lymphoma is Burkitt's lymphoma (e.g., relapsed/refractory Burkitt's lymphoma).

In some embodiments, the non-Hodgkin lymphoma is mantle cell lymphoma (e.g., relapsed/refractory mantle cell lymphoma).

In some embodiments, the anti-CD19 antibody is tafasitamab and the non-Hodgkin lymphoma is mantle cell lymphoma (e.g., relapsed/refractory mantle cell lymphoma).

In some embodiments, the human subject has chronic lymphocytic leukemia (e.g., relapsed/refractory chronic lymphocytic leukemia).

In some embodiments, the anti-CD19 antibody is tafasitamab and the human subject has chronic lymphocytic leukemia (e.g., relapsed/refractory chronic lymphocytic leukemia).

In some embodiments, the human subject has acute lymphoblastic leukemia (e.g., relapsed/refractory acute lymphoblastic leukemia).

In some embodiments, the anti-CD19 antibody is tafasitamab and the human subject has acute lymphoblastic leukemia (e.g., relapsed/refractory acute lymphoblastic leukemia).

In some embodiments, the anti-CD19 antibody (e.g., tafasitamab) is administered intravenously. In some embodiments, the anti-CD19 antibody (e.g., tafasitamab) is administered intravenously at a dose 12 mg/kg. In some embodiments, the anti-CD19 antibody (e.g., tafasitamab) is administered intravenously at least once every two weeks at a dose of 12 mg/kg. In some embodiments, the anti-CD19 antibody (e.g., tafasitamab) is administered intravenously at a dose of 12 mg/kg according to the following schedule:

on days 1, 8, 15, and 22 of a first 28-day cycle;

on days 1, 8, 15, and 22 of a second 28-day cycle;

on days 1, 8, 15, and 22 of a third 28-day cycle; and

on days 1 and 15 of a fourth 28-day cycle and on days 1 and 15 of further 28-day cycles thereafter.

In some embodiments, rituximab is administered intravenously. In some embodiments, rituximab is administered intravenously at a dose of 375 mg/m². In some embodiments, rituximab is administered intravenously at a dose of 375 mg/m² according to the following schedule:

on days 1, 8, 15, and 22 of a first 28-day cycle; and

on day 1 of a second 28-day cycle and on day 1 of further 28-day cycles thereafter.

In some embodiments, lenalidomide is administered orally. In some embodiments, lenalidomide is administered orally at a dose of 20 mg. In some embodiments, lenalidomide is administered orally at a dose of 20 mg on days 1-21 of repeated 28-day cycles.

In some embodiments, the anti-CD19 antibody (e.g., tafasitamab) is administered intravenously, rituximab is administered intravenously, and lenalidomide is administered orally.

In some embodiments, the anti-CD19 antibody (e.g., tafasitamab) is administered intravenously at a dose 12 mg/kg, rituximab is administered intravenously at a dose of 375 mg/m², and lenalidomide is administered orally at a dose of 20 mg.

In some embodiments, the anti-CD19 antibody (e.g., tafasitamab) is administered intravenously at a dose 12 mg/kg according to the following schedule:

on days 1, 8, 15, and 22 of a first 28-day cycle;

on days 1, 8, 15, and 22 of a second 28-day cycle;

on days 1, 8, 15, and 22 of a third 28-day cycle; and

on days 1 and 15 of a fourth 28-day cycle and on days 1 and 15 of further 28-day cycles thereafter,

rituximab is administered intravenously at a dose of 375 mg/m² according to the following schedule:

on days 1, 8, 15, and 22 of a first 28-day cycle; and

on day 1 of a second 28-day cycle and on day 1 of further 28-day cycles thereafter, and

lenalidomide is administered orally at a dose of 20 mg on days 1-2.1 of repeated 28-day cycles.

### BRIEF DESCRIPTION OF THE DRAWINGS

FIG. 1 is a schematic of the trial design discussed in Example 1.

FIG. 2 provides a summary of TEAEs by System Organ Class (SOC).

FIG. 3 provides a summary of neutrophil and platelet counts.

FIG. 4 provides the overall Study Design of the Front-MIND trial.

FIG. 5 provides the study scheme and treatment schedule

### DETAILED DESCRIPTION

The term “CD19” refers to the protein known as CD19, having the following synonyms: B4, B-lymphocyte antigen CD19, B-lymphocyte surface antigen B4, CVID3, Differentiation antigen CD19, MGC12802, and T-cell surface antigen Leu-12.

Human CD19 has the amino acid sequence of:

(SEQ ID NO: 13)
MPPPRLLFFLLFLTPMEVRPEEPLVVKVEEGDNAVLQCLKGTSDGPTQQ
LTVVSRESPLKPFLKLSLGLPGLGIHMRPLAIWLFIFNVSQQMGGFYLC
QPGPPSEKAWQPGWTVNVEGSGELFRWNVSDLGGLGCGLKNRSSEGPSS
PSGKLMSPKLYVWAKDRPEIWEGEPPCLPPRDSLNQSLSQDLTMAPGST
LWLSCGVPPDSVSRGPLSVVTHVHPKGPKSLLSLELKDDRPARDMWVME
TGLLLPRATAQDAGKYYCHRGNLTMSFHLEITARPVLWHWLLRTGGWKV
SAVTLAYLIFCLCSLVGILHLQRALVLRRKRKRMTDPTRRFFKVTPPPG
SGPQNQYGNVLSLPTPTSGLGRAQRWAAGLGGTAPSYGNPSSDVQADGA
LGSRSPPGVGPEEEEGEGYEEPDSEEDSEFYENDSNLGQDQLSQDGSGY
ENPEDEPLGPEDEDSFSNAESYENEDEELTQPVARTMDFLSPHGSAWDP
SREATSLGSQSYEDMRGILYAAPQLRSIRGQPGPNHEEDADSYENMDNP
DGPDPAWGGGGRMGTWSTR

Tafasitamab”, “MOR00208” and “XmAb5574” are used as synonyms to describe the antibody of Table 1. Table 1 provides the amino acid sequences of tafasitamab. The full length heavy chain amino acid sequence of tafasitamab is shown as SEQ ID NO: 11 and the full length light chain amino acid sequence of tafasitamab is shown as SEQ ID NO: 12. Tafasitamab is described in U.S. Pat. No. 8,524,867, which is incorporated by reference in its entirety (in U.S. Pat. No. 8,524,867, the full heavy chain of tafasitamab is SEQ ID NO:87 and the full light chain of tafasitamab is SEQ ID NO:106). Tafasitumab includes the U.S. Food and Drug Administration (FDA) approved Monjuvi® (tafasitumab-cxix).

The term “antibody” as used herein refers to a protein comprising at least two heavy (H) chains and two light (L) chains inter-connected by disulfide bonds, which interacts with an antigen. Each heavy chain is comprised of a variable heavy chain region (abbreviated herein as VH) and a heavy chain constant region. The heavy chain constant region is comprised of three domains, CH1, CH2 and CH3. Each light chain is comprised of a variable light chain region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2), subclass or certain combinations thereof. Both the light and heavy chains are divided into regions of structural and functional homology.

The phrase “antibody fragment”, as used herein, refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CHI domains; a F(ab)2 fragment, a bivalent fragment comprising two Fab fragments linked by a disulfide bridge at the hinge region; a Fd fragment consisting of the VH and CHI domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CHI-VH-CHI) which, together with complementary light chain polypeptides, form a pair of antigen-binding sites (Zapata et al., (1995) Protein Eng. 8:1057-1062; and U.S. Pat. No. 5,641,870).

“Administered” or “administration” includes but is not limited to delivery of a drug by an injectable form, such as, for example, an intravenous, intramuscular, intradermal; or subcutaneous route or mucosal route, for example, as a nasal spray or aerosol for inhalation; or as an ingestible solution, for example, as a capsule or tablet.

The term “effector function” refers to those biological activities attributable to the Fc region of an antibody, which vary with the antibody isotype. Non-limiting examples of antibody effector functions include C1q binding and complement dependent cytotoxicity (CDC); Fc receptor binding and antibody-dependent cell-mediated cytotoxicity (ADCC) and/or antibody-dependent cellular phagocytosis (ADCP); down regulation of cell surface receptors (e.g. B cell receptor); and B cell activation.

“Antibody-dependent cell-mediated cytotoxicity” or “ADCC” refers to a form of cytotoxicity in which antibodies bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express Fc□RIII only, whereas monocytes express FcγRI, FcγRII, and FcγRIII.

Non-Hodgkin's lymphoma (“NHL”) is a heterogeneous malignancy originating from lymphocytes. In the United States (U.S.), the incidence is estimated at 65,000/year with mortality of approximately 20,000 (American Cancer Society, 2006; and SEER Cancer Statistics Review). The disease can occur in all ages, the usual onset begins in adults over 40 years, with the incidence increasing with age. NHL is characterized by a clonal proliferation of lymphocytes that accumulate in the lymph nodes, blood, bone marrow and spleen, although any major organ may be involved. The current classification system used by pathologists and clinicians is the World Health Organization (WHO) Classification of Tumors, which organizes NHL into precursor and mature B-cell or T-cell neoplasms. The PDQ is currently dividing NHL as indolent or aggressive for entry into clinical trials. The indolent NHL group is comprised primarily of follicular subtypes, small lymphocytic lymphoma, MALT (mucosa-associated lymphoid tissue), and marginal zone; indolent encompasses approximately 50% of newly diagnosed B-cell NHL patients. Aggressive NHL includes patients with histologic diagnoses of primarily diffuse large B cell (DLBL, “DLBCL”, or DLCL) (40% of all newly diagnosed patients have diffuse large cell), Burkitt's, and mantle cell (“MCL”). The clinical course of NHL is highly variable. A major determinant of clinical course is the histologic subtype. Studies to date have not demonstrated an improvement in survival with early intervention. In asymptomatic patients, it is acceptable to “watch and wait” until the patient becomes symptomatic or the disease pace appears to be accelerating. Over time, the disease may transform to a more aggressive histology. The median survival is 8 to 10 years, and indolent patients often receive 3 or more treatments during the treatment phase of their disease. Initial treatment of the symptomatic indolent NHL patient historically has been combination chemotherapy. The most commonly used agents include: cyclophosphamide, vincristine and prednisone (CVP); or cyclophosphamide, adriamycin, vincristine, prednisone (CHOP). Approximately 70% to 80% of patients will respond to their initial chemotherapy, duration of remissions last on the order of 2-3 years. Ultimately the majority of patients relapse. The discovery and clinical use of the anti-CD20 antibody, rituximab, has provided significant improvements in response and survival rate. The current standard of care for most patients is rituximab +CHOP (R-CHOP) or rituximab +CVP (R-CVP). Rituximab therapy has been shown to be efficacious in several types of NHL, and is currently approved as a first line treatment for both indolent (follicular lymphoma) and aggressive NHL (diffuse large B cell lymphoma). However, there are significant limitations of anti-CD20 monoclonal antibody (mAb), including primary resistance (50% response in relapsed indolent patients), acquired resistance (50% response rate upon re-treatment), rare complete response (2% complete response rate in relapsed population), and a continued pattern of relapse. Finally, many B cells do not express CD20, and thus many B-cell disorders are not treatable using anti-CD20 antibody therapy.

“Subject” or “patient” as used in this context refers to a human patient.

The “Fc region” is used to define the C-terminal region of an immunoglobulin heavy chain. The Fc region of an immunoglobulin generally comprises two constant domains, a CH2 domain and a CH3 domain. Unless otherwise specified herein, numbering of amino acid residues in the Fc region is according to the EU numbering system, also called the EU index, as described in Kabat et al., Sequences of Proteins of Immunological Interest, 5^{th} Ed. Public Health Service, National Institutes of Health, Bethesda, Md., 1991.

The agents which are administered according to the present disclosure are administered to the patient in a therapeutically effective amount. A “therapeutically effective amount” refers to an amount sufficient to provide some improvement of the clinical manifestations of a given disease or disorder.

“Survival” refers to the patient remaining alive, and includes overall survival as well as progression free survival.

“Overall survival” or “OS” refers to the patient remaining alive for a defined period of time, such as 12 months, 24 months, 3 years, 5 years, etc. from the time of diagnosis or treatment.

“Progression free survival” or “PFS” refers to the patient remaining alive, without the cancer progressing or getting worse. Disease progression can be documented by any clinically accepted methods.

By “extending survival” or “improving surviving” is meant increasing overall survival or progression free survival in a patient treated in accordance with the present disclosure relative to an untreated patient and/or relative to a patient treated with one or more approved anti-tumor agents, but not receiving treatment in accordance with the present disclosure.

An “objective response” or “overall response” refers to a measurable response, including complete response (CR) or partial response (PR).

By “complete response” or “CR” is intended the disappearance of all signs of cancer in response to treatment. This does not always mean the cancer has been cured.

“Partial response” or “PR” refers to a decrease in the size of one or more tumors or lesions, or in the extent of cancer in the body, in response to treatment.

“In combination” refers to the administration of one therapy in addition to another therapy. As such, “in combination with” includes simultaneous (e.g., concurrent) and consecutive administration in any order. By way of non-limiting example, a first therapy (e.g., agent, such as an anti-CD19 antibody, like tafasitamab) may be administered before (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., pharmaceutical agent or agents) to a patient. In some embodiments, the term “combination” means that the anti-CD19 antibody and the pharmaceutical agent or agents are administered simultaneously or consecutivley. In certain embodiments, the anti-CD19 antibody and the pharmaceutical agent or agents are administered in separate compositions, i.e., wherein the anti-CD19 antibody and the pharmaceutical agent or agents are administered each in a separate unit dosage form. It is understood that the anti-CD19 antibody and the pharmaceutical agent or agents are administered on the same day or on different days and in any order as according to an appropriate dosing protocol.

“Lenalidomide” has the following structure:

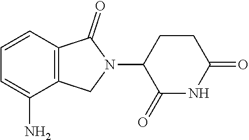 [SMILES: Nc1cccc2c1CN(C1CCC(=O)NC1=O)C2=O]

In one aspect, the present disclosure concerns a method of treating DLBCL in a human subject, comprising administering to the subject a combination of tafasitamab and R-CHOP or a combination of tafasitamab, lenalidomide, and R-CHOP.

The use of a CD19 antibody in non-specific B cell lymphomas is discussed in WO2007076950 (US2007154473), which are both incorporated by reference. The use of a CD19 antibody in CLL, NHL and ALL is described in Scheuermann et al., CD19 Antigen in Leukemia and Lymphoma Diagnosis and Immunotherapy, Leukemia and Lymphoma, Vol. 18, 385-397 (1995), which is incorporated by reference in its entirety.

Additional antibodies specific for CD19 are described in WO2005012493 (U.S. Pat. No. 7,109,304), WO2010053716 (U.S. Ser. No. 12/266,999) (Immunomedics); WO2007002223 (U.S. Pat. No. 8,097,703) (Medarex); WO2008022152 (Ser. No. 12/377,251) and WO2008150494 (Xencor), WO2008031056 (U.S. Ser. No. 11/852,106) (Medimmune); WO 2007076950 (U.S. Ser. No. 11/648,505) (Merck Patent GmbH); WO 2009/052431 (U.S. Ser. No. 12/253,895) (Seattle Genetics); and WO2010095031 (Ser. No. 12/710,442) (Glenmark Pharmaceuticals), WO2012010562 and WO2012010561 (International Drug Development), WO2011147834 (Roche Glycart), and WO 2012/156455 (Sanofi), which are all incorporated by reference in their entireties.

A pharmaceutical composition includes an active agent, e.g. an antibody for therapeutic use in humans. A pharmaceutical composition may additionally include pharmaceutically acceptable carriers or excipients.

### Further Embodiments

The present disclosure provides a pharmaceutical combination comprising an anti-CD19 antibody and R-CHOP for use in the treatment of patients with DLBCL.

The present disclosure further provides a pharmaceutical combination comprising an anti-CD19 antibody, lenalidomide and R-CHOP for use in the treatment of patients with DLBCL.

The present disclosure provides an anti-CD19 antibody for use in the treatment of patients with DLBCL wherein the anti-CD19 antibody is administered in combination with R-CHOP.

The present disclosure provides a pharmaceutical combination comprising tafasitamab and R-CHOP for use in the treatment of patients with DLBCL.

The present disclosure further provides a pharmaceutical combination comprising tafasitamab, lenalidomide, and R-CHOP for use in the treatment of patients with DLBCL.

The present disclosure provides tafasitamab for use in the treatment of patients with DLBCL wherein tafasitamab is administered in combination with R-CHOP.

The present disclosure provides tafasitamab for use in the treatment of patients with DLBCL wherein tafasitamab is administered in combination with lenalidomide and R-CHOP.

The present disclosure provides tafasitamab for use in the treatment of patients with DLBCL wherein tafasitamab is administered in combination with R-CHOP. In specific embodiments the combination of tafasitamab with R-CHOP is synergistic.

The present disclosure provides tafasitamab for use in the treatment of patients with DLBCL wherein tafasitamab is administered in combination with lenalidomide and R-CHOP. In specific embodiments the combination of tafasitamab with lenalidomide and R-CHOP is synergistic. In another specific embodiments the combination of tafasitamab and lenalidomide with R-CHOP has a synergistic effect.

In an embodiment the synergistic combination further comprises granulocyte colony stimulating factor (G-CSF) or pegylated G-CSF.

The present disclosure provides tafasitamab for use in the treatment of patients with DLBCL wherein tafasitamab is administered in combination with

rituximab;

cyclophosphamide;

doxorubicin;

vincristine; and

prednisone or prednisolone is administered in a 100 mg dose.

In certain embodiments lenalidomide is co-administered. In certain embodiments, granulocyte colony stimulating factor (G-CSF) or pegylated G-CSF is co-administered.

The present disclosure provides tafasitamab for use in the treatment of patients with DLBCL comprising administering to the patient in at least one 21-day cycle a combination of:

tafasitamab on day 1, day 8, and day 15 of the 21-day cycle;

rituximab on day 1 of the 21-day cycle;

cyclophosphamide on day 1 of the 21-day cycle;

doxorubicin on day 1 of the 21-day cycle;

vincristine on day 1 of the 21-day cycle; and

prednisone or prednisolone on each of days 1 to 5 of the 21-day cycle.

In certain embodiments, the treatment comprises administering to the patient at least three 21-day cycles of the combination. In certain embodiments, the treatment comprises administering to the patient at least six 21-day cycles of the combination.

The present disclosure provides tafasitamab for use in the treatment of patients with DLBCL wherein tafasitamab is administered in combination with R-CHOP and wherein

tafasitamab is administered in a body weight dose of 8 mg/kg to 40 mg/kg;

rituximab is administered in a 375 mg/m² dose;

cyclophosphamide is administered in a 750 mg/m² dose;

doxorubicin is administered in a 50 mg/m² dose;

vincristine is administered in a 1.4 to 2.0 mg/m² dose; and

prednisone or prednisolone is administered in a 100 mg dose.

In certain embodiments, lenalidomide is co-administered. In certain embodiments, lenalidomide in a 25 mg dose is co-administered. In certain embodiments, granulocyte colony stimulating factor (G-CSF) or pegylated G-CSF is co-administered.

The present disclosure provides tafasitamab for use in the treatment of patients with DLBCL wherein tafasitamab is administered in combination with R-CHOP and wherein

tafasitamab is administered in a 12 mg/kg body weight dose;

rituximab is administered in a 375 mg/m² dose;

cyclophosphamide is administered in a 750 mg/m² dose;

doxorubicin is administered in a 50 mg/m² dose;

vincristine is administered in a 1.4 to 2.0 mg/m² dose; and

prednisone or prednisolone is administered in a 100 mg dose.

In certain embodiments, lenalidomide is co-administered. In certain embodiments, lenalidomide in a 25 mg dose is co-administered. In certain embodiments, granulocyte colony stimulating factor (G-CSF) or pegylated G-CSF is co-administered.

The present disclosure provides tafasitamab for use in the treatment of patients with DLBCL wherein tafasitamab is administered in combination with R-CHOP in at least one 21-day cycle, wherein

tafasitamab is administered in a 12 mg/kg body weight dose on day 1, day 8, and day 15 of the 21-day cycle;

rituximab is administered in a 375 mg/m² dose on day 1 of the 21-day cycle;

cyclophosphamide is administered in a 750 mg/m² dose on day 1 of the 21-day cycle;

doxorubicin is administered in a 50 mg/m² dose on day 1 of the 21-day cycle;

vincristine is administered in a 1.4 to 2.0 mg/m² dose on day 1 of the 21-day cycle; and

prednisone or prednisolone is administered in a 100 mg dose on each of days 1 to 5 of the 21-day cycle.

In certain embodiments, the treatment comprises administering to the patient at least three 21-day cycles of the combination. In certain embodiments, the treatment comprises administering to the patient at least six 21-day cycles of the combination.

In certain embodiments, lenalidomide is co-administered. In certain embodiments, lenalidomide in a 25 mg dose is co-administered on each of days 1 to 10 of the 21-day cycle. In certain embodiments, granulocyte colony stimulating factor (G-CSF) or pegylated G-CSF is co-administered.

In certain aspects, the present disclosure provides a treatment for a patient with DLBCL comprising administering to the patient a combination of an anti-CD19 antibody and R-CHOP.

In certain aspects, the present disclosure provides a treatment for a patient with DLBCL comprising administering to the patient a combination of an anti-CD19 antibody, lenalidomide, and R-CHOP.

In certain aspects, the present disclosure provides a method of treating a patient with DLBCL comprising administering to the patient in at least one 21-day cycle a combination of: tafasitamab in a 12 mg/kg body weight dose on day 1, day 8, and day 15 of the 21-day cycle;

rituximab in a 375 mg/m² dose on day 1 of the 21-day cycle;

cyclophosphamide in a 750 mg/m² dose on day 1 of the 21-day cycle;

doxorubicin in a 50 mg/m² dose on day 1 of the 21-day cycle;

vincristine in a 1.4 to 2.0 mg/m² dose on day 1 of the 21-day cycle; and

prednisone or prednisolone in a 100 mg dose on each of days 1 to 5 of the 21-day cycle.

In certain embodiments, the treatment comprises administering to the patient at least three 21-day cycles of the combination. In certain embodiments, the treatment comprises administering to the patient at least six 21-day cycles of the combination.

In certain aspects, the present disclosure provides a method of treating a patient with DLBCL comprising administering to the patient a therapeutic amount of a combination of:

tafasitamab;

rituximab;

cyclophosphamide;

doxorubicin;

vincristine;

prednisone or prednisolone; and

granulocyte colony stimulating factor (G-CSF) or pegylated G-CSF.

In certain aspects, the present disclosure provides a method of treating a patient with DLBCL comprising administering to the patient in at least one 21-day cycle a combination of:

tafasitamab in a 12 mg/kg body weight dose on day 1, day 8, and day 15 of the 21-day cycle;

lenalidomide in a 25 mg dose on each of days 1 to 10 of the 21-day cycle;

rituximab in a 375 mg/m² dose on day 1 of the 21-day cycle;

cyclophosphamide in a 750 mg/m² dose on day 1 of the 21-day cycle;

doxorubicin in a 50 mg/m² dose on day 1 of the 21-day cycle;

vincristine in a 1.4 to 2.0 mg/m² dose on day 1 of the 21-day cycle; and

prednisone or prednisolone in a 100 mg dose on each of days 1 to 5 of the 21-day cycle.

In certain embodiments, the treatment comprises administering to the patient at least three 21-day cycles of the combination. In certain embodiments, the treatment comprises administering to the patient at least six 21-day cycles of the combination.

In certain aspects, the present disclosure provides a method of treating a patient with DLBCL comprising administering to the patient a therapeutic amount of a combination of:

tafasitamab;

lenalidomide;

rituximab;

cyclophosphamide;

doxorubicin;

vincristine;

prednisone or prednisolone; and granulocyte colony stimulating factor (G-CSF) or pegylated G-CSF.

In certain aspects, the present disclosure provides a method of treating a patient with DLBCL comprising administering to the patient in at least one 21-day cycle a combination of:

tafasitamab in a 12 mg/kg body weight dose on day 1, day 8, and day 15 of the 21-day cycle;

rituximab in a 375 mg/m² dose on day 1 of the 21-day cycle;

cyclophosphamide in a 750 mg/m² dose on day 1 of the 21-day cycle;

doxorubicin in a 50 mg/m² dose on day 1 of the 21-day cycle;

vincristine in a 1.4 to 2.0 mg/m² dose on day 1 of the 21-day cycle; and

prednisone or prednisolone in a 100 mg dose on each of days 1 to 5 of the 21-day cycle;

wherein the patient has an International Prognostic Index (IPI) status of 2-5, 3-5, 4-5, 3-4, 3, 4, or 5 prior to starting the administering.

In certain embodiments, the treatment comprises administering to the patient at least three 21-day cycles of the combination. In certain embodiments, the treatment comprises administering to the patient at least six 21-day cycles of the combination.

In certain aspects, the present disclosure provides a method of treating a patient with DLBCL comprising administering to the patient a therapeutic amount of a combination of:

tafasitamab;

rituximab;

cyclophosphamide;

doxorubicin;

vincristine; prednisone or prednisolone: and

granulocyte colony stimulating factor (G-CSF) or pegylated G-CSF;

wherein the patient has an International Prognostic Index (IPI) status of 2-5, 3-5, 4-5, 3-4, 3, 4, or 5 prior to starting the administering.

In certain aspects, the present disclosure provides a method of treating a patient with DLBCL comprising administering to the patient in at least one 21-day cycle a combination of:

tafasitamab in a 12 mg/kg body weight dose on day 1, day 8, and day 15 of the 21-day cycle;

lenalidomide in a 25 mg dose on each of days 1 to 10 of the 21-day cycle;

rituximab in a 375 mg/m² dose on day 1 of the 21-day cycle;

cyclophosphamide in a 750 mg/m² dose on day 1 of the 21-day cycle;

doxorubicin in a 50 mg/m² dose on day 1 of the 21-day cycle;

vincristine in a 1.4 to 2.0 mg/m² dose on day 1 of the 21-day cycle; and

prednisone or prednisolone in a 100 mg dose on each of days 1 to 5 of the 21-day cycle;

wherein the patient has an International Prognostic Index (IPI) status of 2-5, 3-5, 4-5, 3-4, 3, 4, or 5 prior to starting the administering.

In certain embodiments, the treatment comprises administering to the patient at least three 21-day cycles of the combination. In certain embodiments, the treatment comprises administering to the patient at least six 21-day cycles of the combination.

In certain aspects, the present disclosure provides a method of treating a patient with DLBCL comprising administering to the patient a therapeutic amount of a combination of:

tafasitamab;

lenalidomide;

rituximab;

cyclophosphamide;

doxorubicin;

vincristine;

prednisone or prednisolone; and

granulocyte colony stimulating factor (G-CSF) or pegylated G-CSF;

wherein the patient has an International Prognostic Index (IPI) status of 2-5, 3-5, 4-5, 3-4, 3, 4, or 5 prior to starting the administering.

In certain aspects, the present disclosure provides a method of treating a patient with DLBCL comprising administering to the patient in at least one 21-day cycle a combination of:

tafasitamab in a 12 mg/kg body weight dose on day 1, day 8, and day 15 of the 21-day cycle;

rituximab in a 375 mg/m² dose on day 1 of the 21-day cycle;

cyclophosphamide in a 750 mg/m² dose on day 1 of the 21-day cycle;

doxorubicin in a 50 mg/m² dose on day 1 of the 21-day cycle;

vincristine in a 1.4 to 2.0 mg/m² dose on day 1 of the 21-day cycle; and

prednisone or prednisolone in a 100 mg dose on each of days 1 to 5 of the 21-day cycle;

wherein the patient has Stage III or Stage IV DLBCL prior to starting the administering.

In certain embodiments, the treatment comprises administering to the patient at least three 21-day cycles of the combination. In certain embodiments, the treatment comprises administering to the patient at least six 21-day cycles of the combination.

In certain aspects, the present disclosure provides a method of treating a patient with DLBCL comprising administering to the patient a therapeutic amount of a combination of:

tafasitamab;

rituximab;

cyclophosphamide;

doxorubicin;

vincristine;

prednisone or prednisolone: and

granulocyte colony stimulating factor (G-CSF) or pegylated G-CSF;

wherein the patient has Stage III or Stage IV DLBCL prior to starting the administering.

In certain aspects, the present disclosure provides a method of treating a patient with DLBCL comprising administering to the patient in at least one 21-day cycle a combination of:

tafasitamab in a 12 mg/kg body weight dose on day 1, day 8, and day 15 of the 21-day cycle;

lenalidomide in a 25 mg dose on each of days 1 to 10 of the 21-day cycle;

rituximab in a 375 mg/m² dose on day 1 of the 21-day cycle;

cyclophosphamide in a 750 mg/m² dose on day 1 of the 21-day cycle;

doxorubicin in a 50 mg/m² dose on day 1 of the 21-day cycle;

vincristine in a 1.4 to 2.0 mg/m² dose on day 1 of the 21-day cycle; and

prednisone or prednisolone in a 100 mg dose on each of days 1 to 5 of the 21-day cycle;

wherein the patient has Stage III or Stage IV DLBCL prior to starting the administering.

In certain embodiments, the treatment comprises administering to the patient at least three 21-day cycles of the combination. In certain embodiments, the treatment comprises administering to the patient at least six 21-day cycles of the combination.

In certain aspects, the present disclosure provides a method of treating a patient with DLBCL comprising administering to the patient a therapeutic amount of a combination of:

tafasitamab;

lenalidomide;

rituximab;

cyclophosphamide;

doxorubicin;

vincristine;

prednisone or prednisolone; and

granulocyte colony stimulating factor (G-CSF) or pegylated G-CSF;

wherein the patient has Stage III or Stage IV DLBCL prior to starting the administering.

In certain aspects, the present disclosure concerns a therapeutic combination of: tafasitamab in a 12 mg/kg body weight dose;

rituximab in a 375 mg/m² dose;

cyclophosphamide in a 750 mg/m² dose;

doxorubicin in a 50 mg/m² dose;

vincristine in a 1.4 to 2.0 mg/m² dose; and

prednisone or prednisolone in a 100 mg dose.

In certain aspects, the present disclosure concerns a therapeutic combination of:

tafasitamab in a 12 mg/kg body weight dose;

lenalidomide in a 25 mg dose;

rituximab in a 375 mg/m² dose;

cyclophosphamide in a 750 mg/m² dose;

doxorubicin in a 50 mg/m² dose;

vincristine in a 1.4 to 2.0 mg/m² dose; and

prednisone or prednisolone in a 100 mg dose.

In certain aspects, the present disclosure concerns a therapeutic combination of:

tafasitamab;

rituximab;

cyclophosphamide;

doxorubicin;

vincristine;

prednisone or prednisolone; and granulocyte colony stimulating factor (G-CSF) or pegylated G-CSF.

In certain aspects, the present disclosure concerns a therapeutic combination of:

tafasitamab;

lenalidomide;

rituximab;

cyclophosphamide;

doxorubicin;

vincristine;

prednisone or prednisolone; and granulocyte colony stimulating factor (G-CSF) or pegylated G-CSF.

In certain aspects, administering the combination of the anti-CD19 antibody and R-CHOP is carried out by administering the anti-CD19 antibody and R-CHOP in combination simultaneously. In certain aspects, administering the combination of the anti-CD19 antibody and R-CHOP is carried out by administering the anti-CD19 antibody and R-CHOP consecutively in order. In certain aspects, administering the combination of the anti-CD19 antibody and R-CHOP is carried out by administering the anti-CD19 antibody and R-CHOP consecutively in reverse-order.

In certain aspects, administering the combination of tafasitamab and R-CHOP is carried out by administering the tafasimab and R-CHOP in combination simultaneously. In certain aspects, administering the combination of tafasitamab and R-CHOP is carried out by administering the anti-CD19 antibody and R-CHOP consecutively in order. In certain aspects, administering the combination of tafasitamab and R-CHOP is carried out by administering tafasitamab and R-CHOP consecutively in reverse-order.

In certain aspects, administering the combination of the anti-CD19 antibody, lenalidomide, and R-CHOP is carried out by administering the anti-CD19 antibody, lenalidomide, and R-CHOP in combination simultaneously. In certain aspects, administering the combination of the anti-CD19 antibody, lenalidomide, and R-CHOP is carried out by administering the anti-CD19 antibody, lenalidomide, and R-CHOP consecutively in order. In certain aspects, administering the combination of the anti-CD19 antibody, lenalidomide, and R-CHOP is carried out by administering the anti-CD19 antibody and R-CHOP consecutively in reverse-order.

In certain aspects, administering the combination of tafasitamab, lenalidomide, and R-CHOP is carried out by administering the tafasitamab, lenalidomide, and R-CHOP in combination simultaneously. In certain aspects, administering the combination of tafasitamab, lenalidomide, and R-CHOP is carried out by administering the anti-CD19 antibody and R-CHOP consecutively in order. In certain aspects, administering the combination of tafasitamab, lenalidomide, and R-CHOP is carried out by administering tafasitamab and R-CHOP consecutively in reverse-order.

In certain aspects, the present disclosure provides methods of treating a patient with DLBCL, wherein each dose of tafasitamab is from 8 mg/kg body weight to 40 mg/kg body weight. In certain aspects, the present disclosure provides methods of treating a patient with DLBCL, wherein each dose of tafasitamab is from 500 mg to 3000 mg.

In certain aspects, the present disclosure provides methods of treating a patient with DLBCL, wherein the dose of lenalidomide is 20 mg daily. In certain aspects, the present disclosure provides methods of treating a patient with DLBCL, wherein the dose of lenalidomide is 15 mg daily. In certain aspects, the present disclosure provides methods of treating a patient with DLBCL, wherein the dose of lenalidomide is 15 mg daily.

In certain embodiments, tafasitamab is replaced with an anti-CD19 antibody comprising an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

In certain embodiments, tafasitamab is replaced with an anti-CD19 antibody comprising a variable heavy chain of the sequence

(SEQ ID NO: 7)
EVQLVESGGGLVKPGGSLKLSCAASGYTFTSYVMHVVVRQAPGKGLEW
IGYINPYNDGTKYNEKFQGRVTISSDKSISTAYMELSSLRSEDTAMYY
CARGTYYYGTRVFDYVVGQGTLVTVSS

and a variable light chain of the sequence

(SEQ ID NO: 8)
DIVMTQSPATLSLSPGERATLSCRSSKSLQNVNGNTYLYWFQQKPGQS
PQLLIYRMSNLNSGVPDRFSGSGSGTEFTLTISSLEPEDFAVYYCMQH
LEYPITFGAGTKLEIK.

In certain embodiments of the present disclosure the anti-CD19 antibody replacing tafasitamab is a human, humanized, or chimeric antibody. In another embodiment of the present disclosure the anti-CD19 antibody replacing tafasitamab is of the IgG isotype. In another embodiment the antibody replacing tafasitamab is IgG1, IgG2, or IgG1/IgG2 chimeric. In another embodiment of the present disclosure the isotype of the anti-CD19 antibody replacing tafasitamab is engineered to enhance antibody-dependent cell-mediated cytotoxicity. In another embodiment the heavy chain constant region of the anti-CD19 antibody replacing tafasitamab comprises amino acids 239D and 332E, wherein the Fc numbering is according to the EU index as in Kabat. In another embodiment the anti-CD19 antibody replacing tafasitamab is IgG1, IgG2 or IgG1/IgG2, and the chimeric heavy chain constant region of the anti-CD19 antibody comprises amino acids 239D and 332E, wherein the Fc numbering is according to the EU index as in Kabat.

An anti-CD19 antibody described herein (e.g., tafasitamab) and lenalidomide and rituximab can be used in combination to treat a non-Hodgkin lymphoma in a human subject in need thereof. In some embodiments, the non-Hodgkin lymphoma is selected from the group consisting of follicular lymphoma, small lymphocytic lymphoma, mucosa-associated lymphoid tissue lymphoma, marginal zone lymphoma, diffuse large B cell lymphoma, Burkitt's lymphoma, and mantle cell lymphoma. In some embodiments, the non-Hodgkin lymphoma is relapsed/refractory follicular lymphoma. In some embodiments, the non-Hodgkin lymphoma is relapsed/refractory marginal zone lymphoma

Another aspect comprises a combination of an anti-CD19 antibody described herein (e.g., tafasitamab) and lenalidomide and rituximab for use in the treatment of a non-Hodgkin lymphoma. In some embodiments, the non-Hodgkin lymphoma is selected from the group consisting of follicular lymphoma, small lymphocytic lymphoma, mucosa-associated lymphoid tissue lymphoma, marginal zone lymphoma, diffuse large B cell lymphoma, Burkitt's lymphoma, and mantle cell lymphoma. In some embodiments, the non-Hodgkin lymphoma is relapsed/refractory follicular lymphoma. In some embodiments, the non-Hodgkin lymphoma is relapsed/refractory marginal zone lymphoma

Another aspect comprises a combination of an anti-CD19 antibody described herein (e.g., tafasitamab) and lenalidomide and rituximab in the manufacture of a medicament for treating a non-Hodgkin lymphoma. In some embodiments, the non-Hodgkin lymphoma is selected from the group consisting of follicular lymphoma, small lymphocytic lymphoma, mucosa-associated lymphoid tissue lymphoma, marginal zone lymphoma, diffuse large B cell lymphoma, Burkitt's lymphoma, and mantle cell lymphoma. In some embodiments, the non-Hodgkin lymphoma is relapsed/refractory follicular lymphoma. In some embodiments, the non-Hodgkin lymphoma is relapsed/refractory marginal zone lymphoma

An anti-CD19 antibody described herein (e.g., tafasitamab) and lenalidomide and rituximab can be used in combination to treat chronic lymphocytic leukemia in a human subject in need thereof.

Another aspect comprises a combination of an anti-CD19 antibody described herein (e.g., tafasitamab) and lenalidomide and rituximab for use in the treatment of chronic lymphocytic leukemia.

Another aspect comprises a combination of an anti-CD19 antibody described herein (e.g., tafasitamab) and lenalidomide and rituximab in the manufacture of a medicament for treating chronic lymphocytic leukemia.

An anti-CD19 antibody described herein (e.g., tafasitamab) and lenalidomide and rituximab can be used in combination to treat acute lymphoblastic leukemia in a human subject in need thereof.

Another aspect comprises a combination of an anti-CD19 antibody described herein (e.g., tafasitamab) and lenalidomide and rituximab for use in the treatment of acute lymphoblastic leukemia.

Another aspect comprises a combination of an anti-CD19 antibody described herein (e.g., tafasitamab) and lenalidomide and rituximab in the manufacture of a medicament for treating acute lymphoblastic leukemia.

In specific embodiments of the combinations as described herein, the combination of an anti-CD19 antibody described herein (e.g., tafasitamab) and lenalidomide and rituximab is synergistic. In some embodiments, lenalidomide and/or rituximab is administered prior to administration of the anti-CD19 antibody.

In some embodiments, lenalidomide and/or rituximab is administered after the administration of the anti-CD19 antibody.

In some embodiments, the anti-CD19 antibody and lenalidomide and/or rituximab are administered simultaneously or together.

Methods of assessing clinical responses to treatment are known in the art, and include for example the response assessment criteria based on the Lugano classification (Cheson, 2014; see Appendix D). In some embodiments, administration of the combination of the anti-CD19 antibody and R-CHOP leads to a therapeutic effect selected from the group consisting of an objective response (OR), a partial response (PR) or a complete response (CR). In one embodiment, the therapeutic effect is an objective response (OR). In one embodiment, the therapeutic effect is a partial response (PR). In one embodiment, the therapeutic effect is a complete response (CR).

Therapeutic effects following administration of the combination of the anti-CD19 antibody and R-CHOP may be validated based on the response rate (e.g. the OR rate (ORR), PR rate (PRR) and/or the CR rate (CRR)), the duration of response (DoR) rate, or duration of complete response (DoCR) rate in a population of patients with DLBCL validated based on the response rate (e.g. the OR rate (ORR), PR rate (PRR) and/or the CR rate (CRR)), the duration of response (DoR) rate, or duration of complete response (DoCR) rate in a population of patients with DLBCL. A reference to a therapeutic effect that can be validated based on a response rate in a population can relate to the situation where the therapy has been shown previously to have the response rate specified, e.g. the package insert and/or authorisation for the anti-CD19 antibody may refer to a study showing that response rate in a clinical trial. A reference to a therapeutic effect that can be validated based on a response rate in a population can relate to the situation where the therapy has been shown previously to have the response rate specified, e.g. the package insert and/or authorisation for the anti-CD19 antibody may refer to a study showing that response rate in a clinical trial.

In some embodiments, administration of the combination of the anti-CD19 antibody and R-CHOP leads to an ORR that is at least 70%, 75%, 80%, 85%, 90% or 95% in a population of patients with DLBCL. In some embodiments, administration of the combination of the anti-CD19 antibody and R-CHOP leads to an ORR that is at least 75% in a population of patients with DLBCL. In some embodiments, administration of the combination of the anti-CD19 antibody and R-CHOP leads to a CRR that is at least 50%, 55%, 60%, 65%, 70%, 75%, 80%, 90% or 95% in a population of patients with DLBCL. In some embodiments, administration of the combination of the anti-CD19 antibody and R-CHOP leads to a CRR that is at least 70% in a population of patients with DLBCL.

In some embodiments, administration of the combination of the anti-CD19 antibody and R-CHOP leads to a DoR that is at least 80% in a population of patients with DLBCL. In some embodiments, the DoR is estimated at 6 months following treatment with the combination. In some embodiments, administration of the combination of the anti-CD19 antibody and R-CHOP leads to a DoCR that is at least 80% in a population of patients with DLBCL. In some embodiments, the DoCR is estimated at 6 months following treatment with the combination.

In some embodiments, administration of the combination of the anti-CD19 antibody, R-CHOP and lenalidomide leads to a therapeutic effect selected from the group consisting of an objective response (OR), a partial response (PR) or a complete response (CR). In one embodiment, the therapeutic effect is an objective response (OR). In one embodiment, the therapeutic effect is a partial response (PR). In one embodiment, the therapeutic effect is a complete response (CR).

Therapeutic effects following administration of the combination of the anti-CD19 antibody, R-CHOP and lenalidomide may be validated based on the response rate (e.g. the OR rate (ORR), PR rate (PRR) and/or the CR rate (CRR)), the duration of response (DoR) rate, or duration of complete response (DoCR) rate in a population of patients with DLBCL validated based on the response rate (e.g. the OR rate (ORR), PR rate (PRR) and/or the CR rate (CRR)), the duration of response (DoR) rate, or duration of complete response (DoCR) rate in a population of patients with DLBCL. A reference to a therapeutic effect that can be validated based on a response rate in a population can relate to the situation where the therapy has been shown previously to have the response rate specified, e.g. the package insert and/or authorisation for the anti-CD19 antibody may refer to a study showing that response rate in a clinical trial. A reference to a therapeutic effect that can be validated based on a response rate in a population can relate to the situation where the therapy has been shown previously to have the response rate specified, e.g. the package insert and/or authorisation for the anti-CD19 antibody may refer to a study showing that response rate in a clinical trial.

In some embodiments, administration of the combination of the anti-CD19 antibody, R-CHOP and lenalidomide leads to an ORR that is at least 70%, 75%, 80%, 85%, 90% or 95% in a population of patients with DLBCL. In some embodiments, administration of the combination of the anti-CD19 antibody, R-CHOP and lenalidomide leads to an ORR that is at least 80% in a population of patients with DLBCL. In some embodiments, administration of the combination of the anti-CD19 antibody, R-CHOP and lenalidomide leads to a CRR that is at least 50%, 55%, 60%, 65%, 70%, 75%, 80%, 90% or 95% in a population of patients with DLBCL. In some embodiments, administration of the combination of the anti-CD19 antibody, R-CHOP and lenalidomide leads to a CRR that is at least 65% in a population of patients with DLBCL.

In some embodiments, administration of the combination of the anti-CD19 antibody, R-CHOP and lenalidomide leads to a DoR that is at least 85% in a population of patients with DLBCL. In some embodiments, the DoR is estimated at 6 months following treatment with the combination. In some embodiments, administration of the combination of the anti-CD19 antibody, R-CHOP and lenalidomide leads to a DoCR that is at least 95% in a population of patients with DLBCL. In some embodiments, the DoCR is estimated at 6 months following treatment with the combination.

### Patients

The present disclosure provides a therapeutic combination comprising tafasitamab and R-CHOP or tafasitamab, lenalidomide, and R-CHOP for use in the treatment of patients with Diffuse Large B-cell Lymphoma (DLBCL).

In certain embodiments, the patients with DLBCL have an International Prognostic Index (IPI) status of 2-5, 3-5, 4-5, 3-4, 3, 4, or 5 prior to starting the administering. In certain embodiments, the patients with DLBCL have Stage III or Stage IV DLBCL prior to starting the administering. In certain embodiments, the patients with DLBCL have an International Prognostic Index (IPI) status of 2-5, 3-5, 4-5, 3-4, 3, 4, or 5 and Stage III or Stage IV DLBCL prior to starting the administering.

In certain embodiments, the patients with DLBCL are patients with previously untreated DLBCL. In a certain embodiments, the patients with previously untreated DLBCL have an International Prognostic Index (IPI) status of 2-5, 3-5, 4-5, 3-4, 3, 4, or 5 prior to starting the administering. In certain embodiments, the patients with previously untreated DLBCL have Stage III or Stage IV DLBCL prior to starting the administering. In certain embodiments, the patients with previously untreated DLBCL have an International Prognostic Index (IPI) status of 2-5, 3-5, 4-5, 3-4, 3, 4, or 5 and Stage III or Stage IV DLBCL prior to starting the administering. In certain embodiments, the patients with DLBCL are patients with newly diagnosed, previously untreated, high-intermediate or high-risk DLBCL.

In certain embodiments, a patient that is treated has one or more of the following criteria:

1. Age ≥18 years

2. Written informed consent

3. Previously untreated, newly diagnosed and histologically confirmed DLBCL, NOS

4. Tumor tissue for retrospective central pathology review and correlative studies must be provided as an adjunct to participation in this study.

5. Patients must have at least one measurable disease site. The lesion must have a greatest transverse diameter of ≥1.5 cm and greatest perpendicular diameter of ≥1.0 cm at screening. The lesion must be confirmed to be PET-positive at the latest at the time of randomization.

6. Eastern Cooperative Oncology Group (ECOG) performance status of 0 to 2

7. International Prognostic Index (IPI) status of 2 to 5

8. Appropriate candidate for R-CHOP.

9. Left ventricular ejection fraction (LVEF) of ≥50%, assessed by echocardiography or cardiac multi-gated acquisition (MUGA) scan

10. Patient must have the following laboratory criteria at screening:

a. Absolute neutrophil count (ANC) ≥1.5×10⁹/L (unless secondary to bone marrow involvement by DLBCL as demonstrated by recent bone marrow aspiration and bone marrow biopsy)

b. Platelet count ≥75×10⁹/L (unless secondary to bone marrow involvement by DLBCL as demonstrated by recent bone marrow aspiration and bone marrow biopsy)

c. Total serum bilirubin ≤1.5× upper limit of normal (ULN) unless secondary to Gilbert's syndrome or documented liver involvement by lymphoma. Patients with Gilbert's syndrome or documented liver involvement by lymphoma may be included if their total bilirubin is ≤5×ULN

d. Alanine transaminase (ALT), aspartate aminotransferase (AST) and alkaline phosphatase (ALP) ≤3× ULN, or <5× ULN in cases of documented liver involvement

e. Serum creatinine clearance

(all countries except US:) must be ≥50 mL/minute either measured or calculated using a standard Cockcroft and Gault formula (Cockroft, 1976)

(US only:) must be ≥60 mL/minute either measured or calculated using a standard Cockcroft and Gault formula (Cockroft, 1976)

11. Females of childbearing potential (FCBP) must:

Applicable in All Countries Except US:

a. not be pregnant as confirmed by a negative serum pregnancy test at screening and a medically supervised urine pregnancy test prior to starting study therapy

b. refrain from breast feeding and donating oocyte during the course of study and for 3 months after the last dose of study drug or, for R-CHOP, according to the local guidelines, whichever is longer.

c. agree to ongoing pregnancy testing during the course of the study, and after study therapy has ended. This applies even if the patient applies complete sexual abstinence

d. commit to continued abstinence from heterosexual intercourse if it is in accordance with her lifestyle (which must be reviewed on a monthly basis) or agree to use and be able to comply with the use of highly effective contraception without interruption at least 4 weeks prior to start of study drugs, during the study treatment and for 3 months after the last dose of study drug, or, for R-CHOP, according to the local guidelines, whichever is longer. Please refer to section 7.3.1

Applicable in US:

a. not be pregnant as confirmed by pregnancy tests performed before treatment initiation, within 10-14 days and again within 24 hours of initiating treatment (even if true abstinence is the chosen method of birth control).

b. refrain from breast feeding and donating oocytes during the course of study and for 3 months after the last dose of study drug or, for R-CHOP, according to the US guidelines, whichever is longer.

c. agree to ongoing pregnancy testing during the course of the study (every 3 weeks in women with regular menstrual cycle and every 2 weeks in women with irregular menstrual cycle), and after study therapy has ended (even if true abstinence is the chosen method of birth control).

d. not get pregnant while taking the study drug and for at least 3 months after stopping the study drug by using at the same time 2 effective methods of contraception (at least one highly effective method and one additional effective method) each time engaging in sexual activity with a male, starting at least 4 weeks before taking the study drug, while taking the study drug, during breaks (dose interruptions) and for at least 3 months after stopping the study drug or, for R-CHOP, according to the US guidelines, whichever is longer. True abstinence from heterosexual sexual intercourse is also an acceptable method of contraception. The use of emergency contraception is also permitted.

12. Male participants must:

Applicable in All Countries Except US:

Use an effective barrier method of contraception without interruption if the patient is sexually active with female of childbearing potential (FCBP) . Male participants should refrain from donating sperm during the study participation and for 3 months after the last dose of study drug, or, for R-CHOP, according to the local guidelines, whichever is longer.

Applicable in US:

Use latex or synthetic condom each time they have sex with a woman of childbearing potential. True abstinence from heterosexual sexual intercourse is also an acceptable method of contraception. The use of emergency contraception is also permitted. Male participants should refrain from donating sperm during the study participation and for 3 months after the last dose of study drug, or, for R-CHOP, according to the US guidelines, whichever is longer.

13. In the opinion of investigator, the patient must:

a. be able and willing to receive adequate prophylaxis and/or therapy for thromboembolic events, e.g. aspirin 70-325 mg daily or low molecular weight heparin. This is due to increased risk of thrombosis in patients treated with lenalidomide without prophylaxis. Patients unable or unwilling to take any prophylaxis are not eligible

b. be able to understand, give written informed consent, and comply with all study-related procedures, medication use, and evaluations

c. not have a history of noncompliance in relation to medical regimens or be considered potentially unreliable and/or uncooperative

d. be able to understand the reason for complying with the special conditions of the pregnancy prevention risk management plan and give written acknowledgement of this.

In certain embodiments, the patient is excluded from treatment based on one or more of the following criteria:

1. Any other histological type of lymphoma according to WHO2016 classification of lymphoid neoplasms, e.g. primary mediastinal (thymic) large B-cell (PMBL), known double- or triple-hit lymphoma or Burkitt's lymphoma.

2. Transformed NHL and/or evidence of composite lymphoma

3. History of radiation therapy to ≥25% of the bone marrow for other diseases or history of anthracycline therapy

4. History of prior non-hematologic malignancy except for the following:

a. Malignancy treated with curative intent and with no evidence of active disease present for more than 2 years before screening

b. Adequately treated lentigo maligna melanoma without current evidence of disease or adequately controlled non-melanomatous skin cancer.

c. Adequately treated carcinoma in situ without current evidence of disease.

5. History of myocardial infarction 56 months, or congestive heart failure requiring use of ongoing maintenance therapy for life-threatening arrhythmias.

6. Patients with:

a. Known positive test result for hepatitis C (hepatitis C virus [HCV] antibody serology testing) and a positive test for HCV RNA. Patients with positive serology must have been tested locally for HCV RNA and are eligible, in case of negative HCV RNA test results.

b. Known positive test results for chronic HBV infection (defined by HBsAg positivity). Patients with occult or prior HBV infection (defined as negative HBsAg and positive total HBcAb) may be included if HBV DNA was undetectable (local test result), provided that they are willing to undergo ongoing DNA testing. Antiviral prophylaxis may be administered as per institutional guidelines. Patients who have protective titers of hepatitis B surface antibody (HBsAb) after vaccination or prior but cured hepatitis B are eligible.

c. Known seropositive for or history of active viral infection with human immunodeficiency virus (HIV)

d. Known active bacterial, viral, fungal, mycobacterial, or other infection at screening.

e. Known CNS lymphoma involvement

f. History or evidence of clinically significant cardiovascular, CNS and/or other systemic disease that would in the investigator opinion preclude participation in the study or compromise the patient's ability to give informed consent

g. History or evidence of rare hereditary problems of galactose intolerance, Lapp lactase deficiency or glucose-galactose malabsorption

h. Vaccination with live vaccine within 21 days prior to study randomization

i. Major surgery (excluding lymph node biopsy) within up to 21 days prior to signing the informed consent form, unless the patient is recovered at the time of signing the informed consent form

j. Any anti-cancer and/or investigational therapy within 21 days prior to the start of Cycle 1. Note: Steroid pre-phase is permitted

k. Pregnancy or lactation

l. History of hypersensitivity to any component of R-CHOP, to lenalidomide, to compounds of similar biological or chemical composition as tafasitamab, IMiDs® and/or the excipients contained in the study drug formulations or R-CHOP

m. Any contraindication concerning any individual component of R-CHOP

In certain embodiments, a patient that is treated has one or more of the following criteria:

1. Written informed consent.

2. Age 18 to 80 years at the time of signing the ICF.

3. Previously untreated patients with local biopsy-proven, CD20-positive DLBCL, including one of the following diagnoses by 2016 WHO classification of lymphoid neoplasms are eligible (Swerdlow et al., 2016):

a. DLBCL, not otherwise specified (NOS) including germinal center B-cell (GCB) type, activated B-cell (ABC) type

b. T-cell rich large BCL

c. Epstein-Barr virus-positive DLBCL, NOS

d. Anaplastic lymphoma kinase (ALK)-positive large BCL

e. Human Herpes virus-8 (HHV8)-positive DLBCL, NOS

f. High-grade BCL with MYC and B-cell lymphoma 2 (BCL2) and/or B-cell lymphoma 6 (BCL6) rearrangements (double-hit or triple-hit lymphoma). Please note: Patients must be appropriate candidates for R-CHOP. If an investigator deems a patient with a known double- or triple-hit lymphoma (HGBL) should be treated more aggressively (e.g. dose-adjusted etoposide, prednisone, vincristine, cyclophosphamide, doxorubicin and rituximab [DA-EPOCH-R] or cyclophosphamide, vincristine, doxorubicin and dexamethasone (CVAD) followed by methotrexate and cytarabine [Hyper CVAD]), this patient would not be considered eligible for this study

g. DLBCL coexistent with either follicular lymphoma (FL) of any grade, gastric MALT lymphoma or non-gastric MALT lymphoma

h. FL grade 3b

4. Availability of archival or freshly collected tumor tissue sent for retrospective central pathology review. Please note: neither receipt of tumor samples nor central review of diagnosis is necessary prior to study enrollment.

5. Up to six of the largest target nodes, nodal masses, or other lymphomatous lesions that are measurable in two diameters should be identified by local assessment from different body regions representative of the patient's overall disease burden and include mediastinal and retroperitoneal disease, if involved. At baseline, a measurable node must be greater than 15 mm in longest diameter (LDi). Measurable extranodal disease may be included in the six representative, measured lesions. At baseline, measurable extranodal lesions should be greater than 10 mm LDi. All other lesions (including nodal, extranodal, and assessable disease) should be followed as nonmeasured disease as non-target lesions (e.g. cutaneous, GI, spleen, liver, kidneys, pleural or pericardial effusions, ascites, bone, bone marrow). At least one measurable lesion must be confirmed to be PET-positive (Deauville score of 4 or 5) at the time of randomization by local assessment.

6. ECOG performance status of 0, 1, or 2.

7. IPI status of 3 to 5 (for patients >60 years of age) or aaIPI 2 to3 (for patients ≤60 years of age).

8. Diagnosis to treatment interval, defined as the time between the date of DLBCL diagnosis (date of the first biopsy specimen containing lymphoma according to the local pathology report) and the start of treatment (C1D1) ≤28 days.

9. Left ventricular ejection fraction equal to or greater than lower limit of institutional normal range, assessed by local echocardiography or cardiac multi-gated acquisition (MUGA) scan.

10. Patient must have the following local laboratory criteria at screening:

a. Absolute neutrophil count (ANC) ≥1.5×10⁹/L (unless secondary to bone marrow involvement by DLBCL)

b. Platelet count ≥75×10⁹/L (unless secondary to bone marrow involvement by DLBCL)

c. Total serum bilirubin <1.5 x upper limit of normal (ULN) unless secondary to Gilbert's Syndrome or documented liver involvement by lymphoma. Patients with Gilbert's Syndrome or documented liver involvement by lymphoma may be included if their total bilirubin is ≤5×ULN

d. Alanine aminotransferase (ALT), aspartate aminotransferase (AST) and alkaline phosphatase (ALP) ≤3×ULN, or ≤5×ULN in cases of documented liver involvement

e. Serum creatinine clearance must be ≥30 mL/minute either measured or calculated using a standard Cockcroft and Gault formula (Cockroft and Gault, 1976)

11. In the opinion of investigator, the patient must:

a. Be able and willing to receive adequate prophylaxis and/or therapy for thromboembolic events, e.g. aspirin 81 to 325 mg daily or low molecular weight heparin. This is due to increased risk of thrombosis in patients treated with Ienalidomide without prophylaxis. Patients unable or unwilling to take any prophylaxis are not eligible

b. Be able to understand, give written informed consent, and comply with all study-related procedures, medication use, and evaluations

c. Not have a history of noncompliance in relation to medical regimens nor be considered potentially unreliable and/or uncooperative

d. Be able to understand the reason for complying with the special conditions of the pregnancy prevention risk management plan and in writing acknowledge to adhere to this plan

12. Due to the teratogenic potential of lenalidomide, females of childbearing potential (FCBP) must:

Applicable in All Countries Except US:

a. Not be pregnant as confirmed by a negative serum pregnancy test at screening and a medically supervised urine pregnancy test prior to starting study therapy

b. Refrain from breast feeding and donating oocytes during the course of study and for 3 months after the last dose of study drug or according to local guidelines for R-CHOP, whichever is longer

c. Agree to ongoing pregnancy testing during the course of the study and after study therapy has ended. This applies even if the patient applies complete sexual abstinence

d. Commit to continued abstinence from heterosexual intercourse if it is in accordance with her lifestyle (which must be reviewed on a monthly basis) or agree to use and be able to comply with the use of highly effective contraception without interruption at least 4 weeks prior to start of study drugs, during the study treatment and for 3 months after the last dose of study drug, or, for R-CHOP, according to the local guidelines, whichever is longer.

Applicable in US:

e. Not be pregnant as confirmed by pregnancy tests performed before treatment initiation, within 10-14 days and again within 24 hours of initiating treatment (even if true abstinence is the chosen method of birth control)

f. Refrain from breast feeding and donating oocytes during the course of study and for 3 months after the last dose of study drug, or according to US guidelines for R-CHOP, whichever takes longer

g. Agree to ongoing pregnancy testing during the course of the study (every 3 weeks in women with regular menstrual cycle and every 2 weeks in women with irregular menstrual cycle), and after study therapy has ended (even if true abstinence is the chosen method of birth control)

h. Not get pregnant while taking the study drug and for at least 3 months after the last dose of study drugs by using at the same time 2 effective methods of contraception, each time engaging in sexual activity with a male, starting at least 4 weeks before taking the study drug, while taking the study drug, during breaks (dose interruptions) and for at least 3 months after stopping the study drug, or for R-CHOP, according to the US guidelines, whichever is longer. True abstinence from heterosexual sexual intercourse is also an acceptable method of contraception. The use of emergency contraception is also permitted.

13. Male participants must:

Applicable in All Countries Except US:

a. Use an effective barrier method of contraception without interruption if the patient is sexually active with FCBP. Male participants should refrain from donating sperm during the study participation and for 3 months after the last dose of study drug, or according to the local guidelines for R-CHOP, whichever is longer

Applicable in US:

b. Use a latex or synthetic condom each time they have sex with a FCBP. True abstinence from heterosexual sexual intercourse is also an acceptable method of contraception. The use of emergency contraception is also permitted. Male participants should refrain from donating sperm during the study participation and for 3 months after the last dose of study drug, or according to the US guidelines for R-CHOP, whichever is longer

In certain embodiments, the patient is excluded from treatment based on one or more of the following criteria:

1. Any other histological type of lymphoma according to WHO 2016 classification of lymphoid neoplasms, e.g. primary mediastinal (thymic) large B-cell lymphoma, Burkitt's lymphoma, BCL, unclassifiable, with features intermediate between DLBCL and classical Hodgkin lymphoma (grey-zone lymphoma); primary effusion lymphoma; primary cutaneous DLBCL, leg type; primary DLBCL of the CNS; DLBCL arising from CLL or indolent lymphoma.

2. History of radiation therapy to ≥25% of the bone marrow for other diseases.

3. History of prior non-hematologic malignancy except for the following:

a. Malignancy treated with curative intent and with no evidence of active disease present for more than 2 years before screening

b. Adequately treated lentigo maligna melanoma without current evidence of disease or adequately controlled non-melanomatous skin cancer.

c. Adequately treated carcinoma in situ without current evidence of disease

4. Patients with:

a. Positive local test result during screening for hepatitis C (hepatitis C virus [HCV] antibody serology testing) and a positive test for HCV RNA. Patients with positive serology must have been tested locally for HCV RNA and are eligible, in case of negative HCV RNA test results

b. Positive local test result during screening for chronic hepatitis B virus (HBV) infection (defined by hepatitis B surface antigen [HBsAg] positivity). Patients with occult or prior HBV infection (defined as negative HBsAg and positive total hepatitis B core antibody [HBcAb]) may be included if HBV DNA was undetectable (local test result), provided that they are willing to undergo ongoing DNA testing. Antiviral prophylaxis may be administered as per institutional guidelines. Patients who have protective titers of hepatitis B surface antibody (HBsAb) after vaccination or prior but cured hepatitis B are eligible

c. Seropositive (local test during screening) for, or history of active viral infection with human immunodeficiency virus (HIV)

d. Known active systemic bacterial, viral, fungal, or other infection at screening, including patients with suspected active or latent tuberculosis (as confirmed by a positive interferon-gamma release assay)

e. Positive results for the human T-lymphotrophic 1 virus (HTLV-1). HTLV testing during screening is required for patients at sites in endemic countries (Japan and Melanesia and countries in the Caribbean basin, South America, Central America, and sub-Saharan Africa)

f. Known CNS lymphoma involvement

g. History or evidence of clinically significant cardiovascular, CNS and/or other systemic disease that would in the investigator's opinion preclude participation in the study or compromise the patient's ability to give informed consent

h. History or evidence of rare hereditary problems of galactose intolerance, Lapp lactase deficiency or glucose-galactose malabsorption

i. Vaccination with live vaccine within 21 days prior to study randomization

j. Major surgery within up to 21 days prior to signing the ICF, unless the patient is recovered at the time of signing the ICF

k. Any systemic anti-lymphoma and/or investigational therapy prior to the start of C1D1, except for permitted pre-phase treatment.

l. Contraindication to any of the individual components of R-CHOP, including prior receipt of anthracyclines

m. Pregnancy or lactation

n. History of hypersensitivity to any component of R-CHOP, to lenalidomide, to compounds of similar biological or chemical composition to tafasitamab, IMiDs® and/or the excipients contained in the study drug formulations

International Prognostic Index (IPI)

In certain aspects, the present disclosure provides a DLBCL risk assessment using the International Prognostic Index (IPI) for predicting outcomes. An IPI assessment includes consideration of the following five risk factors:

Ann Arbor Stage III or IV DLBCL;

Age greater than or equal to 60 years;

Serum LDH greater than 1× upper limit of normal (ULN);

Eastern Cooperative Oncology Group (ECOG) performance status greater than or equal to 2; and

Extranodal involvement greater than or equal to 2 (extranodal involvement per Cheson 2014 can include sites that have a focal uptake by positron emission tomography (PET)-CT (e.g., spleen, liver, bone, thyroid, cutaneous, gastrointestinal (GI), kidneys, pleural or pericardial effusions, ascities).

A patient with zero or one risk factor is considered to be in an IPI low risk group. A patient with two risk factors is considered to be in an IPI low-intermediate risk group. A patient with three risk factors is considered to be in an IPI high-intermediate risk group. A patient with four or five risk factors is considered to be in an IPI high risk group.

A higher IPI score is predictive of a worse outcome compared to a lower IPI score, and treatment of patients with higher IPI scores typically is less successful than treating patients with lower IPI scores.

Antibody Sequences

TABLE 1

| | SEQ ID NO: | Amino Acids |
|---|---|---|
| HCDR1 | SEQ ID NO: 1 | SYVMH |
| HCDR2 | SEQ ID NO: 2 | NPYNDG |
| HCDR3 | SEQ ID NO: 3 | GTYYYGTRVFDY |
| LCDR1 | SEQ ID NO: 4 | RSSKSLQNVNGNTYLY |
| LCDR2 | SEQ ID NO: 5 | RMSNLNS |
| LCDR3 | SEQ ID NO: 6 | MQHLEYPIT |
| VH | SEQ ID NO: 7 | EVQLVESGGGLVKPGGSLKLSCAASGYTFTSYVMH |
| | | VVVRQAPGKGLEWIGYINPYNDGTKYNEKFQGRVTIS |
| | | SDKSISTAYMELSSLRSEDTAMYYCARGTYYYGTRV |
| | | FDYWG QGTLVTVSS |
| VL | SEQ ID NO: 8 | DIVMTQSPATLSLSPGERATLSCRSSKSLQNVNGNT |
| | | YLYWFQQKPGQSPQLLIYRMSNLNSGVPDRFSGSG |
| | | SGTEFTLTISSLEPEDFAVYYCMQHLEYPITFGAGTKL |
| | | EIK |
| Heavy chain constant | SEQ ID NO: 9 | ASTKGPSVFPLAPSSKSTSGGTAALGCLVKDYFPEP |
| domain | | VTVSWNSGALTSGVHTFPAVLQSSGLYSLSSVVTVP |
| | | SSSLGTQTYICNVNHKPSNTKVDKKVEPKSCDKTHT |
| | | CPPCPAPELLGGPDVFLFPPKPKDTLMISRTPEVTCV |
| | | VVDVSHEDPEVQFNVVYVDGVEVHNAKTKPREEQFN |
| | | STFRWSVLTWHQDWLNGKEYKCKVSNKALPAPEE |
| | | KTISKTKGQPREPQVYTLPPSREEMTKNQVSLTCLVK |
| | | GFYPSDIAVEWESNGQPENNYKTTPPMLDSDGSFFL |
| | | YSKLTVDKSRWQQGNVFSCSVMHEALHNHYTQKSL |
| | | SLSPGK |
| Light chain constant | SEQ ID NO: 10 | RTVAAPSVFIFPPSDEQLKSGTASVVCLLNNFYPREA |
| domain | | KVQWKVDNALQSGNSQESVTEQDSKD |
| | | STYSLSSTLTLSKADYEKHKVYACEVTHQGLSSPVTK |
| | | SFNRGEC |
| Full Heavy chain | SEQ ID NO: 11 | EVQLVESGGGLVKPGGSLKLSCAASGYTFTSYVMH |
| | | VVVRQAPGKGLEWIGYINPYNDGTKYNEKFQGRVTIS |
| | | SDKSISTAYMELSSLRSEDTAMYYCARGTYYYGTRV |
| | | FDYVVGQGTLVTVSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVD |
| | | KKVEPKSCDKTHTCPPCPAPELLGGPDVFLFPPKPK |
| | | DTLMISRTPEVTCVWDVSHEDPEVQFNVVYVDGVEV |
| | | HNAKTKPREEQFNSTFRVVSVLTVVHQDWLNGKEY |
| | | KCKVSNKALPAPEEKTISKTKGQPREPQVYTLPPSRE |
| | | EMTKNQVSLTCLVKGFYPSDIAVEWESNGQPENNYK |
| | | TTPPMLDSDGSFFLYSKLTVDKSRWQQGNVFSCSV |
| | | MHEALHNHYTQKSLSLSPGK |
| Full Light chain | SEQ ID NO: 12 | DIVMTQSPATLSLSPGERATLSCRSSKSLQNVNGNT |
| | | YLYVVFQQKPGQSPQLLIYRMSNLNSGVPDRFSGSG |
| | | SGTEFTLTISSLEPEDFAVYYCMQHLEYPITFGAGTKL |
| | | EIKRTVAAPSVFIFPPSDEQLKSGTASWCLLNNFYPR |
| | | EAKVQWKVDNALQSGNSQESVTEQDSKDSTYSLSS |
| | | TLTLSKADYEKHKVYACEVTHQGLSSPVTKSFNRGE |
| | | C |

### EXAMPLE 1

Phase Ib, Open-Label, Randomized Study to Assess Safety and Preliminary Efficacy of Tafasitamab in Addition to R-CHOP or Tafasitamab Plus Lenalidomide in Addition to R-CHOP in Patients with Newly Diagnosed Diffuse Large B-Cell Lymphoma (DLBCL)—First-MIND

This open label, prospective, randomized phase Ib study is designed to confirm the safety and preliminary efficacy of tafasitamab in addition to R-CHOP or tafasitamab plus lenalidomide in addition to R-CHOP in patients with newly diagnosed DLBCL.

### 1. Clinical Trial Design

### 1.1 Overall Clinical Trial Design and Investigational Plan

This is a multicenter, open-label, randomized, Phase Ib trial to assess safety and preliminary efficacy of tafasitamab in addition to R-CHOP or tafasitamab plus lenalidomide in addition to R-CHOP in adult patients with newly diagnosed DLBCL.

### Safety Monitoring

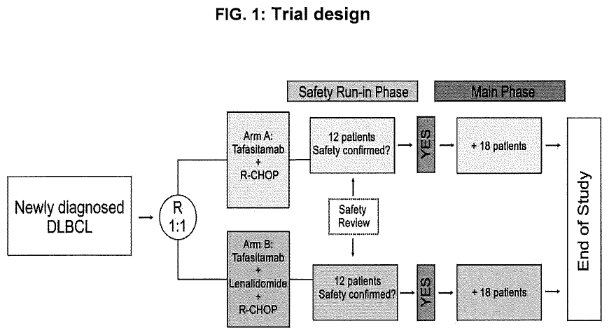

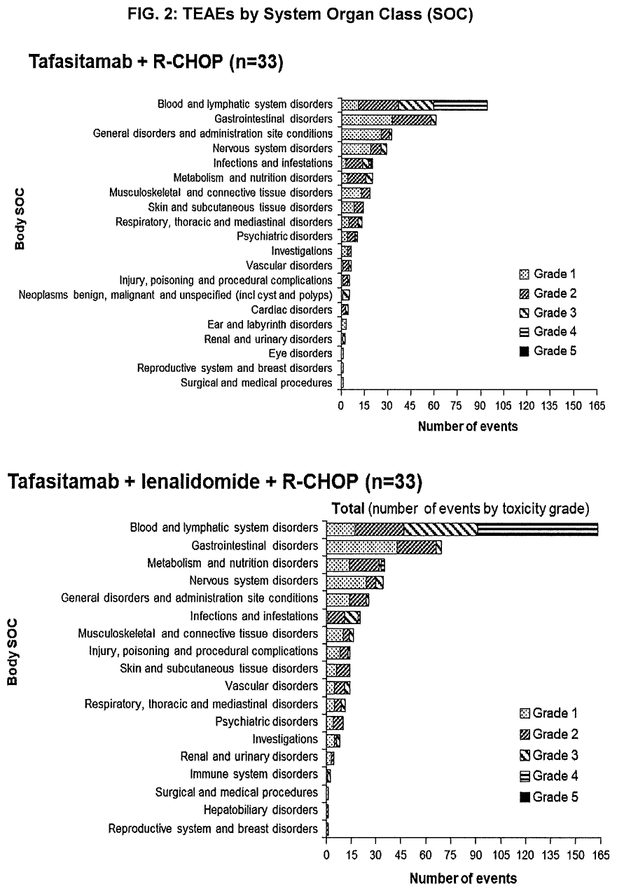

The trial consists of two phases as shown in FIG. 1.

Safety Run-in Phase:

As the addition of tafasitamab to R-CHOP and tafasitamab and lenalidomide plus R-CHOP has not previously been evaluated in a clinical study, a safety run-in phase with 12 patients in each arm will be performed. In order to evaluate the safety in accordance with the stopping rules, enrolment may be paused when 12 patients in each arm have been recruited and have been followed for 21 days after C1D1.

Main Phase:

If no unexpected safety signals (except for those being causally related to R-CHOP) are observed in either arm, enrolment will continue as planned to enrol additional approximately 18 patients in each arm in the main phase.

All patients are expected to receive 6 cycles of study treatment (each cycle consisting of 21 days) and to be followed up for 24 months (or 731 days) from the date of randomization. End of Study

The end of study is defined as the timepoint when data collection will stop and the final analysis of the study will occur. The end of study will happen after all patients have completed their End of Study/Early Follow-up Termination Visit.

### 1.2 Clinical Trial Duration

From the time of providing informed consent, each patient is expected to be included in the study for a duration of approximately 25 months. Three periods are defined for each patient in the study.

Screening Period

The screening period of a maximum of 21 days is the interval between the date of signing of informed consent and the date of randomization.

During screening, each patient who signs the ICF will be allocated a unique identification number. All patients who fulfil all inclusion criteria and who are not barred by any of the exclusion criteria will be randomly assigned to treatment comprising tafasitamab in addition to R-CHOP or tafasitamab plus lenalidomide in addition to R-CHOP in a 1:1 ratio.

Study treatment should start within 24 hours after randomization.

Treatment Period

The treatment period starts with the first administration of study drug (C1D1) and consists of 6 cycles, each 21 days. The End of Treatment Visit or Early Study Treatment Discontinuation Visit will be performed 6±2 weeks after End of Treatment. End of Treatment is defined as day 21 of the last treatment cycle the patient started. Patients who discontinue early because of progression/relapse of disease may have the Early Study Treatment Discontinuation Visit earlier at the discretion of the investigator.

Follow-Up Period

The Follow-up period starts at the End of Treatment or Early Study Treatment Discontinuation Visit; the 30-day safety follow-up visit will be included in this visit. Clinical evaluation will be performed every 3 months. CT scans will be performed every 6 months until final completion of study or until disease progression/relapse. All patients are expected to be followed up for a total of 18 months after the End of Treatment Visit or Early Study Treatment Discontinuation Visit. The End of Study Visit or Early Follow-up Termination Visit marks the completion of the study for an individual patient.

### 1.3 Risks and Benefits to Patients

All eligible patients will be treated with standard of care for six cycles of R-CHOP as a potentially curative treatment approach. In addition, patients will receive either tafasitamab (Arm A) or tafasitamab plus lenalidomide (Arm B) as an add-on to R-CHOP to potentially improve the CR rate and hence to possibly reduce the treatment failure rate. Based on the safety profile of tafasitamab single agent and the well manageable safety profile of the tafasitamab plus lenalidomide combination (as demonstrated in the L-MIND study in R/R DLBCL), it is expected that the overall risk-benefit is favorable without additional toxicities compared to the treatment with R-CHOP only.

The predictable risks and most common side effects of tafasitamab+/−lenalidomide are infusion-related reactions, transient neutropenia, thrombocytopenia, anemia, diarrhea, pyrexia and asthenia. Treatment-related serious AEs consist mainly of infections or neutropenic fever.

Together, the potential risks identified with tafasitamab+/−lenalidomide alongside with the measures in place to minimize risk to patients participating in this trial are justified by the anticipated benefits that may be achieved by the add-on treatment in patients with newly diagnosed DLBCL, which constitutes a life-threatening condition.

### 2. Selection and Withdrawal of Patients

The investigator or designee must ensure that only patients who meet all the following inclusion and none of the exclusion criteria are enrolled in the study.

The patients are not allowed to participate in additional parallel investigational drug or device studies.

The sponsor is not providing waivers to the clinical trial protocol as deviations might have a negative impact on patient safety or the scientific integrity and regulatory acceptability of the clinical trial.

### 2.1 Inclusion Criteria

Patients considered for participation in the clinical trial must meet all of the following criteria:
1. Age >18 years
2. Written informed consent
3. Previously untreated, newly diagnosed and histologically confirmed DLBCL, NOS
4. Tumor tissue for retrospective central pathology review and correlative studies must be provided as an adjunct to participation in this study.
5. Patients must have at least one measurable disease site. The lesion must have a greatest transverse diameter of z1.5 cm and greatest perpendicular diameter of ≥1.0 cm at screening. The lesion must be confirmed to be PET-positive at the latest at the time of randomization.
6. Eastern Cooperative Oncology Group (ECOG) performance status of 0 to 2
7. International Prognostic Index (IPI) status of 2 to 5
8. Appropriate candidate for R-CHOP.
9. Left ventricular ejection fraction (LVEF) of ≥50%, assessed by echocardiography or cardiac multi-gated acquisition (MUGA) scan
10. Patient must have the following laboratory criteria at screening:
a. Absolute neutrophil count (ANC) ≥1.5×10⁹/L (unless secondary to bone marrow involvement by DLBCL as demonstrated by recent bone marrow aspiration and bone marrow biopsy)
b. Platelet count 75×10⁹/L (unless secondary to bone marrow involvement by DLBCL as demonstrated by recent bone marrow aspiration and bone marrow biopsy)
c. Total serum bilirubin ≤1.5× upper limit of normal (ULN) unless secondary to Gilbert's syndrome or documented liver involvement by lymphoma. Patients with Gilbert's syndrome or documented liver involvement by lymphoma may be included if their total bilirubin is ≤5×ULN
d. Alanine transaminase (ALT), aspartate aminotransferase (AST) and alkaline phosphatase (ALP) ≤5×ULN, or <5×ULN in cases of documented liver involvement
e. Serum creatinine clearance
(all countries except US:) must be ≥50 mL/minute either measured or calculated using a standard Cockcroft and Gault formula (Cockroft, 1976)
(US only:) must be ≥60 mL/minute either measured or calculated using a standard Cockcroft and Gault formula (Cockroft, 1976)
11. Females of childbearing potential (FCBP) must:

Applicable in All Countries Except US:
a. not be pregnant as confirmed by a negative serum pregnancy test at screening and a medically supervised urine pregnancy test prior to starting study therapy
b. refrain from breast feeding and donating oocyte during the course of study and for 3 months after the last dose of study drug or, for R-CHOP, according to the local guidelines, whichever is longer.
c. agree to ongoing pregnancy testing during the course of the study, and after study therapy has ended. This applies even if the patient applies complete sexual abstinence
d. commit to continued abstinence from heterosexual intercourse if it is in accordance with her lifestyle (which must be reviewed on a monthly basis) or agree to use and be able to comply with the use of highly effective contraception without interruption at least 4 weeks prior to start of study drugs, during the study treatment and for 3 months after the last dose of study drug, or, for R-CHOP, according to the local guidelines, whichever is longer. Please refer to section 7.3.1

Applicable in US:
e. not be pregnant as confirmed by pregnancy tests performed before treatment initiation, within 10-14 days and again within 24 hours of initiating treatment (even if true abstinence is the chosen method of birth control).
f. refrain from breast feeding and donating oocytes during the course of study and for 3 months after the last dose of study drug or, for R-CHOP, according to the US guidelines, whichever is longer.
g. agree to ongoing pregnancy testing during the course of the study (every 3 weeks in women with regular menstrual cycle and every 2 weeks in women with irregular menstrual cycle), and after study therapy has ended (even if true abstinence is the chosen method of birth control).
h. not get pregnant while taking the study drug and for at least 3 months after stopping the study drug by using at the same time 2 effective methods of contraception (at least one highly effective method and one additional effective method) each time engaging in sexual activity with a male, starting at least 4 weeks before taking the study drug, while taking the study drug, during breaks (dose interruptions) and for at least 3 months after stopping the study drug or, for R-CHOP, according to the US guidelines, whichever is longer. True abstinence from heterosexual sexual intercourse is also an acceptable method of contraception. The use of emergency contraception is also permitted.
12. Male participants must:

Applicable in All Countries Except US:
Use an effective barrier method of contraception without interruption if the patient is sexually active with female of childbearing potential (FCBP). Male participants should refrain from donating sperm during the study participation and for 3 months after the last dose of study drug, or, for R-CHOP, according to the local guidelines, whichever is longer.

Applicable in US:
Use latex or synthetic condom each time they have sex with a woman of childbearing potential. True abstinence from heterosexual sexual intercourse is also an acceptable method of contraception. The use of emergency contraception is also permitted. Male participants should refrain from donating sperm during the study participation and for 3 months after the last dose of study drug, or, for R-CHOP, according to the US guidelines, whichever is longer.
13. In the opinion of investigator, the patient must:
a. be able and willing to receive adequate prophylaxis and/or therapy for thromboembolic events, e.g. aspirin 70-325 mg daily or low molecular weight heparin. This is due to increased risk of thrombosis in patients treated with lenalidomide without prophylaxis. Patients unable or unwilling to take any prophylaxis are not eligible
b. be able to understand, give written informed consent, and comply with all study-related procedures, medication use, and evaluations
c. not have a history of noncompliance in relation to medical regimens or be considered potentially unreliable and/or uncooperative
d. be able to understand the reason for complying with the special conditions of the pregnancy prevention risk management plan and give written acknowledgement of this.

### 2.2 Exclusion Criteria

### 3. Study Treatment

For the purpose of this protocol the following definitions apply:

Study drug shall be used synonymously with Investigational Medicinal Product. Study drugs are tafasitamab and lenalidomide.

Study treatment is defined as tafasitamab in addition to R-CHOP (Arm A) or tafasitamab plus lenalidomide in addition to R-CHOP (Arm B).

Study treatment consists of tafasitamab in addition to six cycles of R-CHOP (Arm A) or tafasitamab and lenalidomide in addition to six cycles of R-CHOP (Arm B) and will be administered for up to six 21-day cycles.

### 3.1 Definition of Treatment Cycle

A complete treatment cycle is defined as 21 calendar days during which tafasitamab in addition to R-CHOP (Arm A) or tafasitamab and lenalidomide in addition to R-CHOP (Arm B) will be administered according to the following plan.

### Arm A: Tafasitamab in Addition to R-CHOP

Study treatment consisting of tafasitamab and R-CHOP will be in 21-day cycles for 6 cycles as shown in Table 2.

TABLE 2

| | | Dosing days |
|---|---|---|
| Drug | Dose | (21-day cycle) |
| Tafasitamab | 12 mg/kg IV | 1, 8, 15 |
| Rituximab | 375 mg/m² IV | 1 |
| Cyclophosphamide | 750 mg/m² IV | 1 |
| Doxorubicin | 50 mg/m² IV | 1 |
| Vincristine | 1.4 mg/m² (max 2.0 mg total) IV | 1 |
| Prednisone/Prednisolone | 100 mg/day p.o. | 1-5 |

IV= intravenous,
p.o.= per os

### Arm B: Tafasitamab Plus Lenalidomide in Addition to R-CHOP

Study treatment consisting of tafasitamab plus lenalidomide in addition to R-CHOP will be administered in 21-day cycles for 6 cycles as shown in Table 3.

TABLE 3

| | | Dosing days |
|---|---|---|
| Drug | Dose | (21-day cycle) |
| Tafasitamab | 12 mg/kg IV | 1, 8, 15 |
| Lenalidomide* | 25 mg/day p.o. | 1-10 |
| Rituximab | 375 mg/m² IV | 1 |
| Cyclophosphamide | 750 mg/m² IV | 1 |
| Doxorubicin | 50 mg/m² IV | 1 |
| Vincristine | 1.4 mg/m² (max 2.0 mg total) IV | 1 |
| Prednisone/Prednisolone | 100 mg/day p.o. | 1-5 |

IV= intravenous,
p.o.= per os
*Lenalidomide: Patients will self-administer a starting dose of 25 mg oral lenalidomide daily on Days 1-10 of each 21-day cycle. Dose modification due to toxicity is allowed in 5 mg steps. The minimum dose of lenalidomide is 10 mg on Days 1-10. Please refer to section 3.5 for the lenalidomide dose reduction guidelines.

### 3.2 Investigational Medicinal Product(s)

### Tafasitamab

Tafasitamab Dosage form, Packaging, Storage and Preparation

Tafasitamab drug product (DP) is a yellowish lyophilisate supplied in single-use 20 mL glass vials. Each vial contains 200 mg of tafasitamab for reconstitution with 5 mL water for injection (WFI). Reconstitution yields 40 mg/mL tafasitamab in 25 mM sodium citrate, 200 mM trehalose and 0.02% (w/v) polysorbate 20 at pH 6.0. Each product vial is intended to deliver 200 mg of tafasitamab in 5 ml of reconstituted solution. The solution after reconstitution is colorless to slightly yellow and essentially free of foreign particles; it may contain a few white to whitish product-related particles.

For administration, tafasitamab will be diluted into a commercially available 250 mL infusion container with 0.9% (w/v) sodium chloride for injection.

The individual tafasitamab infusion will be prepared under aseptic conditions and administered at the study site. In general, a vial of tafasitamab is used as soon as possible after reconstitution with WFI. After dilution for infusion, administration of tafasitamab should take place as soon as possible.

### Tafasitamab Administration

Tafasitamab will be administered IV at a dose of 12 mg/kg body weight for 6 cycles. Each 21-day cycle (cycles 1-6) will comprise of tafasitamab infusions on Day 1, Day 8 and Day 15, i.e. each patient will be treated with a maximum of 18 infusions of tafasitamab over the 6 cycles.

For the first infusion, the IV infusion rate should be 70 mL/h for the first 30 minutes and subsequently increased to a rate of 125 mL/h; the total infusion duration will be approximately 2.5 hours.

All subsequent tafasitamab infusions will be administered IV at a constant rate of approximately 125 mL/h over an approximately 2-hour period.

The infusion rate escalation schedules in this protocol are recommendations. If needed, the investigator should use clinical judgement to optimize patient safety by administering the infusion more slowly.

### Lenalidomide

### Lenalidomide Administration

Patients will self-administer a starting dose of 25 mg oral lenalidomide daily on Days 1-10 of each 21-day cycle. Lenalidomide dose may be reduced according to the guidelines described in 3.4.

### R-CHOP

### R-CHOP Administration

Rituximab is advised to be given approximately 30 minutes after the tafasitamab infusion, followed by the CHOP chemotherapy which will be given approximately 30 minutes after the end of the rituximab infusion.
Note: The Day 1 steroid dose being part of CHOP (100 mg prednisone or prednisolone or equivalent, IV or PO) can be used as further component of premedication prior to Tafasitamab infusion.

### 3.3 Treatment Compliance and Product Accountability

The dosing of tafasitamab will be considered appropriate if the tafasitamab dose administered is ≥80% to ≤120% of the assigned dosage per single infusion.

Lenalidomide is to be dispensed at the initiation of each new treatment cycle for treatment from D1-10. A patient will be considered compliant with the protocol if the planned lenalidomide dose administered is ≥80% to 100% of the assigned dosage.

### 3.4 Recommended Dose Modifications, Drug Interruptions and Discontinuation Guidelines

Lenalidomide may be given only on Day 1 to 10 of each cycle and must not be administered beyond this period.

The dose of lenalidomide may be reduced successively level by level from the starting dose of 25mg daily. This is described in below Table 4.

TABLE 4
Lenalidomide Dose Modification Guidelines

| | |
|---|---|
| Starting dose | 25 mg daily on Days 1-10 of |
| | each 21-day cycle |
| Dose Level −1 | 20 mg daily on Days 1-10 of |
| | each 21-day cycle |
| Dose Level −2 | 15 mg daily on Days 1-10 of |
| | each 21-day cycle |
| Dose Level −3 | 10 mg daily on Days 1-10 of |
| | each 21-day cycle |

Lenalidomide may be interrupted (up to 3 days) within the 10-day dosing period and may be restarted within this period at the same dose or at dose level −1, but may not be extended beyond day 10 of this cycle. If lenalidomide dosing was interrupted during the previous cycle and was restarted with a one-level dose reduction without requiring an interruption for the remainder of the cycle, then that reduced dose level will be initiated on Day 1 of the next cycle. There will be no more than one dose reduction from one cycle to the next. Once a patient's lenalidomide dose has been reduced, no dose re-escalation is permitted.

Patients who cannot tolerate Dose Level -3 are to be discontinued from lenalidomide treatment in arm B but should continue therapy with tafasitamab plus R-CHOP for the total duration of six cycles, if possible.

### Criteria to Start Next Treatment Cycle (day 1 of Cycle 2-6)

The next cycle of treatment may begin on the scheduled Day 1 if the following criteria are met:
Absolute Neutrophil Count (ANC) 1,000/mm3 (unless neutropenia is due to infiltration of bone marrow)
Platelets ≥75 000/mm3 (unless thrombocytopenia is due to infiltration of bone marrow)
All other toxicities have resolved to S Grade 2

In case of overlapping toxicities it should be ensured that study drugs (lenalidomide, tafasitamab) are reduced or interrupted or discontinued before any dose reductions of R-CHOP.

If the above mentioned criteria are not met on Day 1 of the planned new cycle, the next cycle should not be commenced. The patient will be evaluated again within 7 days. If the above mentioned criteria are met at any time within 7 days, the next treatment cycle may be initiated.

If the above mentioned criteria are still not met after 7 days delay, the next cycle should not commence. The patient will be evaluated again after another 7 days (or earlier). If the above mentioned criteria are met at any time within 7 days, the next treatment cycle may be initiated.

For Patients in Arm B, lenalidomide should be decreased to the next lower dose level. If lenalidomide was already at the lowest dose level, lenalidomide treatment should be permanently discontinued.

If the above mentioned criteria are still not met after 14 days delay, the next cycle should not commence. The complete blood count should be repeated at a frequency deemed appropriate by the investigator. If the above mentioned criteria are met in Arm B, lenalidomide should be permanently discontinued and tafasitamab should be interrupted in this treatment cycle while R-CHOP treatment may be resumed. In Arm A tafasitamab should be interrupted, while R-CHOP treatment may be resumed.

### 3.5 Concomitant Medication

### Pre-Medication for Tafasitamab Infusions

Tafasitamab infusions should be administered to patients after pre-medication with oral acetaminophen (e.g., 650-1000 mg), an antihistamine such as diphenhydramine hydrochloride (50-100 mg) and glucocorticosteroids (e.g. 100 mg IV prednisone or prednisolone or equivalent) 30-60 minutes prior to starting each infusion (unless contraindicated). Note: the Day 1 steroid dose being part of CHOP (100 mg prednisone or prednisolone or equivalent, IV or PO) can be used as further component of premedication prior to Tafasitamab infusion. Premedication is needed for the first cycle. For patients who do not experience ≥Grade 2 IRRs/≥Grade 1 CRSs to tafasitamab during the first cycle, premedication will be optional for subsequent antibody infusions at the discretion of the investigator. Otherwise, the premedication should be continued for subsequent administrations.

### Steroid Pre-Phase

In patients with urgent need for a steroid pre-phase before initiation of therapy, the use of oral prednisone 25-100 mg/d or equivalent over 7 days is allowed after screening tumor investigations (imaging, blood samples) have been completed.

In exceptional circumstances and at the discretion of the investigator, the steroid pre-phase can be started prior to acquisition of PET.

### 4. Clinical Trial Procedures

### 4.1 Demographic Data/Relevant Medical History and Current Medical Conditions/Baseline Stage and Prognostic Classification

Demographic variables to be recorded will include age, gender, race/ethnic origin. At the time of signing of the ICF, relevant medical history and current medical conditions should be recorded. The medical history of DLBCL should be documented in detail, including all symptoms at screening. Also, examinations leading to the diagnosis of DLBCL should be documented in the patient's source documents. This may include, for example, results of laboratory examinations, imaging results, or clinical symptoms related to DLBCL. The assessment of the lymphoma should include disease staging. In order to reflect the patient's status at the time of screening, the standard Ann Arbor staging system used for DLBCL reflecting the number of sites of involvement and their relation to the diaphragm, the existence of B-symptoms, and the presence of extranodal disease, will be documented (Appendix B). Additionally, the disease risk assessment as per IPI ((Appendix C) and patient status as per Eastern Cooperative Oncology Group (ECOG) performance status criteria (see Appendix A), will be recorded.

Screening for CNS lymphoma involvement is not mandatory. Lumbar puncture with cerebrospinal fluid evaluation (cytology, flow cytometry) and/or head CT/head MRI is recommended in patients with high risk disease to exclude CNS lymphoma involvement.

### 4.2 Bone Marrow Assessment

As per the Lugano criteria (Cheson, 2014), a bone marrow aspiration and biopsy is not mandated in patients who undergo PET/CT or PET/MRI. However, bone marrow assessments may be performed according to investigator's discretion. The data of such examination will be collected in the eCRF.

### 4.3 Radiographic Imaging Assessment

A CT scan (with contrast unless contraindicated) covering at least the neck, chest, abdomen, pelvis, and any other disease sites as well as PET scans are required for the pre-treatment tumor assessment. The use of historical PET/CT or PET/MRI scans within a maximum of 21 days before signature of ICF is permitted as long as they are of acceptable quality and cover the aforementioned anatomical areas. Information on extranodal involvement (e.g. gastric or skin involvement) will be recorded in the source documents.

During the course of the study, response assessments will be performed covering the aforementioned anatomical areas as for screening unless additional regions, are deemed required to be covered.

A mid-treatment CT/MRI should be performed at Cycle 3 D18+/−3 days, i.e. prior to the end of cycle 3; a mid-treatment PET/CT (or PET/MRI) is optional and should be triggered by local guidelines.

An end of treatment PET/CT or PET/MRI should be performed 4-8 weeks after the last study treatment.

During the follow-up period CT scans should be performed roughly every 6 months.

If disease progression/relapse is diagnosed purely on the basis of clinical symptoms, a CT scan with IV contrast (or MRI if IV contrast is contraindicated) or PET/CT (or PET/MRI) is required within 4 weeks of diagnosing disease progression/relapse based on symptoms. If such imaging was performed, it does not need to be repeated at the Early Treatment Discontinuation visit.

NOTE: PET/CT hybrid scanners may be used to acquire the required CT images only if the CT produced by the scanner is of diagnostic quality.

If using a hybrid machine to acquire both PET and CT, the PET should be performed prior to the CT with IV contrast as to not compromise PET results. If independent CT and PET scanners are used, and the patient is receiving both scans on the same day, the PET should be performed prior to the CT with IV contrast. Assessment of PET results is based on Lugano classification (Cheson, 2014; see (Appendix D)

Lesion measurements and other parameters relevant for the response assessment based on Lugano classification (Cheson, 2014; see Appendix D) will be collected in the eCRF.

### 5.0 Efficacy, Pharmacokinetic, Safety and Other Variables

### 5.1 Efficacy Assessments

Efficacy assessments will be made according to the revised response criteria for malignant lymphoma based on the guidelines of the Lugano Classification (as reported by Cheson, 2014) and will be based on investigator assessment (Appendix D).

Efficacy will be evaluated in terms of ORR, DoR, PFS, EFS, OS, TTP and TTNT (Please see section 6.9 and section 6.10 for the definition of efficacy endpoints). Imaging assessment of efficacy/disease response will be recorded at the end of cycle 3 and after the end of treatment (6±2 weeks after day 21 of the last treatment cycle the patient started) as well as approximately every 6 months during the FU period.

### 5.2 Safety Assessments

### Local Safety and Hematology Laboratory Testing

Any abnormal laboratory findings that constitute an AE should be reported as such and should be followed up until the outcome is known. Also, additional diagnostic tests may be indicated to determine a more precise diagnosis of the patient's condition (e.g., ordering a white blood cell (WBC) differential count to help characterise a high or low WBC count, or ordering a determination of red blood cell (RBC) indices to help characterise a low hematocrit).

### Definition of Adverse Events, Serious Adverse Events and Adverse Events of Special Interest

An AE is defined as any untoward medical occurrence in a patient administered a medicinal product, which does not necessarily have a causal relationship to this treatment.

An AE can therefore be any unfavorable and unintended sign (including an abnormal laboratory finding), symptom, or disease temporally associated with the use of a study drug, whether or not it is considered related to that study drug.

AEs include any clinically significant deterioration of a patient's medical status after the signing of the ICF. Also, an increase in the frequency or intensity of a pre-existing event or conditions and events resulting from protocol mandated procedures (e.g., invasive procedures) fall under the definition of AEs.

### Evaluation of AEs to Determine the Following:

Relationship to the study drug or R-CHOP (suspected/not suspected)

Duration (start and end date, or if continuing at end of study)

Intensity: the intensity of all AEs will be graded as mild, moderate, or severe using the following definitions:
mild: tolerable
moderate: interferes with normal activity
severe: incapacitating (causes inability to perform usual activities or work)

Severity, i.e., toxicity grade: determined according to the NCI-CTCAE version 5.0, using the following definitions:
grade 1: mild; asymptomatic or mild symptoms; clinical or diagnostic observations only; intervention not indicated
grade 2: moderate; minimal, local or noninvasive intervention indicated; limiting age-appropriate instrumental activities of daily living (refers to preparing meals, shopping for groceries or clothes, using the telephone, managing money, etc.)
grade 3: severe or medically significant but not immediately life-threatening; hospitalization or prolongation of hospitalization indicated; disabling; limiting self-care activities of daily living
grade 4: life-threatening consequences; urgent intervention indicated
grade 5: death related to AE

Outcome
All non-serious AEs must be followed up for a final outcome. An outcome of “unknown” is not considered to be an acceptable final outcome. An outcome of “not yet resolved” is an acceptable final outcome for non-serious AEs at the end of a patient's participation in the study. All SAES must be followed up for a finaloutcome until resolution or, if resolution becomes unlikely, until stabilization or death.

Action taken (no action taken; study drug or R-CHOP temporarily interrupted; study drug or R-CHOP permanently discontinued due to this AE; medication taken; non-drug therapy given; hospitalization/prolonged hospitalization)

Seriousness: an SAE is defined as serious if it:
results in death
is life-threatening
requires inpatient hospitalization or prolongation of existing hospitalization (hospitalization signifies that the patient was an inpatient for at least one overnight stay) unless hospitalization is for:
   routine treatment or monitoring of the studied indication, not associated with deterioration of symptoms related to DLBCL
   elective or preplanned treatment for a pre-existing condition that is unrelated to DLBCL and has not worsened since signing of the informed consent social reason and respite care in the absence of any deterioration in the patient's general condition
results in persistent or significant disability or incapacity
is a congenital anomaly or birth defect
is medically significant, i.e., defined as an event that jeopardizes the patient or may require medical intervention to prevent one of the outcomes listed previously.

The term “life-threatening” refers to an event in which the patient was, in the view of the reporting Investigator, at immediate risk of death at the time of the event; it does not refer to an event that hypothetically might have caused death if it were more severe. Medical judgment should be exercised in deciding whether an AE is serious in other situations: important AEs that are not immediately life-threatening or do not result in death or hospitalization but may jeopardize the patient or may require intervention to prevent one of the other outcomes listed in the previous definitions should also be considered as serious.

AEs of special interest (AESIs) for tafasitamab are: TLS, IRRs and allergic reactions to study drug ≥grade 3, cytokine release syndrome, second primary malignancies, hepatitis B reactivation, progressive multifocal leukoencephalopathy (PML).

AEs of special interest (AESIs) for lenalidomide: Second primary malignancies.

Unlike routine safety assessments, SAEs and AESIs are monitored continuously and have special reporting requirements.

### 6. Statistical Methods and Planned Analysis

### 6.1 General Statistical Considerations

Tabulations of summary statistics, graphical presentations, and statistical analyses will be performed using SAS® software version 9.3 or higher.

Continuous, quantitative variable summaries will include the number of patients (N) (with non-missing values/valid cases), mean, standard deviation, minimum, 25th quartile, median, 75th quartile and maximum, except for PK metrics, where additional statistics may be used.

Categorical, qualitative variable summaries will include the frequency and percentage of patients/entries in the particular category.

Definition of baseline value: the last pre-administration observation will be used as the baseline value for calculating post-administration changes from baseline. All data obtained via the eCRF and entered into the database will be provided in separate data listings showing individual patient's values. A Statistical Analysis Plan (SAP) detailing the statistical analyses will be finalized prior to first patient first visit.

The planning and reporting of statistical analysis will be carried out as described in the sponsor's SOPs governing clinical trials.

The sponsor and/or designated CRO will analyze the data. Any data analysis carried out independently by the investigator should be submitted to the sponsor before publication or presentation.

It is planned that the data from participating centers in this protocol will be combined, so that an adequate number of patients will be available for analysis.

### 6.2 Timing of Analysis

### Safety Run-in Analysis

Patient profiles and Listings of Adverse events will be provided in case a Safety Data Review is performed.

### Primary Completion Analysis

The primary completion analysis will be performed based on data cut-off 30 days after all patients have performed their End of Treatment Visit (EOT). Primary and Key Secondary Objectives will be analysed at the time of primary completion. Details will be provided in the SAP.

### Final Analysis

After the last patient completed the last visit, a final analysis will be performed. At the time of Final Analysis, analyses performed during Primary Completion Analysis will be repeated using updated data in addition to the performance of Secondary and exploratory objectives.

### 6.3 Population for Analysis

Patients who were screened but never started study treatment will be listed. Screening failures will not be included in any of the summary tables (except of the patient disposition table).

### Full Analysis Set (FAS)

All patients who are randomized to either study arm will be included in FAS. Efficacy analysis will be performed on FAS. Patients will be analyzed according to the study treatment they were randomized to.

### Safety Set (SAF)

All patients who received at least one dose of study drug (tafasitamab or tafasitamab plus lenalidomide). Safety analyses will be performed on SAF.

Patients will be analyzed according to the study treatment they actually received, which is defined as the treatment the patient received on the first day of study treatment.

### Per Protocol Set (PPS)

Patients included in FAS without any important protocol deviation that would influence efficacy endpoints.

All protocol deviations or conditions leading to exclusion from the PPS will be detailed in the data handling plan and statistical analysis plan. Sensitivity analyses for efficacy endpoints may be performed using PPS.

### 6.4 Patient Disposition, Demographics and Baseline Characteristics

A table will be provided with the following information:

Number of patients included in each analysis set.

Number of patients screened, randomized, received at least one dose of trial treatment, discontinued treatment within first 21 days, discontinued treatment during the 6 cycles of treatment, prematurely discontinued trial and finished complete follow-up and had their scheduled last-visit. Reasons of end of treatment and end of study will be provided.

Number of patients withdrawn from the trial and the reason for withdrawal.

Demographic information will be summarised using descriptive statistics for the FAS. Gender and race/ethnic origin will be summarised by counts and percentages.

Medical history will be summarised by counts and percentages using MedDRA system organ class (SOC) and preferred term classifications. Concomitant medications will be recorded and coded using the WHO Drug Dictionary Enhanced and grouped by Anatomical Therapeutic Chemical (ATC) classes. Tabulations with counts/percentages will show the number of medications/percentage used in each class.

The following baseline characteristics and medical history related to DLBCL will be summarised displaying the

duration of disease since initial diagnosis

IPI

Ann Arbor staging

Bulky vs non-bulky disease

COO (from local lab, if available, and which assay used e.g. gene expression profiling or IHC)

B symptoms

Extranodal involvement yes or no

Number of sites of extranodal involvement

Bone marrow involvement by PET yes or no

Bone marrow involvement by biopsy yes, no, not available

LDH above upper limit of normal yes or no

Details will be provided in the Statistical Analysis Plan.

### 6.5 Treatments (Study Treatment, Concomitant Therapies, Compliance)

### Study Treatment

Duration of study treatment exposure and cumulative dose will be summarized by treatment arm. The number of patients with dose changes/interruptions will be presented by treatment arm, along with reasons for the dose change/interruption. The safety set will be used for the tables and listings.

### Prior and Concomitant Therapies

Corticosteroids administered/taken within three weeks before ICF signature and concomitant medications and significant non-drug therapies taken concurrently with the study treatment will be listed and summarized by Anatomical Therapeutic Chemical Classification System (ATC) term, preferred term and treatment arm. These summaries will include medications starting on or after the start of study treatment or medications starting prior to the start of study treatment and continuing after the start of study treatment.

The safety set will be used for all above mentioned concomitant medication tables and listings.

### 6.6 Sample Size Determination

As this is a Phase lb study primarily conducted to explore safety endpoints, no formal statistical hypothesis has been established for the sample size calculation of this trial.

With a sample size of 12 patients in each arm, there is a 60% probability to observe 4 or more patients with unacceptable toxicity if the underlying incidence rate of these toxicities is 33%.

With a sample size of 30 patients in each arm, there is a 55% probability to observe 10 or more patients with unacceptable toxicity if the underlying incidence rate of these toxicities is 33%.

### 6.7 Primary Objective Analysis

The Primary objective of this trial is to assess the safety and tolerability of tafasitamab in addition to R-CHOP and tafasitamab plus lenalidomide in addition to R-CHOP in patients with newly diagnosed DLBCL. To assess safety and tolerability, the incidence and severity of hematological and non-hematological AEs including clinically significant laboratory abnormalities will be determined. AEs will be categorized with regards to seriousness, intensity, toxicity, study treatment relationship, outcome and action taken. AE reports will be graded according to National Cancer Institute (NCI) Common Terminology Criteria for adverse events (CTCAE), version 5.0.

### 6.8 Key Secondary Objective Analysis

To assess efficacy of tafasitamab in addition to R-CHOP and tafasitamab plus lenalidomide in addition to R-CHOP in terms of ORR and PET-negative CR rate at end of treatment.

### Objective Response Rate at the End of Treatment

The ORR is defined as the proportion of patients with CR or PR based on the response achieved at the end of treatment (tumor scans performed until 56 days after last date of study drug administration).

The ORR along with 95% exact CI (using Clopper-Pearson exact method) will be presented for both treatment arms.

The number and percentage of patients with CR and the number of patients with PR will be presented by treatment arm.

### The Metabolic PET-Negative Complete Response Rate at the End of Treatment

The metabolic PET-negative CR rate is defined as the proportion of patients who achieved metabolic PET-negative CR based on PET/CTs performed 6±2 weeks after End of Treatment.

The metabolic PET-negative CR rate along with 95% exact CI (using Clopper-Pearson exact method) will be presented for both treatment arms.

### 6.9 Secondary Objectives Analysis

Further details on the subsequent analyses will be specified in SAP.

### Long-Term Safety Analysis

Incidence and severity of AEs will be presented for patients in the follow-up period, starting from 31^{st} day after End of Treatment to the End of Study Visit.

### Efficacy Endpoints

To assess efficacy (based on Lugano 2014 criteria) of tafasitamab in addition to R-CHOP and tafasitamab plus lenalidomide in addition to R-CHOP with respect to the following endpoints:

### Best ORR Until End of Study

The best ORR is defined as the proportion of patients with CR or PR based on the best response achieved until the end of study.

The best ORR along with 95% exact CI (using Clopper-Pearson exact method) will be presented for both treatment arms.

The number and percentage of patients with CR and the number of patients with PR will be presented by treatment arm.

### Metabolic PET-Negative Complete Response Rate at the End of Study

The metabolic PET-negative CR rate is defined as the proportion of patients who achieved metabolic PET-negative CR based on PET/CTs performed until end of study.

The metabolic PET-negative CR rate along with 95% exact CI (using Clopper-Pearson exact method) will be presented for both treatment arms.

### Progression Free Survival (PFS) Rate at 12 Months and 24 Months

Tumor assessments will be performed by local radiologists using Lugano 2014 criteria (Cheson, 2014).

PFS is defined as the time from the date of randomization to the date of the first radiologically or histologically/cytologically documented disease progression or death due to any cause. If a patient has not progressed or died at the analysis cut-off date or when he/she receives further anti-neoplastic therapy, PFS will be censored on the date of the last adequate tumor evaluation before the earlier of the cut-off date or start of the further antineoplastic therapy date.

Kaplan Meier plots will be used to estimate the distribution of PFS. The PFS probabilities at 12 and 24 months, and the associate 95% CI will be summarized for each treatment arm.

### Event-Free Survival (EFS) Rate at 12 Months and 24 Months

EFS is defined as the time from the date of randomization to the date of the first radiologically documented disease progression or death due to any cause or start of new anti-lymphoma treatment. If a patient has not progressed or died or started a new anti-lymphoma treatment at the analysis cut-off date, EFS will be censored on the date of last contact.

Kaplan Meier plots will be used to estimate the distribution of EFS. The EFS probabilities at 12 and 24 months, and the associate 95% Cl will be summarized for each treatment arm.

### Time to Next Anti-Lymphoma Treatment (TTNT)

Time to next anti-lymphoma treatment (TTNT) is defined as the time from the date of randomization to the date of administration of next anti-lymphoma treatment or death due to any cause. If a patient has not received next anti-lymphoma treatment or did not die until the analysis cut-off date, he/she will be censored on the date of last contact.

Kaplan Meier plots will be used to estimate the distribution of TTNT. The TTNT probabilities at 12 and 24 months, and the associate 95% CI will be summarized for each treatment arm.

### Overall Survival at 12 Months and 24 Months

Overall survival (OS) is defined as the time from randomization until death from any cause and documented by the date of death.

Kaplan Meier plots will be used to estimate the distribution of OS. The OS probabilities at 12 and 24 months, and the associate 95% CI will be summarized for each treatment arm.

### 6.10 Exploratory Objective Analysis

The following efficacy endpoints:
a. ORR and
b. PFS
   will be assessed in both treatment arms based on the following biomarkers:
i. Cell of origin
ii. NK-cell count in the tumor tissue
iii. NK-cell gene expression signature in the tumor tissue
iv. Macrophage count in the tumor tissue
v. Macrophage gene expression signature in the tumor tissue
vi. Quantitative and semi-quantitative CD19 expression on tumor cells (in diagnostic biopsies and at progression/relapse)
vii. Quantitative and semi-quantitative CD20 expression on tumor cells (in diagnostic biopsies and at progression/relapse)

### 6.11 Safety Analysis

The primary and one of the secondary objective of this study is to assess the safety and tolerability of tafasitamab in addition to R-CHOP and tafasitamab plus Lenalidomide in addition to R-CHOP in adult patients with newly diagnosed DLBCL.

All Safety Analysis will be presented by treatment arms and overall.

### Primary Endpoint:

Incidence and severity of TEAEs.

Treatment emergent adverse events are all adverse events which start after the first dose of study treatment until 30 days after day 21 of the last treatment cycle the patient started.

### Secondary Endpoint:

Incidence and severity of AEs will be presented for patients in the study starting from 31^{st} day after End of Treatment to the End of Study Visit.

Note: At the time of primary completion analysis, all non-treatment emergent AEs collected (AEs prior to first dose of study drug administration and after 30 days of End of Treatment) will be listed.

### Adverse Events

All adverse events which start after the first dose of study treatment until 30 days after day 21 of the last treatment cycle the patient started will be considered as a treatment emergent adverse event (TEAE). Adverse events that start during the study but before the time of the first dose of study treatment (e.g. screening period) will be classified as a non-treatment emergent adverse event and will be included in adverse events listings, but will not be summarized.

TEAEs will be coded according to MedDRA SOC and preferred terms. Incidence and frequency of all AEs will be summarised by SOC, preferred term, relationship to treatment, severity and seriousness.

An AE summary table will be presented showing the number of events, number of subjects and the percentage of subjects in each arm and overall having:

All Treatment-emergent adverse events (TEAEs)

TEAEs by maximum severity

SAEs

Drug-related TEAEs

Drug-related TEAEs in each severity/toxicity grading

TEAEs that led to treatment discontinuation

IRRs by grade.

Adverse Events of Special Interests in this study are:

Infusion-related reactions ≥grade 3

Cytokine release syndrome

Allergic reactions to tafasitamab ≥grade 3

Second primary malignancies

PML

Hepatitis B reactivation

TLS

The sponsor will describe AESIs, in addition to those reported as SAEs. AESI tabulations will be analogous to the tabulation of TEAEs.

The sponsor will discuss other significant AEs as appropriate, e.g. laboratory abnormalities that qualify as AEs (other than those meeting the definition for serious) and any events that led to an intervention (including premature discontinuation of IMP, increase of dose interval, or significant additional concomitant therapy), in addition to those reported as SAEs.

In addition to the investigator's evaluation of normal or abnormal, the sponsor will internally evaluate each clinical laboratory result, vital sign result, and ECG result for whether it reflects a new abnormality, and for numeric data, whether it reflects a significant worsening from baseline or an outlying result or extreme value. These terms are defined for clinical laboratory results, vital sign results, and ECG results as follows:

A new abnormality will be any abnormal post baseline result for a patient whose baseline was within normal limits.

A significant worsening will be any numeric clinical laboratory result, vital sign result, or ECG interval measurement that represents a change from baseline by greater than or equal to 25% of the baseline value, in the direction away from normal (i.e., in the direction that is clinically significant).

An outlying result for any numeric laboratory result, vital sign result, or ECG interval measurement will be any post-administration change from baseline that meets either of the following criteria:
<25th Percentile−1.5*(interquartile range) OR
>75th Percentile+1.5*(interquartile range).

An extreme value for any numeric laboratory result, vital sign result, or ECG interval measurement will be any post-administration change from baseline that meets either of the following criteria:
<25th Percentile−3*(interquartile range) OR
>75th Percentile+3*(interquartile range).

### 7. Preliminary Data

Eighty-two patients were screened and 66 underwent randomization; 33 were allocated to Arm A and 33 were allocated to Arm B. Table 5 shows the baseline characteristics.

TABLE 5

| | | Arm A: | Arm B: | |
|---|---|---|---|---|
| | | tafasitamab | tafasitamab + | |
| | | + | lenalidomide + | |
| | | R-CHOP | R-CHOP | Total |
| | | (n = 33) | (n = 33) | (N = 66) |
| Age at screening | Median | 66.0 | 64.0 | 64.5 |
| (years) | Min, Max | 43, 86 | 20, 79 | 20, 86 |
| Age categories at | <60 | 12 (36.4) | 11 (33.3) | 23 (34.8) |
| screening (years), | ≤60 | 21 (63.6) | 22 (66.7) | 43 (65.2) |
| n (%) | | | | |
| Sex, n (%) | Male | 15 (45.5) | 13 (39.4) | 28 (42.4) |
| | Female | 18 (54.5) | 20 (60.6) | 38 (57.6) |
| Pre-planned | Yes | 4 (12.1) | 4 (12.1) | 8 (12.1) |
| radiotherapy | No | 29 (87.9) | 29 (87.9) | 58 (87.9) |
| at screening, n (%) | | | | |
| Pre-planned CNS | Yes | 6 (18.2) | 8 (24.2) | 14 (21.2) |
| prophylaxis with IV | No | 27 (81.8) | 25 (75.8) | 52 (78.8) |
| methotrexate, n (%) | | | | |
| Pre-planned CNS | Yes | 7 (21.2) | 3 ( 9.1) | 10 (15.2) |
| prophylaxis with | No | 26 (78.8) | 30 (90.9) | 56 (84.8) |
| intrathecal CT, n (%) | | | | |
| Ann Arbor disease | Stage I | 2 ( 6.1) | 1 ( 3.0) | 3 ( 4.5) |
| stage, n (%) | Stage II | 0 | 1 ( 3.0) | 1 ( 1.5) |
| | Stage III | 6 (18.2) | 7 (21.2) | 13 (19.7) |
| | Stage IV | 24 (72.7) | 24 (72.7) | 48 (72.7) |
| | Missing | 1 ( 3.0) | 0 | 1 ( 1.5) |
| IPI risk score, n (%) | IPI 2 | 10 (30.3) | 9 (27.3) | 19 (28.8) |
| | IPI 3 | 14 (42.4) | 15 (45.5) | 29 (43.9) |
| | IPI 4 | 8 (24.2) | 9 (27.3) | 17 (25.8) |
| | IPI 5 | 0 | 0 | 0 |
| | Missing | 1 ( 3.0) | 0 | 1 ( 1.5) |
| Bulky disease >10 | Present | 15 (45.5) | 15 (45.5) | 30 (45.6) |
| cm, n (%) | Absent | 17 (51.5) | 18 (54.5) | 35 (53.0) |
| | Missing | 1 ( 3.0) | 0 | 1 ( 1.5) |
| ECOG at baseline, | ECOG 0 | 20 (60.6) | 10 (30.3) | 30 (45.5) |
| n (%) | ECOG 1 | 10 (30.3) | 20 (60.6) | 30 (45.5) |
| | ECOG 2 | 3 ( 9.1) | 3 ( 9.1) | 6 ( 9.1) |

At the data cut-off, two patients in Arm A had discontinued treatment due to adverse events, while there were no discontinuations in Arm B. The following

Table 6 shows the status of the patients in the trial at data cut-off.

TABLE 6

| | | Arm B: | |
|---|---|---|---|
| | Arm A: | tafasitamab + | |
| | tafasitamab + | lenalidomide + | Total |
| | R-CHOP | R-CHOP | (N = 66), |
| Treatment cycles | (n = 33), n (%) | (n = 33), n (%) | n (%) |
| Total number of patients | | | |
| entered into: | | | |
| Cycle 1 | 32* (97.0) | 33 (100) | 65 (98.5) |
| Cycle 2 | 29 (87.9) | 30 (90.9) | 59 (89.4) |
| Cycle 3 | 28 (84.8) | 30 (90.9) | 58 (87.9) |
| Cycle 4 | 23 (69.7) | 23 (69.7) | 46 (69.7) |
| Cycle 5 | 16 (48.5) | 21 (63.6) | 37 (56.1) |
| Cycle 6 | 13 (39.4) | 14 (42.4) | 27 (40.9) |

Overall, 98.5% of patients experienced TEAEs; of these 75.8% were grade 3 or higher. Serious TEAEs were experienced by 29 total patients (43.9%), 13 patients in Arm A and 16 patients in Arm B (39.4% vs 48.5%). No new safety signals were identified with either tafasitamab plus R-CHOP or tafasitamab plus lenalidomide plus R-CHOP compared with previous Phase III studies with R-CHOP, lenalidomide or R2-CHOP. The following Table 7 provides a summary of TEAEs.

TABLE 7

| | Arm A: | Arm B: | |
|---|---|---|---|
| | tafasitamab + | tafasitamab + | |
| Overall | R-CHOP | lenalidomide + | Total |
| summary by | (n = 33), | R-CHOP | (N = 66), |
| toxicity grade | n (%) [E] | (n = 33), n (%) [E] | n (%) [E] |
| Patients | 32* (97.0) [345] | 33 (100) [443] | 65 (98.5) [788] |
| with TEAEs | | | |
| and the total | | | |
| number of events | | | |
| Grade 1 | 26 (78.8) [140] | 27 (81.8) [161] | 53 (80.3) [301] |
| Grade 2 | 27 (81.8) [120] | 28 (84.8) [135] | 55 (83.3) [255] |
| Grade 3 | 21 (63.6) [48] | 22 (66.7) [72] | 43 (65.2) [120] |
| Grade 4 | 13 (39.4) [36] | 19 (57.6) [75] | 32 (48.5) [111] |
| Grade 5 | 1 (3.0) [1] | 0 | 1 (1.5) [1] |
| Grade 3 or higher | 23 (69.7) [85] | 27 (81.8) [147] | 50 (75.8) [232] |
| | Arm A: | Arm B: | |
| | tafasitamab + | tafasitamab + | |
| | R-CHOP | lenalidomide + | Total |
| Overall summary | (n = 33), | R-CHOP | (N = 66), |
| of serious TEAEs | n (%) [E] | (n = 33), n (%) [E] | n (%) [E] |
| Patients | 13 (39.4) [28] | 16 (48.5) [27] | 29 (43.9) [55] |
| with serious | | | |
| TEAEs and the | | | |
| total number | | | |
| of events | | | |

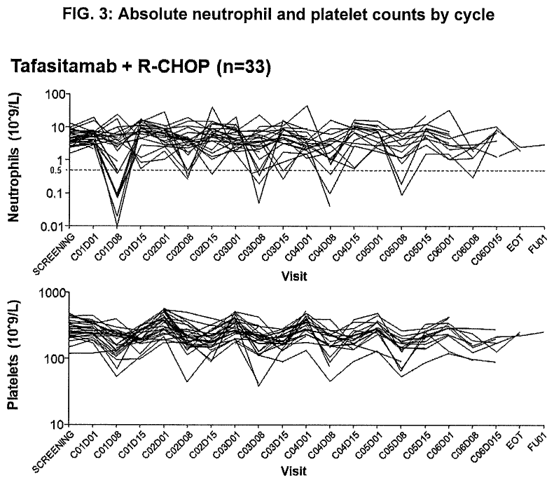

The most frequent events by SOC were blood and lymphatic system disorders, experienced by 25 patients in each arm (75.8%). More blood and lymphatic disorder events occurred in Arm B than Arm A (163 v. 94), with a higher incidence of greater than or equal to 3 events (116 vs 57 in Arm B vs Arm A). FIG. 2 provides a summary.

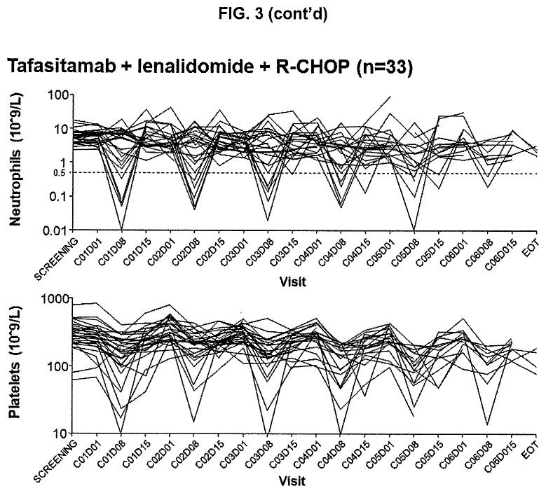

The higher rate of blood and lymphatic system disorder events in Arm B was driven by higher incidence of neutropenia and thrombocytopenia with lenalidomide than without (ten patients (30.3) vs three patients (9.1%) had thrombocytopenia). Of these patients, eight (24.2%) experienced grade greater than or equal to 3 events with lenalidomide compared with two patients without (6.1%). FIG. 3 provides data.

In Arm A, three patients (9.1%) had febrile neutropenia compared with four patients (12.1%) in Arm B. Grade 3 or higher infection events were experienced by six (18.2%) and seven (21.2%) patients in Arm A and Arm B, respectively. One patient died due to a urinary tract infection in Arm A. Table 8 provides information concerning adverse events of interest.

TABLE 8

| | | | Arm B: | |
|---|---|---|---|---|
| | | Arm A: | tafasitamab + | |
| | | tafasitamab | lenalidomide | |
| | | + | + | |
| | | R-CHOP | R-CHOP | Total |
| | | (n = 33), | (n = 33), | (N = 66), |
| | | n (%) [E] | n (%) [E] | n (%) [E] |
| Neutropenia | Any grade | 16 (45.5) [39] | 19 (57.6) [64] | 34 (51.6) [103] |
| | Grade 3 | 14 (42.4) [32] | 19 (67.6) [62] | 33 (50.0) [84] |
| | or higher | | | |
| Anemia | Any grade | 14 (42.4) [27] | 10 (30.3) [22] | 24 (36.4) [49] |
| | Grade 3 | 6 (16.2) [7] | 6 (18.2) [10] | 11 (16.7) [17] |
| | or higher | | | |
| Thrombo- | Any grade | 3 (9.1) [7] | 10 (30.3) [29] | 13 (19.7) [36] |
| cytopenia | Grade 3 | 2 (6.1) [3] | 8 (24.2) [18] | 10 (15.2) [21]. |
| | or higher | | | |
| Pulmonary | Any grade | 1 (3.0) [1] | 1 (3.0) [1] | 2 (3.0) [2] |
| embolism | Grade 3 | 1 (3.0) [1] | 1 (3.0) [1] | 2 (3.0) [2] |
| | or higher | | | |
| Deep vein | Any grade | 1 (3.0) [1] | 1 (3.0) [1] | 2 (3.0) [2] |
| thrombosis | Grade 3 | 1 (3.0) [1] | 1 (3.0) [1] | 2 (3.0) [2] |
| | or higher | | | |
| Febrile | Any grade | 3 (9.1) [3] | 4 (12.1) [5] | 7 (10.6) [8] |
| neutropenia | Grade 3 | 3 ( 9.1) [3] | 4 (12.1) [5] | 7 (10.6) [8] |
| | or higher | | | |
| Diarrhea | Any grade | 7 (21.2) [8] | 9 (27.3) [17] | 16 (24.2) [26] |
| | Grade 3 | 1 (3.0) [1] | 0 | 1 (1.5) [1] |
| | or higher | | | |
| Vomiting | Any grade | 8 (24.2) [10] | 4 (12.1) [6] | 12 (18.2) [16] |
| | Grade 3 | 1 ( 3.0) [1] | 0 | 1 (1.5) [1] |
| | or higher | | | |
| Infections | Any grade | 13 (39.4) [20] | 16 (48.5) [20] | 29 (43.9) [40] |
| | Grade 3 | 6 (18.2) [6] | 7 (21.2) [9] | 13 (19.7) [15] |
| | or higher | 0 | 2 (6.1) [2] | 2 (3.0) [2] |
| | Pneumonia | | | |
| | (any grade) | | | |
| Infusion- | Any grade | 4 (12.1) [4] | 4 (12.1) [6] | 8 (12.1) [10] |
| related | Grade 3 | 0 | 1 (3.0) [1] | 1 (1.5) [1] |
| reaction | or higher | | | |
| (with any | | | | |
| treatment) | | | | |

These preliminary data suggest that R-CHOP can be safely combined with tafasitamab or tafasitamab plus lenalidomide in patients with newly diagnosed treatment naive DLBCL. The incidence of TEAEs was generally comparable between the two treatment arms, with no new safety signals observed to those expected with R-CHOP alone or in combination with lenalidomide (R2-CHOP).

After three treatment cycles, 45 patients (68.2%) were available for interim response assessment. In total, 41/45 patients (91.1%) had an objective response using the 2014 Lugano classification: 19/22 in Arm A and 22/23 in Arm B. In a following response assessment among 60 patients who completed tumor assessments after cycle 3, ORR was 89.7% (arm A) and 93.5% (arm B).

For the 60 patients with a tumor assessment at EoT across both arms combined, the ORR was 86.7% (52/60; 95% confidence interval [CI], 75.4-94.1) and the Complete Response (CR) rate was 66.7% (40/60; 95% CI, 53.3-78.3). For the full analysis set (both arms combined), the ORR and CR rate was 78.8% (95% CI, 67.0-87.9) and 60.6% (95% CI, 47.8-72.4), respectively.

Patients (83) were screened in nine countries across Europe and the US; 66 were randomized (Arm A, n=33; Arm B, n=33). Median age was 64.5 years (range 20-86). Many patients had high-risk disease: IPI 2: 24/66 (36.4%), IPI 3: 29/66 (43.9%), IPI 4: 11/66 (16.7%), IPI 5: 2/66 (3.0%); ECOG PS 2: 6/66 (9.1%). Most patients had Stage III/IV disease 62/66 (93.9%); 29/66 (43.9%) had bulky disease. The average relative dose intensity of R-CHOP in each cycle was maintained in both arms. ORR at EOT was observed in 75.8% patients in Arm A (24 CRs and 1 PR out of 33 patients) and 81.8% patients in Arm B (22 CRs and 5 PRs out of 33 patients).

### Efficacy Data:

ORR at EoT was observed in 25 patients (76%) in Arm A, and in 27 patients (82%) in Arm B.

Best ORR was observed in: Arm A: 90.9% of patients (CR, 29 patients; PR, 1 patient) and in Arm B: 93.9% of patients (CR, 25 patients; PR, 6 patients).

DoR rate at 6 months was 82.6% and 86.2% for patients in Arms A and B, respectively.

DoCR rate at 6 months was 83.6% and 95.2% for patients in Arms A and B, respectively.

### EXAMPLE 2

A Study of Tafasitamab in Combination with Lenalidomide and Rituximab in Subjects with Follicular Lymphoma or Marginal Zone Lymphoma—in MIND

This Phase 3 double-blind, placebo-controlled, randomized study is designed to assess tafasitamab in combination with lenalidomide and rituximab in subjects with relapsed/refractory follicular lymphoma Grade 1 to 3a or relapsed/refractory marginal zone lymphoma.

Participants are included in the study only if all of the following criteria apply:
1. Age greater than or equal to 18 years.
2. Ability to comprehend and willingness to sign a written informed consent form for the study.
3. Histologically confirmed Grade 1, 2, or 3a follicular lymphoma or histologically confirmed nodal marginal zone lymphoma, splenic marginal zone lymphoma, or extranodal follicular lymphoma of the mucosa-associated lymphoid tissue (CD19+ and CD20+ by flow cytometry or immunohistochemistry) as assessed locally.
4. Willingness to avoid pregnancy or fathering children based on the criteria below.
   a. Male participants with reproductive potential must agree to take appropriate precautions to avoid fathering children (with at least 99% certainty) from screening through 180 days (6 months) after the last dose of study treatment, even if they have undergone a successful vasectomy, and must refrain from donating sperm during this period.
   b. Women of childbearing potential participants:
      Must commit either to abstain continuously from heterosexual sexual intercourse or agree to take appropriate precautions to avoid pregnancy (by using 2 different methods of birth control with at least 99% certainty) starting at least 4 weeks before taking the study treatment, while taking the study treatment, during breaks (dose interruptions), and for at least 180 days (6 months) after stopping the study treatment.
      Must have a negative serum pregnancy test at screening (within 10-14 days of the first study drug treatment) and before the first dose on Day 1 (within 24 hours of initiating treatment with lenalidomide).
      Agree to ongoing pregnancy testing during the course of the study; weekly during the first month of study drug treatment, then monthly thereafter for women with regular menstrual cycles or every 2 weeks for women with irregular menstrual cycles (even if true abstinence is the chosen method of birth control) up to and including the end of treatment visit.
      Must refrain from breastfeeding and donating oocytes during the course of study and for 180 days (6 months) after the last dose of study treatment.
   c. A woman not considered to be of childbearing potential is eligible.
5. All participants must:
   a. Have an understanding that lenalidomide could have a potential teratogenic risk.
   b. Abstain from donating blood while taking study treatment and for 28 days after discontinuation of study treatment.
   c. Not share study medication with another person.
   d. Agree to be counseled about pregnancy precautions and risk of fetal exposure.
   e. In the opinion of the investigator, be able and willing to receive adequate mandatory prophylaxis and/or therapy for thromboembolic events (e.g., aspirin 70-325 mg daily or low-molecular-weight heparin).
   f. In the opinion of the investigator, be able to understand and comply with all study-related procedures, medication use, and evaluations.
   g. In the opinion of the investigator, not have a history of noncompliance or be considered potentially unreliable and/or uncooperative.
6. Tumor tissue sufficient for retrospective central pathology review and correlative studies must be provided to participate in this study. A fresh biopsy is preferred if clinically feasible but if not, an archival specimen is acceptable.
7. Must have been previously treated with at least 1 prior systemic anti-CD20 immunotherapy or chemo-immunotherapy. This includes treatments such as the following: rituximab monotherapy or chemotherapy plus immunotherapy with rituximab or obinutuzumab, with or without maintenance. At least 6 doses of anti-CD20 immunotherapy must have been given in prior therapy. Systemic therapy does not include, for example, local involved field radiotherapy for limited stage disease, HBV/HCV therapy, or H pylori eradication.
8. Must have documented relapsed, refractory, or progressive disease after treatment with systemic therapy.
   a. Relapsed lymphoma: relapsed after initial response of complete response to prior therapy.
   b. Refractory lymphoma: achieved less than partial response to the last treatment or achieved a complete response or partial response that lasted less than 6 months before lymphoma progression.
   c. Progressive lymphoma: progressive disease after initial response of partial response or stable disease to prior therapy.
9. Must be in need of treatment for relapsed, refractory, or progressive disease as assessed by the investigator.
   a. Participants must have at least 1 measurable disease site. A radiographically measurable lymphadenopathy is defined as at least 1 nodal lesion >1.5 cm in longest diameter or at least 1 extranodal lesion >1.0 cm in longest diameter. The lesion must be confirmed to be positron emission tomography (PET)-positive at the latest at the time of randomization.
10. Eastern Cooperative Oncology Group (ECOG) performance status of 0 to 2.
11. Participants with laboratory values at screening defined in Table 9.

TABLE 9
Inclusionary Laboratory Values

| | |
|---|---|
| Laboratory Parameter | Inclusion Criterion |

Hematology (hematological laboratory values should be considered
in the absence of growth factors or transfusions)

| | | |
|---|---|---|
| a | Platelets | ≥75 × 10⁹/L (unless secondary |
| | | to bone marrow (BM) |
| | | involvement as demonstrated |
| | | by BM biopsy). |
| b | absolute | ≥1.5 × 10⁹/L (unless secondary |
| | neutrophil | to BM involvement as |
| | count (ANC) | demonstrated by BM biopsy). |
| c | Hemoglobin | ≥0. 8.0 g/dL (unless secondary |
| | | to BM involvement as |
| | | demonstrated by BM biopsy). |

Hepatic

| | | |
|---|---|---|
| d | alanine | ≤3 × upper limit of normal (ULN) |
| | aminotransferase | or <5 × ULN in cases of |
| | (ALT) | documented liver involvement. |
| e | aspartate | ≤3 × ULN or <5 × ULN in |
| | aminotransferase | cases of documented liver |
| | (AST) | involvement. |
| f | Total serum | ≤1.5 × ULN unless secondary |
| | bilirubin | to Gilbert's syndrome or |
| | | documented liver involvement |
| | | by lymphoma. Participants |
| | | with Gilbert's syndrome or |
| | | documented liver involvement by |
| | | lymphoma may be included if their |
| | | total bilirubin is ≤5 × ULN. |
| g | Alkaline | ≤3 × ULN or <5 × ULN in cases of |
| | phosphatase | documented liver involvement. |

Renal

| | | |
|---|---|---|
| h | Serum creatinine | ≥30 mL/minute either measured |
| | clearance | or calculated using a |
| | | standard Cockcroft and Gault formula. |

Participants are Excluded From the Study if Any of the Following Criteria Apply:
1. Women who are pregnant or breastfeeding.
2. Any histology other than follicular lymphoma and marginal zone lymphoma or clinical evidence of transformed lymphoma by investigator assessment.
3. History of radiation therapy to 25% of the BM for other diseases.
4. History of prior non-hematologic malignancy except for the following:
   a. Malignancy treated with curative intent and with no evidence of active disease for more than 2 years before screening.
   b. Adequately treated lentigo maligna melanoma without current evidence of disease or adequately controlled nonmelanomatous skin cancer.
   c. Adequately treated carcinoma in situ without current evidence of disease.
5. Congestive heart failure (left ventricular ejection fraction of <50%, assessed by 2D-echocardiography or multigated acquisition (MUGA) scan.
6. Participants with:
   a. Known positive test result for HCV (with anti-HCV serology testing) and a positive test for HCV RNA. Participants with positive serology must have been tested for HCV RNA and are eligible only in the case of negative HCV RNA.
   b. Known positive test result for chronic HBV infection (defined by HBsAg positivity).
   c. Known seropositive for or history of active viral infection with human immunodeficiency virus.
7. Active systemic infection (including SARS-CoV-2-positive test).
8. Participants in a severely immunocompromised state.
9. Known CNS lymphoma involvement.
10. Uncontrolled intercurrent illness.
11. History or evidence of clinically significant cardiovascular, CNS, and/or other systemic disease that would, in the investigator's opinion, preclude participation in the study or compromise the participant's ability to give informed consent.
12. Life expectancy <6 months.
13. History or evidence of rare hereditary problems of galactose intolerance, Lapp lactase deficiency, or glucose-galactose malabsorption.
14. Major surgery (excluding lymph node biopsy) within 28 days prior to signing the informed consent form unless the participant is recovered at the time of signing the informed consent form.
15. Any systemic antilymphoma and/or investigational therapy within 28 days prior to the start of Cycle 1.
16. Prior use of lenalidomide in combination with rituximab.
17. History of hypersensitivity to compounds of similar biological or chemical composition to tafasitamab, immunomodulatory drugs, rituximab, other monoclonal antibodies, and/or the excipients contained in the study drug formulations.
18. Any condition that would, in the investigator's judgment, interfere with full participation in the study, including administration of study treatment and attending required study visits; pose a significant risk to the participant; or interfere with interpretation of study data.

### Tafasitamab Regimen:

For the first 3 cycles of the study, each cycle (Cycles 1-3) consists of a tafasitamab 12 mg/kg intravenous infusion on Day 1, Day 8, Day 15, and Day 22 of the cycle. Thereafter, tafasitamab is administered on a bi-weekly basis with 12 mg/kg intravenous infusions on Days 1 and 15 of each repeated 28-day cycle.

### Rituximab Regimen:

The first cycle of the study consists of a rituximab 375 mg/m² intravenous infusion on Days 1, 8, 15, and 22. Thereafter, rituximab is administered as a 375 mg/m² intravenous infusion on Day 1 of every 28-day cycle from Cycle 2 to 5.

### Lenalidomide Regimen:

Participants self-administer 20 mg oral lenalidomide daily on Days 1 through 21 of every 28 day cycle for 12 cycles.

The primary endpoint of the study is progression-free survival by investigator assessment in the follicular lymphoma population. Progression-free survival is defined as the time from randomization to first documented disease progression, or death from any cause, whichever occurs first.

The secondary endpoints of the study include:

1. Progression-free survival by investigator assessment in the overall population (follicular lymphoma and marginal zone lymphoma populations).

2. Positron emission tomography (PET)-complete response rate at end of treatment (4-8 weeks after last treatment) by investigator in the follicular lymphoma population.

3. Overall survival in the follicular lymphoma population.

### EXAMPLE 3

A phase 3, Multicenter, Randomized, Double-Blind, Placebo-Controlled Trial Comparing the Efficacy and Safety of Tafasitamab Plus Lenalidomide in Addition to R-CHOP Cersus R-CHOP in Previously Untreated, High-Intermediate and High-Risk Patients with Newly-Diagnosed Diffuse Large B-Cell Lymphoma (DLBCL)—the Front MIND Study

The safety and preliminary efficacy of tafasitamab with lenalidomide in addition to R-CHOP or tafasitamab in addition to R-CHOP are being tested in the ongoing randomized phase Ib First-MIND trial (NCT04134936) in patients with previously untreated DLBCL (see Example 1). Patients (66) have been randomized, including 24 patients for the safety run-in phase. The data from this safety run-in phase and an additional cut-off with at least 40 patients who completed cycle 1 were reviewed by the independent data safety monitoring board (iDSMB) with a recommendation to continue the study without modification. The safety will be continuously monitored and reviewed by the sponsor and the iDSMB.

This phase 3, multicenter, randomized, double-blind, placebo-controlled trial is designed to compare the efficacy and safety of tafasitamab plus lenalidomide in addition to R-CHOP versus R-CHOP in previously untreated, high-intermediate and high-risk patients with newly-diagnosed DLBCL.

### Clinical Trial Objectives and Endpoints

This double-blind, placebo-controlled, randomized phase 3 MOR208C310 study is designed to investigate whether tafasitamab plus lenalidomide as add-on therapy to R-CHOP will provide improved clinical benefit compared to R-CHOP in previously untreated patients with high-intermediate and high-risk DLBCL.

The primary, secondary and exploratory objectives/endpoints of the clinical trial are presented in Table 10 below.

TABLE 10
Objectives and Endpoints

| | Objective(s) | Corresponding Endpoints |
|---|---|---|
| Primary | To compare the | PFS, defined as the time |
| | efficacy of | from randomization to the |
| | tafasitamab plus | first occurrence of disease |
| | lenalidomide | progression or relapse as |
| | in addition to | assessed by the investigator, |
| | R-CHOP versus | using the Lugano |
| | tafasitamab placebo, | Response Criteria for |
| | lenalidomide | Malignant Lymphoma, or death |
| | placebo and R- | from any cause, whichever |
| | CHOP (henceforth | occurs earlier. |
| | referred to | |
| | as R-CHOP in | |
| | the context of | |
| | the control arm). | |
| Secondary | 1. To compare | Key secondary |
| | the efficacy | a. EFS as assessed by the |
| | (additional | investigator |
| | parameters) of | b. OS |
| | tafasitamab plus | Secondary |
| | lenalidomide in | c. Metabolic, PET- |
| | addition to | negative CR rate at |
| | R-CHOP versus | EOT as assessed by the BIRC |
| | R-CHOP. | d. Metabolic, PET- |
| | | negative CR rate at |
| | | EOT as assessed by |
| | | the investigator |
| | | e. ORR at the EOT |
| | | as assessed by |
| | | the investigator |
| | | f. Time to next anti- |
| | | lymphoma treatment |
| | | (TTNT) |
| | | g. Duration of CR |
| | | as assessed by the |
| | | investigator |
| | | h. MRD status by |
| | | cell-free ctDNA |
| | | assessment at EOT |
| | | i. EFS rate at 3 years |
| | | as assessed by the |
| | | investigator |
| | | j. PFS rate at 3 years |
| | | as assessed by the |
| | | investigator |
| | | k. OS rate at 3 years |
| | 2. To compare | a. Incidence and |
| | the safety of | severity of TEAEs |
| | tafasitamab plus | from the first dose of |
| | lenalidomide | study medication until |
| | in addition to | 30 days after Day 21 |
| | R-CHOP versus | of the last treatment |
| | R-CHOP. | cycle |
| | 3. To compare the | a. PFS as assessed by the |
| | efficacy of | investigator by |
| | tafasitamab plus | COO subtype |
| | lenalidomide in | b. Investigator assessed |
| | addition to | EFS by COO |
| | R-CHOP versus | subtype |
| | R-CHOP in | c. OS by COO subtype |
| | DLBCL subtypes | d. Metabolic, PET- |
| | of different COO | negative CR rate at |
| | assessed by | EOT as assessed by |
| | Hans-classifier | the BIRC by COO |
| | and GEP. | subtype |
| | | e. Metabolic, PET- |
| | | negative CR rate at |
| | | EOT as assessed by the |
| | | investigator by COO |
| | | subtype |
| | 4. To compare | a. PFS as assessed by the |
| | the efficacy of | investigator by locally |
| | tafasitamab plus | determined histological |
| | lenalidomide | subtype |
| | in addition to | b. Investigator |
| | R-CHOP versus | assessed EFS by |
| | R-CHOP in | locally determined |
| | DLBCL subtypes: | histological subtype |
| | DLBCL NOS | c. OS by locally |
| | versus HGBL | determined |
| | versus other. | histological subtype |
| | | d. Metabolic, PET- |
| | | negative CR rate at |
| | | EOT as assessed by |
| | | the BIRC by locally |
| | | determined histological |
| | | subtype |
| | | e. Metabolic, PET- |
| | | negative CR rate at |
| | | EOT as assessed by |
| | | the investigator by |
| | | locally determined |
| | | histological subtype |
| | 5. To compare | 2-year rate of relapse |
| | the incidence | with CNS involvement, as |
| | of CNS relapse | assessed by the investigator. |
| | in patients | |
| | receiving | |
| | tafasitamab plus | |
| | lenalidomide | |
| | in addition to | |
| | R-CHOP versus | |
| | R-CHOP. | |
| | 6. To assess | HRQoL, using the EORTC |
| | PRO in patients | QLQ-C30, and FACT- |
| | receiving | Lym standardized instruments. |
| | tafasitamab plus | |
| | lenalidomide in | |
| | addition to | |
| | R-CHOP versus | |
| | R-CHOP. | |
| | 7. To assess the | Serum concentration of |
| | PK profile of | tafasitamab at specific time |
| | tafasitamab. | points (trough and Cmax levels). |
| | 8. To assess | Incidence of anti-tafasitamab |
| | the potential | antibody formation, |
| | immunogenicity | titer determination of |
| | of tafasitamab. | confirmed positive samples. |
| | 9. To assess | PFS, EFS as assessed by |
| | the role of | the investigator, and OS |
| | baseline NKCC | by baseline NKCC low/high |
| | as a predictor | with a cut-off point for |
| | of response. | NKCC low of ≤115 |
| | | NK cells/μL. |
| Exploratory | 1. To assess | The pharmacodynamic, |
| | the relationship | prognostic or predictive |
| | between potential | value as well as potential |
| | biomarkers and the | resistance to treatment in |
| | efficacy and | relationship with |
| | safety of | selected efficacy or safety |
| | tafasitamab plus | outcomes may be |
| | lenalidomide in | assessed for the following |
| | addition to | biomarkers, e.g.: |
| | R-CHOP versus | a. Peripheral blood B-, |
| | R-CHOP. | T-, and NK-cell count |
| | | b. Peripheral blood cell |
| | | immunophenotyping |
| | | c. Immune cell count |
| | | in the tumor tissue |
| | | d. Gene expression |
| | | profile in the tumor tissue |
| | | e. Semi-quantitative |
| | | CD19 and CD20 |
| | | expression on tumor cells |
| | | f. Genetic mutations |
| | | and genetic subtypes of |
| | | DLBCL |
| | | g. MRD status by cell- |
| | | free ctDNA assessment |
| | | during the study |
| | 2. To compare | Selected primary and |
| | the efficacy of | secondary endpoints |
| | tafasitamab and | (e.g., PFS, EFS, CR |
| | lenalidomide | rate), assessed by |
| | in addition to | BIRC. |
| | R-CHOP by BIRC. | |
| | 3. To assess | HRQoL, using the |
| | PRO in patients | EQ-5D-5L standardized |
| | receiving | questionnaire. |
| | tafasitamab plus | |
| | lenalidomide | |
| | in addition to | |
| | R-CHOP versus | |
| | R-CHOP. | |

Abbreviations: BIRC = blinded independent review committee; Cmax = maximum concentration; CNS = central nervous system; COO = cell of origin; CR = complete response; ctDNA = circulating tumor DNA; DLBCL = diffuse large B-cell lymphoma; EFS = event-free survival; EORTC = European Organisation for the Research and Treatment with of Cancer; EOT = end of treatment; FACT-Lym = Functional Assessment of Cancer Therapy for Patients Lymphoma; GEP = gene expression profiling; HGBL = double-or triple-hit lymphoma; HRQoL = health-related quality of life; MRD = minimal residual disease; NKCC = natural killer cell count; NOS = not otherwise specified; ORR = overall response rate; OS = overall survival; PET = positron emission tomography; PFS = progression-free survival; PK = pharmacokinetics; PRO = patient-reported outcomes; R-CHOP = rituximab-cyclophosphamide, doxorubicin, vincristine, and prednisone; TEAEs = treatment-emergent adverse events.

### Clinical Study Design

### Overall Description of the Study and Investigational Plan

This is a parallel arm, double-blind, placebo-controlled, multi-center, randomized, phase 3 study to investigate the efficacy and safety of tafasitamab plus lenalidomide as add-on therapy to R-CHOP (experimental arm) compared with R-CHOP (control arm) in adult patients with high-intermediate and high-risk patients (defined as IPI3-5 for patients >60 years of age and aaIPI2-3 for patients ≤60 years of age) with newly-diagnosed, previously untreated DLBCL. Approximately 880 patients (440 in each arm) from approximately 350 study centers will be randomized in this study.

For United States only: The sponsor will make efforts to reach the target of approximate 130 patients in this study with the following approximate distribution percentage per ethnic or racial subgroup: White (non-Hispanic): 65%; Hispanic or Latino: 20%; Black or African American: 10%; Asian/Pacific Islander: 5%.

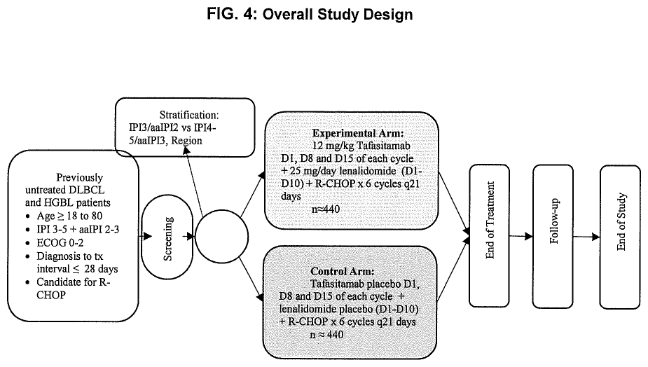

In the experimental arm, patients will receive tafasitamab plus lenalidomide in addition to R-CHOP for six 21-day cycles and in the control arm, patients will receive tafasitamab placebo, lenalidomide placebo and R-CHOP for six 21-day cycles (FIG. 4).

Patients will be randomized in a ratio of 1:1 to either the experimental or control arm. Permutated blocks will be employed using IPI 3 (>60 years old)/aaIPI2 (≤60 years old) vs. IPI 4-5 (>60 years old)/aaIPI 3 (≤60 years old) and geographical regions (Western Europe, United States, Canada, and Australia versus Asia versus the Rest of World [3 groups]) as stratification factors. No crossover to the experimental arm is allowed.

Disease response assessments will be made on PET-CT or PET-MRI according to the Lugano Response Criteria for Malignant Lymphoma (Cheson et al., 2014) and will be based on investigator assessment. Magnetic resonance imaging (MRI) scans may be performed if computed tomography (CT) scans with contrast agent are contraindicated. The scans will include the neck (if involved as baseline), chest, abdomen, and pelvis. If disease in other areas is suspected additional areas should be imaged at all subsequent imaging assessments and/or biopsied (e.g. lumbar puncture).

A safety run-in will be performed with Independent Data Monitoring Committee (IDMC) review after recruitment of 40 randomized patients who have completed at least one treatment cycle (21 days), or prematurely discontinued study treatment. The IDMC will be established to monitor data, to ensure the safety of the patients enrolled in this study and to evaluate the efficacy of the treatment. The IDMC will consist of an independent group of clinical experts who are not involved in the trial management. The IDMC will also review the data at the interim analysis.

Safety will be evaluated by monitoring all AEs, serious adverse events (SAEs), and abnormalities identified through physical examinations, vital signs, and laboratory assessments starting with the date of the informed consent form (ICF) signature. Such events will be graded using the NCI-CTCAE, version 5.0, or higher. Laboratory safety assessments will include routine monitoring of hematology and blood chemistry, and tests of immunologic parameters.

After EOT, patients will enter the up to 60-months treatment follow-up based on visits and an extended follow-up every 6 months afterwards based on telephone contacts until the close of the study.

### Planned Study Analyses and End of Study

The following analyses are pre-planned:
Safety Run-in Analysis: A safety analysis is planned after recruitment of approximately 20 patients per arm who have completed at least one treatment cycle. This analysis will be reviewed by IDMC to monitor the overall safety.
   Interim Analysis: An interim analysis is planned after approximately 100 PFS events as per investigator are observed in the FAS. This analysis will be reviewed by the IDMC to assess futility stop.
   Primary analysis: If the study has not stopped for futility, the primary analysis is planned after approximately 274 PFS events as per investigator are observed.
   Final Analysis: The final analysis will be performed at the end of the study (defined below).

End of Study:
The end of the study is expected to occur approximately 5 years after the first patient is enrolled, to allow all patients to have a minimum of 3 years of follow-up post-treatment. The sponsor has the right to terminate the study at any time.

### Safety Plan

The study will employ safety monitoring activities which will comprise standard evaluation of AEs/SAEs/Adverse Events of Special Interest (AESI) reports (nature, severity, frequency and causality), performance status, physical examinations, ECG and laboratory data assessed on an ongoing basis by the sponsor's responsible safety physicians and/or other nominated personnel to provide support in the review of safety data. Such events will be graded using the NCI-CTCAE, version 5.0, or higher. Laboratory safety assessments will include routine monitoring of hematology and blood chemistry, and tests of immunologic parameters.

Throughout the study, the investigator must record all AEs which occur in a patient from the time that informed consent is obtained until 30 days after Day 21 of the last study treatment cycle, on the AE CRF, regardless of the severity or relationship to study drug. The investigator should treat patients with AEs appropriately and observe them at suitable intervals until the events stabilize or resolve.

An AE will be considered to be a TEAE if it begins or worsens on or after the first dose of study medication, and before the 30th day following the Day 21 of the last study treatment cycle.

### Clinical Trial Duration

The duration of the study is estimated to be approximately 5 years, including 21 months recruitment, 18 weeks treatment and at least 3 years follow-up for the last randomized patient.

Three periods are defined for each patient in the study.

### Screening Period

The screening period of a maximum of 21 days and a minimum of 1 day is the interval between the date of signing of ICF and C1D1. The ICF must be signed prior to beginning any study related assessments. Of note, a PET-CT of suitable quality, an echo or multi-gated acquisition (MUGA) scan, ECG, or virus serology performed as standard of care within 21 days prior to signing the ICF may be used.

During screening, each patient who signs the ICF will be allocated a unique study identification number.

Individuals who do not meet the criteria for participation in the study may be re-screened once. The screening failure rate is expected to be approximately 20%.

The eligibility of a patient must be confirmed by the investigator or designee during the Screening Period. After the eligibility has been checked and confirmed, the patient can be randomized to one of the treatment arms by using the IRT system.

The following electronic case report forms (eCRFs) must be completed for a screen failure patient:
1. Informed Consent
2. Inclusion/Exclusion Criteria
3. Demography
4. AEs (only if the patient experienced an SAE during the screening period after signing the ICF)
5. Withdrawal of informed consent, if applicable
6. Death, if applicable

If a screen failure patient experiences an AE, which does not meet the SAE criteria, details about the AE will be recorded only in the investigator's source documents. In case of an SAE, data must be recorded on both the AE and SAE forms. No other data will be entered into the clinical database for patients who are screen failures. If a patient is randomized as per IRT but fails to start on study treatment, then this must be notified using IRT.

Note: It is important to note that the time from initial pathological diagnosis (as defined by the date of the first biopsy specimen containing lymphoma according to the local pathology report) to C1D1 must be ≤28 days.

### Study Treatment Period

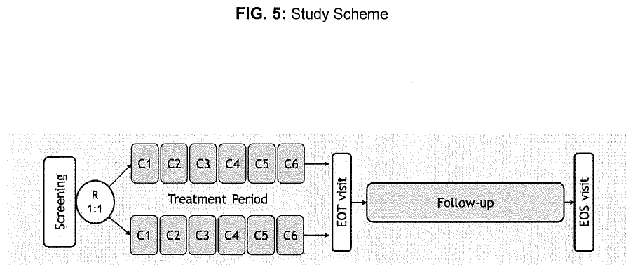

The treatment period starts with the first administration of study drug (C1D1) and consists of 6 cycles, each 21 days (FIG. 5). The EOT visit or Early Treatment Discontinuation (ETD) visit will be performed 6±2 weeks after EOT which is defined as day 21 of the last treatment cycle the patient started. Patients who discontinue early (e.g., because of progression, AEs, etc.) will have an ETD visit. In either case, a PET-scan (PET-CT or PET-MRI) at EOT or early study treatment discontinuation is mandated.

All assessments should be performed within the acceptable visit windows (±2 days) on Day 1, Day 8 and Day 15 of each cycle (Note: C1D1 visit window: +1 day).

### Selection and Withdrawal of Patients

The investigator or designee must ensure that only patients who meet all the following inclusion and none of the exclusion criteria are enrolled in the study. The patients are not allowed to participate in additional parallel investigational drug or device studies. The sponsor is not providing waivers to the clinical trial protocol as deviations might have a negative impact on patient safety or the scientific integrity and regulatory acceptability of the clinical trial.

### Inclusion Criteria:

Patients considered for participation in the clinical trial must meet all of the following criteria:
1. Written informed consent.
2. Age 18 to 80 years at the time of signing the ICF.
3. Previously untreated patients with local biopsy-proven, CD20-positive DLBCL, including one of the following diagnoses by 2016 WHO classification of lymphoid neoplasms are eligible (Swerdlow et al., 2016):
   a. DLBCL, not otherwise specified (NOS) including germinal center B-cell (GCB) type, activated B-cell (ABC) type
   b. T-cell rich large BCL
   c. Epstein-Barr virus-positive DLBCL, NOS
   d. Anaplastic lymphoma kinase (ALK)-positive large BCL
   e. Human Herpes virus-8 (HHV8)-positive DLBCL, NOS
   f. High-grade BCL with MYC and B-cell lymphoma 2 (BCL2) and/or B-cell lymphoma 6 (BCL6) rearrangements (double-hit or triple-hit lymphoma). Please note: Patients must be appropriate candidates for R-CHOP. If an investigator deems a patient with a known double- or triple-hit lymphoma (HGBL) should be treated more aggressively (e.g. dose-adjusted etoposide, prednisone, vincristine, cyclophosphamide, doxorubicin and rituximab [DA-EPOCH-R] or cyclophosphamide, vincristine, doxorubicin and dexamethasone (CVAD) followed by methotrexate and cytarabine [Hyper CVAD]), this patient would not be considered eligible for this study
   g. DLBCL coexistent with either follicular lymphoma (FL) of any grade, gastric MALT lymphoma or non-gastric MALT lymphoma
   h. FL grade 3b
4. Availability of archival or freshly collected tumor tissue sent for retrospective central pathology review. Please note: neither receipt of tumor samples nor central review of diagnosis is necessary prior to study enrollment.
5. Up to six of the largest target nodes, nodal masses, or other lymphomatous lesions that are measurable in two diameters should be identified by local assessment from different body regions representative of the patient's overall disease burden and include mediastinal and retroperitoneal disease, if involved. At baseline, a measurable node must be greater than 15 mm in longest diameter (LDi). Measurable extranodal disease may be included in the six representative, measured lesions. At baseline, measurable extranodal lesions should be greater than 10 mm LDi. All other lesions (including nodal, extranodal, and assessable disease) should be followed as nonmeasured disease as non-target lesions (e.g. cutaneous, GI, spleen, liver, kidneys, pleural or pericardial effusions, ascites, bone, bone marrow). At least one measurable lesion must be confirmed to be PET-positive (Deauville score of 4 or 5) at the time of randomization by local assessment.
6. ECOG performance status of 0, 1, or 2.
7. IPI status of 3 to 5 (for patients >60 years of age) or aaIPI 2 to3 (for patients ≤60 years of age).
8. Diagnosis to treatment interval, defined as the time between the date of DLBCL diagnosis (date of the first biopsy specimen containing lymphoma according to the local pathology report) and the start of treatment (C1D1) ≤28 days.
9. Left ventricular ejection fraction equal to or greater than lower limit of institutional normal range, assessed by local echocardiography or cardiac multi-gated acquisition (MUGA) scan.
10. Patient must have the following local laboratory criteria at screening:
   a. Absolute neutrophil count (ANC) ≥1.5×10⁹/L (unless secondary to bone marrow involvement by DLBCL)
   b. Platelet count ≥75×10⁹/L (unless secondary to bone marrow involvement by DLBCL)
   c. Total serum bilirubin <1.5×upper limit of normal (ULN) unless secondary to Gilbert's Syndrome or documented liver involvement by lymphoma. Patients with Gilbert's Syndrome or documented liver involvement by lymphoma may be included if their total bilirubin is ≤5×ULN
   d. Alanine aminotransferase (ALT), aspartate aminotransferase (AST) and alkaline phosphatase (ALP) ≤3×ULN, or ≤5×ULN in cases of documented liver involvement
   e. Serum creatinine clearance must be ≥30 mL/minute either measured or calculated using a standard Cockcroft and Gault formula (Cockroft and Gault, 1976)
11. In the opinion of investigator, the patient must:
   a. Be able and willing to receive adequate prophylaxis and/or therapy for thromboembolic events, e.g. aspirin 81 to 325 mg daily or low molecular weight heparin. This is due to increased risk of thrombosis in patients treated with lenalidomide without prophylaxis. Patients unable or unwilling to take any prophylaxis are not eligible
   b. Be able to understand, give written informed consent, and comply with all study-related procedures, medication use, and evaluations
   c. Not have a history of noncompliance in relation to medical regimens nor be considered potentially unreliable and/or uncooperative
   d. Be able to understand the reason for complying with the special conditions of the pregnancy prevention risk management plan and in writing acknowledge to adhere to this plan
12. Due to the teratogenic potential of lenalidomide, females of childbearing potential (FCBP) must:
Applicable in all Countries Except US:
   i. Not be pregnant as confirmed by a negative serum pregnancy test at screening and a medically supervised urine pregnancy test prior to starting study therapy
   j. Refrain from breast feeding and donating oocytes during the course of study and for 3 months after the last dose of study drug or according to local guidelines for R-CHOP, whichever is longer
   k. Agree to ongoing pregnancy testing during the course of the study and after study therapy has ended. This applies even if the patient applies complete sexual abstinence
   l. Commit to continued abstinence from heterosexual intercourse if it is in accordance with her lifestyle (which must be reviewed on a monthly basis) or agree to use and be able to comply with the use of highly effective contraception without interruption at least 4 weeks prior to start of study drugs, during the study treatment and for 3 months after the last dose of study drug, or, for R-CHOP, according to the local guidelines, whichever is longer.
Applicable in US:
   m. Not be pregnant as confirmed by pregnancy tests performed before treatment initiation, within 10-14 days and again within 24 hours of initiating treatment (even if true abstinence is the chosen method of birth control)
   n. Refrain from breast feeding and donating oocytes during the course of study and for 3 months after the last dose of study drug, or according to US guidelines for R-CHOP, whichever takes longer
   o. Agree to ongoing pregnancy testing during the course of the study (every 3 weeks in women with regular menstrual cycle and every 2 weeks in women with irregular menstrual cycle), and after study therapy has ended (even if true abstinence is the chosen method of birth control)
   p. Not get pregnant while taking the study drug and for at least 3 months after the last dose of study drugs by using at the same time 2 effective methods of contraception, each time engaging in sexual activity with a male, starting at least 4 weeks before taking the study drug, while taking the study drug, during breaks (dose interruptions) and for at least 3 months after stopping the study drug, or for R-CHOP, according to the US guidelines, whichever is longer. True abstinence from heterosexual sexual intercourse is also an acceptable method of contraception. The use of emergency contraception is also permitted
13. Male participants must:
Applicable in all Countries Except US:
   b. Use an effective barrier method of contraception without interruption if the patient is sexually active with FCBP. Male participants should refrain from donating sperm during the study participation and for 3 months after the last dose of study drug, or according to the local guidelines for R-CHOP, whichever is longer
Applicable in US:
   b. Use a latex or synthetic condom each time they have sex with a FCBP. True abstinence from heterosexual sexual intercourse is also an acceptable method of contraception. The use of emergency contraception is also permitted. Male participants should refrain from donating sperm during the study participation and for 3 months after the last dose of study drug, or according to the US guidelines for R-CHOP, whichever is longer

### Exclusion Criteria:

Patients must be excluded from participating in this clinical trial if they meet any of the following criteria:
1. Any other histological type of lymphoma according to WHO 2016 classification of lymphoid neoplasms, e.g. primary mediastinal (thymic) large B-cell lymphoma, Burkitt's lymphoma, BCL, unclassifiable, with features intermediate between DLBCL and classical Hodgkin lymphoma (grey-zone lymphoma); primary effusion lymphoma; primary cutaneous DLBCL, leg type; primary DLBCL of the CNS; DLBCL arising from CLL or indolent lymphoma.
2. History of radiation therapy to ≥25% of the bone marrow for other diseases.
3. History of prior non-hematologic malignancy except for the following:
   a. Malignancy treated with curative intent and with no evidence of active disease present for more than 2 years before screening
   b. Adequately treated lentigo maligns melanoma without current evidence of disease or adequately controlled non-melanomatous skin cancer.
   c. Adequately treated carcinoma in situ without current evidence of disease
4. Patients with:
   a. Positive local test result during screening for hepatitis C (hepatitis C virus [HCV] antibody serology testing) and a positive test for HCV RNA. Patients with positive serology must have been tested locally for HCV RNA and are eligible, in case of negative HCV RNA test results
   b. Positive local test result during screening for chronic hepatitis B virus (HBV) infection (defined by hepatitis B surface antigen [HBsAg] positivity). Patients with occult or prior HBV infection (defined as negative HBsAg and positive total hepatitis B core antibody [HBcAb]) may be included if HBV DNA was undetectable (local test result), provided that they are willing to undergo ongoing DNA testing. Antiviral prophylaxis may be administered as per institutional guidelines. Patients who have protective titers of hepatitis B surface antibody (HBsAb) after vaccination or prior but cured hepatitis B are eligible
   c. Seropositive (local test during screening) for, or history of active viral infection with human immunodeficiency virus (HIV)
   d. Known active systemic bacterial, viral, fungal, or other infection at screening, including patients with suspected active or latent tuberculosis (as confirmed by a positive interferon-gamma release assay)
   e. Positive results for the human T-lymphotrophic 1 virus (HTLV-1). HTLV testing during screening is required for patients at sites in endemic countries (Japan and Melanesia and countries in the Caribbean basin, South America, Central America, and sub-Saharan Africa)
   f. Known CNS lymphoma involvement
   g. History or evidence of clinically significant cardiovascular, CNS and/or other systemic disease that would in the investigator's opinion preclude participation in the study or compromise the patient's ability to give informed consent
   h. History or evidence of rare hereditary problems of galactose intolerance, Lapp lactase deficiency or glucose-galactose malabsorption
   i. Vaccination with live vaccine within 21 days prior to study randomization
   j. Major surgery within up to 21 days prior to signing the ICF, unless the patient is recovered at the time of signing the ICF
   k. Any systemic anti-lymphoma and/or investigational therapy prior to the start of C1D1, except for permitted pre-phase treatment.
   l. Contraindication to any of the individual components of R-CHOP, including prior receipt of anthracyclines
   m. Pregnancy or lactation
   n. History of hypersensitivity to any component of R-CHOP, to lenalidomide, to compounds of similar biological or chemical composition to tafasitamab, IMiDs® and/or the excipients contained in the study drug formulations

### Treatment of Patients

Each investigator is responsible for ensuring that deliveries of investigational medicinal product/s (IMP/s) and other clinical trial materials from the sponsor are completely and correctly received, recorded, handled and stored safely and properly in accordance with all applicable regulatory guidelines, and used in accordance with this clinical trial protocol and related plans.

### Pre-Phase Treatment

For patients in urgent need of pre-phase treatment before initiation of study treatment at C1D1, the use of steroids (e.g. oral prednisone 25 to 100 mg/d or equivalent) for a maximum of 7 days with or without rituximab (375 mg/m²) or vincristine (e.g. 1 mg) is allowed after tumor investigations (imaging, blood samples) for screening have been performed.

The baseline PET/CT or PET/MRI assessment must be performed prior to administration of corticosteroids, rituximab or vincristine. Only under exceptional circumstances, and at the discretion of the investigator, the pre-phase corticosteroid treatment may be started prior to the baseline PET/CT or PET/MRI assessment.

Any pre-phase treatment must be adequately documented and justified in the patient's source data and in the eCRF. Note: if rituximab or vincristine is administered as part of pre-phase treatment, it must be omitted on C1D1.

### Study Treatment Phase

A complete treatment cycle is defined as 21 calendar days during which tafasitamab plus lenalidomide in addition to R-CHOP (experimental arm) or tafasitamab placebo (0.9% saline solution), lenalidomide placebo and R-CHOP (control arm) will be administered according to the following plan.

Pre-Medication for Tafasitamab/Placebo Infusions—IRR prophylaxis

Tafasitamab/placebo infusions should be administered to well-hydrated patients after pre-medication with oral acetaminophen (e.g. 650-1000 mg), an antihistamine such as diphenhydramine hydrochloride (50-100 mg) and glucocorticosteroids (e.g. 100 mg IV prednisone or prednisolone or equivalent) 30-60 minutes prior to starting in cycle 1. Note: the Day 1 steroid dose being part of CHOP (100 mg prednisone or prednisolone or equivalent, IV or PO) can be used as a component of premedication prior to tafasitamab/placebo infusion.

Premedication is mandatory for the first cycle (Day 1, Day 8, Day 15). For patients who do not experience Grade ≥2 IRRs/≥Grade 1 cytokine release syndrome (CRS) to tafasitamab/placebo during the first cycle, premedication will be optional for subsequent antibody/placebo infusions at the discretion of the investigator. Otherwise, the premedication should be continued for subsequent administrations.

Pre-Medication for Tafasitamab/Placebo and Rituximab Infusions after occurrence of Allergic Reaction or Grade 2 Hypersensitivity

For subsequent cycles, premedication for tafasitamab/placebo and rituximab should include oral acetaminophen (e.g. 650-1000 mg), an antihistamine such as diphenhydramine hydrochloride (50-100 mg) and glucocorticosteroids (e.g. 100 mg IV prednisone or prednisolone or equivalent) 30-60 minutes prior to the infusion.

### Experimental Arm: Tafasitamab Plus Lenalidomide in Addition to R-CHOP

Study treatment consisting of tafasitamab plus lenalidomide and R-CHOP will be in six 21-day cycles (see Table 11).

TABLE 11
Treatment with Tafasitamab plus Lenalidomide in Addition to R-CHOP

| | | Dosing days |
|---|---|---|
| Drug | Dose | (21 day cycle) |
| Tafasitamab | 12 mg/kg IV | 1, 8, 15 |
| Lenalidomide* | 25 mg/day PO | 1-10 |
| Rituximab or a locally | 375 mg/m² IV | 1 |
| approved biosimilar** | | |
| Cyclophosphamide | 750 mg/m² IV | 1 |
| Doxorubicin | 50 mg/m² IV | 1 |
| Vincristine | 1.4 mg/m² (max 2.0 mg | 1 |
| | total) IV | |
| Prednisone/Prednisolone | 100 mg/day PO | 1-5 |

Abbreviations: IV = intravenous; PO = per os.
*Lenalidomide: Patients will self-administer a starting dose of 25 mg oral lenalidomide daily on Days 1-10 of each 21-day cycle. Dose modification due to toxicity is permitted in 5 mg steps in each cycle. The minimum dose of lenalidomide is 10 mg.
**Only one rituximab IV product should be used for one patient throughout the study, if possible.

### Control Arm: Tafasitamab Placebo and Lenalidomide Placebo in Addition to R-CHOP

Study treatment consisting of tafasitamab placebo and lenalidomide placebo in addition to R-CHOP will be in six 21-day cycles (see Table 12 below).

TABLE 12
Treatment with Tafasitamab Placebo and Lenalidomide
Placebo in Addition to R-CHOP

| | | Dosing days |
|---|---|---|
| Drug | Dose | (21 day cycle) |
| Tafasitamab placebo (0.9% | na | 1, 8, 15 |
| saline solution) | | |
| Lenalidomide placebo | na | 1-10 |
| Rituximab or a locally | 375 mg/m² IV | 1 |
| approved biosimilar* | | |
| Cyclophosphamide | 750 mg/m² IV | 1 |
| Doxorubicin | 50 mg/m² IV | 1 |
| Vincristine | 1.4 mg/m² (max | 1 |
| | 2.0 mg total) IV | |
| Prednisone/Prednisolone | 100 mg/day PO | 1-5 |

Abbreviations: IV = intravenous, PO = per os.
*Only one rituximab IV product should be used for one patient throughout the study, if possible.

Note: All components of the study treatment should start on the same day but may be administered over 2 days as a maximum (e.g. tafasitamab/placebo infusion on Day 1 and R-CHOP start at Day 2). Day 1 is defined of the start of any study treatment component. Within a cycle, the administration of tafasitamab/placebo may be shifted for ±2 days maximum. Whenever possible, the dosing interval of 21 days between R-CHOP administrations should be followed to maintain R-CHOP dose intensity. Every dose delay and dose modifications of any study drug component need to be documented in the eCRF.

### Efficacy Analysis

All efficacy analyses will be performed to compare the experimental arm (tafasitamab+lenalidomide in addition to R-CHOP) vs. control arm (tafasitamab placebo+lenalidomide placebo in addition to R-CHOP) in FAS.

For all stratified analyses, the strata information will be based on the data obtained from the IRT that was utilized for randomization.

### Testing Strategy of Primary and Key Secondary Endpoints

The primary endpoint for this study is PFS as per investigator.

The key secondary endpoints are:
1. EFS as assessed by the investigator
2. OS

If statistical significance is met for the primary endpoint, then the key-secondary endpoints will be tested in the FAS hierarchically in the order mentioned above.

During the time of primary analysis when 274 PFS events as per investigator are observed, it is expected to observe 311 EFS events and 245 OS events.

An interim OS analysis will be performed at the time of the primary analysis when 274 PFS events as per investigator have been observed and a final OS analysis will be performed at the end of the study.

The interim and final OS analysis will be performed only if the primary and the other key secondary endpoint have passed the 2-sided 5% significance level. Group sequential methods using an alpha-spending function with an O'Brien-Fleming boundary will be used to control the type I error at the 0.05 level. The details of the statistical tests for the primary and the key secondary endpoints are discussed below.

### Primary Endpoint

As stated above, the primary efficacy endpoint is PFS as determined by the investigator, defined as the time from the date of randomization until the first occurrence of disease progression or relapse as assessed by the investigator using the 2014 Lugano classification criteria for Malignant Lymphoma (Cheson et al. 2014), or death from any cause, whichever occurs earlier.

For patients who have not progressed, relapsed, or died as of the clinical cutoff date for analysis, PFS will be censored on the date of last disease assessment when the patient is known to be progression-free. If no tumor assessments are performed after the baseline visit or all post-baseline tumor assessment results have overall responses of “not evaluable,” PFS will be censored on the date of randomization. If the patient starts a new anti-lymphoma treatment (medication, radiotherapy or surgery), the censoring date is the date of the last adequate tumor assessment before the initiation of the new anti-lymphoma treatment, or before the cut-off date, whichever comes first.

The date of last adequate tumor assessment is the date of the last tumor assessment with overall response of CR, PR, SD. In this case, the last tumor evaluation date at that assessment is used.

If disease progression is documented after two or more missing or non-adequate tumor assessments, then the date of PFS will be censored at the date of the last tumor assessment with overall response of CR, PR, or SD.

The primary analysis of the study will test the equality of PFS distributions in the treatment arm vs. the control arm:

H₀: PFS_{Experimental Arm}=PFS_{Control Arm} versus H_{A}: PFS_{Experimental Arm}≠PFS_{Control Arm}.

### Secondary Endpoints

TABLE 13
Key Secondary Endpoints

| Type of | | | Type of |
|---|---|---|---|
| Endpoint | Endpoint | Definition | Endpoint |
| Key | EFS-INV | EFS-INV is defined as | Time to |
| Secondary- | | the time from the date | Event |
| 1 | | of randomization until the first | |
| | | occurrence of disease progression | |
| | | or relapse as assessed | |
| | | by the INV using the | |
| | | 2014 Lugano classification criteria | |
| | | for Malignant Lymphoma | |
| | | (Cheson et al. 2014), start | |
| | | of new anti-lymphoma treatment | |
| | | (including medication, radiation or | |
| | | surgery) or death from any cause, | |
| | | whichever occurs first. | |
| | | Patients who do not have a record | |
| | | of disease progression | |
| | | or relapse, initiation | |
| | | of a new non-study | |
| | | anti-DLBCL treatment | |
| | | or death from any cause | |
| | | will be censored | |
| | | on the date of last available | |
| | | response assessment, or at | |
| | | randomization date if no | |
| | | post-baseline assessment | |
| | | is available. | |
| Key | OS | OS is defined as the | Time to |
| Secondary- | | period from the date | Event |
| 2 | | of randomization until | |
| | | the date of death | |
| | | from any cause. For | |
| | | patients who have not | |
| | | died at the cutoff date | |
| | | for analysis, OS will be | |
| | | censored on the last date when the | |
| | | patients are known to be alive. | |

Abbreviations: DLBCL = diffuse large B-cell lymphoma; EFS = event free survival; INV = investigator; OS = overall survival.

TABLE 14
Other Secondary Endpoints

| Type of | | | Type of |
|---|---|---|---|
| Endpoint | Endpoint | Definition | Endpoint |
| Secondary- | Metabolic | The metabolic PET- | Binary |
| 1 | PET- | negative CR rate is defined | |
| | negative | as the proportion of | |
| | CR-rate at | patients who achieved | |
| | EOT by | metabolic PET-negative | |
| | BIRC | CR as per Lugano | |
| | | 2014 criteria based on | |
| | | PET/CTs performed at | |
| | | the end of the treatment | |
| | | by BIRC. | |
| Secondary- | Metabolic | The metabolic PET-negative | Binary |
| 2 | PET- | CR rate is defined | |
| | negative | as the proportion of | |
| | CR-rate at | patients who achieved | |
| | EOT by | metabolic PET-negative | |
| | INV | CR as per Lugano | |
| | | 2014 criteria based on | |
| | | PET/CTs performed at | |
| | | the end of the treatment | |
| | | by the INV. | |
| Secondary- | MRD | MRD status by cell-free | Binary |
| 3 | status at | ctDNA assessment at | |
| | EOT | EOT. | |
| Secondary- | PFS at 3 | PFS rate calculated | KM |
| 4 | years | through Kaplan-Meier | based |
| | | method at 36 months | Binary |
| | | after randomization. | |
| | | For this endpoint, tumor | |
| | | response as per INV | |
| | | will be considered. | |
| Secondary- | EFS at 3 | EFS rate calculated | KM |
| 5 | years as | through Kaplan-Meier | based |
| | per | method at 36 months | Binary |
| | INV | after randomization. | |
| | | For this endpoint, tumor | |
| | | response as per INV | |
| | | will be considered. | |
| Secondary- | OS at 3 | OS rate calculated | KM |
| 6 | years | through Kaplan-Meier | based |
| | | method at 36 months | Binary |
| | | after randomization. | |
| Secondary- | ORR | The ORR is defined | Binary |
| 7 | as per | as the proportion of | |
| | INV | patients with CR or | |
| | at EOT | PR as per Lugano 2014 | |
| | | criteria based on | |
| | | assessment at the end of | |
| | | the treatment by the INV. | |
| Secondary- | DoCR | Duration of CR is defined | Time to |
| 8 | as per | as the time from the | event |
| | INV | date of the first occurrence | |
| | | of a documented | |
| | | CR, to the date of | |
| | | progression, relapse, death | |
| | | from any cause or start | |
| | | of next anti-lymphoma | |
| | | treatment whichever is | |
| | | earlier for the subgroup | |
| | | of patients with a | |
| | | BOR of CR. | |
| | | For patients achieving a | |
| | | CR but who have not | |
| | | progressed, relapsed, | |
| | | died or started a new | |
| | | anti-lymphoma treatment | |
| | | at the time of analysis, | |
| | | DoCR will be censored | |
| | | on the date of last tumor | |
| | | assessment when the | |
| | | patient is known to be | |
| | | progression-free. | |
| | | For this endpoint, tumor | |
| | | response as per INV | |
| | | will be considered. | |
| Secondary- | TTNT | TTNT is defined as the | Time to |
| 9 | | time from randomization | Event |
| | | date to start of next anti- | |
| | | lymphoma therapy (for | |
| | | any reason including | |
| | | disease progression, | |
| | | treatment toxicity and | |
| | | patient preference) or | |
| | | death due to any cause, | |
| | | whatever comes first. | |
| | | Patients without | |
| | | documented institution of a | |
| | | new anti-neoplastic | |
| | | therapy or death until the | |
| | | cut-off date will be | |
| | | censored at the date of last | |
| | | contact prior to the | |
| | | cut-off date. | |
| Secondary- | 2-year | 2-year rate of relapse | KM |
| 10 | rate of | with CNS involvement, as | based |
| | CNS | assessed by the INV. | Binary |
| | relapse | | |
| Secondary- | EORTC | Time-to-deterioration in | Time to |
| 11 | QLQ- | EORTC QLQ-C30 | Event |
| | C30 | physical functioning | Endpoint |
| | | and fatigue. | |
| | | For EORTC QLQ-C30 | |
| | | physical functioning and | |
| | | fatigue, clinically | |
| | | meaningful deterioration is | |
| | | defined as a ≥10-point | |
| | | decrease and increase, | |
| | | respectively, from baseline | |
| | | (Osoba et al. 1998; | |
| | | Cocks et al., 2012). | |
| | | Patients without occurrence | |
| | | of deterioration at | |
| | | the clinical cutoff date | |
| | | will be censored at the | |
| | | last available EORTC | |
| | | QLQ-C30 assessment. | |
| | | Proportion of patients in | Binary |
| | | each arm achieving | Endpoint |
| | | clinically meaningful | |
| | | improvement in EORTC | |
| | | QLQ-C30 physical | |
| | | functioning and fatigue at | |
| | | any time in the study. | |
| | | For EORTC QLQ-C30 | |
| | | physical functioning and | |
| | | fatigue, clinically | |
| | | meaningful improvement is | |
| | | defined as a ≥7-point | |
| | | increase and ≥9-point | |
| | | decrease, respectively, | |
| | | from baseline (Cocks et | |
| | | al., 2012). | |
| | | Comparison of EORTC | Con- |
| | | QLQ-C30 treatment- | tinuous |
| | | related symptoms between | |
| | | two treatment arms. | |
| Secondary- | FACT- | Time-to-deterioration. | Time to |
| 12 | Lym | For the FACT-Lym LymS, | Event |
| | | clinically meaningful | |
| | | deterioration is defined | |
| | | as ≥3-point decrease | |
| | | from baseline (Carter et al., | |
| | | 2008; Hlubocky et | |
| | | al., 2013). | |
| | | Patients without occurrence | |
| | | of deterioration at | |
| | | the clinical cutoff date | |
| | | will be censored at the | |
| | | last available FACT-Lym | |
| | | assessment. | |
| | | Proportion of patients | Binary |
| | | in each arm achieving | |
| | | clinically meaningful | |
| | | improvement at any time in | |
| | | the study. | |
| | | For the FACT-Lym LymS, | |
| | | clinically meaningful | |
| | | improvement is defined | |
| | | as ≥3-point increase | |
| | | from baseline (Carter et al., | |
| | | 2008; Hlubocky et | |
| | | al., 2013). | |
| | | Comparison of FACT-Lym | Con- |
| | | treatment-related | tinuous |
| | | symptoms between two | |
| | | treatment arms. | |

Abbreviations: BIRC = blinded independent review committee; BOR = best overall response; CNS = central nervous system; CR = complete response; CT = computer tomography; EOT = end of treatment; ctDNA = circulating tumor DNA; DFS = disease-free survival; DLBCL = diffuse large B-cell lymphoma; DoCR = durability of complete response; EFS = event free survival; EORTC = European Organisation for the Research and Treatment of Cancer; EOT = end of treatment; FACT-Lym = functional assessment of cancer therapy for patients with lymphoma; LymS = Iymphoma subscale. INV = investigator; IRC = Independent review committee; KM = Kaplan-Meier; MRD = minimal residual disease; ORR = overall response rate; OS = overall survival; PET = positron emission tomography; PFS = progression-free survival; TTNT = time to next anti-lymphoma treatment.

### Exploratory Endpoints

TABLE 15
Overview of Exploratory Endpoints

| Exploratory Endpoints | Definition/Analysis |
|---|---|
| Exploratory Biomarker Analysis | Exploratory analyses of |
| for the following biomarkers: | biomarkers will be |
| a. Peripheral blood B-, T-, | conducted. Analyses |
| and NK-cell count | will assess the |
| b. Peripheral blood cell | pharmacodynamics, prognostic |
| immunophenotyping | and/or predictive |
| c. Immune cell count in | value of candidate biomarkers. |
| the tumor tissue | The association |
| d. Gene expression profile | between candidate biomarkers |
| in the tumor tissue | with selected |
| e. Semi-quantitative | measures of efficacy and |
| CD19 and CD20 | safety, with treatment |
| expression on tumor cells | and independent of treatment, |
| f. Genetic mutations and genetic | will be explored. |
| subtypes of DLBCL | |
| g. MRD status by cell-free ctDNA | |
| assessment during the study | |
| PFS-BIRC | PFS as determined by the |
| | BIRC, defined as the |
| | time from the date of |
| | randomization until the first |
| | occurrence of disease |
| | progression or relapse as |
| | assessed by the BIRC |
| | using the 2014 Lugano |
| | classification criteria for |
| | Malignant Lymphoma |
| | (Cheson et al. 2014), or |
| | death from any cause, |
| | whichever occurs earlier. |
| EFS-BIRC | EFS-BIRC is defined as |
| | the time from the date of |
| | randomization until the |
| | first occurrence of disease |
| | progression or relapse as |
| | assessed by BIRC using |
| | the 2014 Lugano |
| | classification criteria for |
| | Malignant Lymphoma |
| | (Cheson et al. 2014), start |
| | of new anti-lymphoma |
| | treatment (including |
| | medication, radiation or |
| | surgery) or death from any |
| | cause, whichever occurs first. |
| | Patients who do not have |
| | a record of disease |
| | progression or relapse, |
| | initiation of a new non- |
| | study anti-DLBCL treatment |
| | or death from any |
| | cause will be censored |
| | on the date of last |
| | adequate tumor assessment, |
| | or at randomization |
| | date if no post-baseline |
| | assessment is available. |
| EQ-5D-5L | There are two components |
| | to the EQ-5D-5L: a |
| | five-item health state |
| | profile that assesses |
| | mobility, self-care, |
| | usual activities, pain or |
| | discomfort, and anxiety |
| | or depression, and a visual |
| | analogue scale that |
| | measures overall health state. |
| | Published weighting |
| | systems allow for creation of |
| | a single composite score |
| | of the patient's health |
| | status. |

Abbreviations: ctDNA = circulating tumor DNA; DLBCL = diffuse large B-cell lymphoma; MRD = minimal residual disease; NKCC = natural killer cell count.

### Subgroup Analysis for Efficacy Endpoints

Subgroup Analysis will be performed for the following endpoints for the following subgroups (Table 16):

TABLE 16
Overview of Subgroup Analysis of Efficacy Endpoints

| Endpoints | Pre-selected Subgroups |
|---|---|
| 1. PFS-INV | 1. COO subtype: |
| 2. EFS-INV | a. Based on IHC/Hans algorithm |
| 3. OS | (GCB vs Non-GCB) |
| | b. Based on GEP (GCB vs ABC vs |
| | non classifiable) |
| | 2. Histological subtypes (DLBCL NOS vs |
| | HGBL vs other subtypes). |
| | 3. High peripheral blood NK-cell count vs low |
| | peripheral blood NK-cell count defined as |
| | ≤115 NK cells/μL |
| | 4. Type of rituximab product. |
| | 5. Age (<65 years vs ≥65 years). |
| | 6. Gender (male vs female). |
| | 7. Race. |
| | 8. Ethnicity. |
| | 9. Stratification factor IPI 3/aaIPI 2 vs. IPI 4- |
| | 5/aaIPI. |
| | 10. Stratification factor geographic region |
| | (Western Europe, United States, Canada, |
| | and Australia vs. either Asia or vs. the |
| | Rest of World [3 groups]). |

Abbreviations: aaIPI = age-adjusted international prognostic index; COO = cell of origin; DLBCL = diffuse large B-cell lymphoma; EFS = event free survival; GCB = germinal center B-Cell type; HGBL = double- or triple-hit lymphoma; IHC = immunohistochemistry; INV = investigator; IPI = international prognostic index; NOS = not otherwise specified; OS = overall survival; PFS = progression-free survival.

For the subgroup analysis, the analysis of the endpoint will be repeated within the subgroups, followed by a model that also includes the treatment-by-subgroup interaction.

### REFERENCES

Boltezar L, Prevodnik V K, Perme M P et al. Comparison of the algorithms classifying the ABC and GCB subtypes in diffuse large B-cell lymphoma. Oncol Lett. 2018; 15(5):6903-6912.
Carbone P P, Kaplan H S, Musshoff K, et al. Report of the committee on Hodgkin's disease staging classification. Cancer Res. 1971; 31(11):1860-1861.
Cheson B D, Fisher R I, Barrington S F, et al. Recommendations for Initial Evaluation, Staging, and Response Assessment of Hodgkin and Non-Hodgkin Lymphoma: The Lugano Classification. J Clin Oncol. 2014; 32(27):3059-3068.
Choi C H, Park Y H, Lim J H, et al. Prognostic Implication of Semi-quantitative Immunohistochemical Assessment of CD20 Expression in Diffuse Large B-Cell Lymphoma. J Pathol Transl Med. 2016; 50(2):96-103.
Cockcroft D W, Gault M R Prediction of creatinine clearance from serum creatinine. Nephron. 1976; 16(1):31-41.
Coiffier B, Thieblemont C, Van Den Neste E, et al. Long-term outcome of patients in the LNH-98.5 trial, the first randomized study comparing rituximab-CHOP to standard CHOP chemotherapy in DLBCL patients: a study by the Groupe d'Etudes des Lymphomes de l'Adulte. Blood. 2010; 116(12):2040-2045.
Cunningham D, Hawkes E A, Jack A, et al. Rituximab plus cyclophosphamide, doxorubicin, vincristine, and prednisolone in patients with newly diagnosed diffuse large B-cell non-Hodgkin lymphoma: a phase 3 comparison of dose intensification with 14-day versus 21-day cycles. Lancet. 2013; 381(9880):1817-1826.
Czuczman M S, Trněný M, Davies A, et al. A Phase 2/3 Multicenter, Randomized, Open-Label Study to Compare the Efficacy and Safety of Lenalidomide Versus Investigator's Choice in Patients with Relapsed or Refractory Diffuse Large B-Cell Lymphoma. Clin Cancer Res. 2017; 23(15):4127-4137.
Delarue R, Tilly H, Mounier N, et al. Dose-dense rituximab-CHOP compared with standard rituximab-CHOP in elderly patients with diffuse large B-cell lymphoma (the LNH03-6B study): a randomised phase 3 trial. Lancet Oncol. 2013; 14(6):525-533.
Flowers C R, Sinha R, Vose J M. Improving outcomes for patients with diffuse large B-cell lymphoma. CA Cancer J Clin. 2010; 60(6):393-408.
Ginaldi L, De Martinis M, Matutes E, et al. Levels of expression of CD19 and CD20 in chronic B cell leukaemias. J Clin Pathol. 1998; 51(5):364-369.
Habermann T M, Weller E A, Morrison V A, et al. Rituximab-CHOP versus CHOP alone or with maintenance rituximab in older patients with diffuse large B-cell lymphoma. J Clin Oncol. 2006; 24(19):3121-3127.
Hammer O. CD19 as an attractive target for antibody-based therapy. MAbs. 2012; 4(5):571-577.
Johnson N A, Boyle M, Bashashati A, et al. Diffuse large B-cell lymphoma: reduced CD20 expression is associated with an inferior survival. Blood. 2009; 113(16):3773-3780.
Jurczak W, Zinzani P L, Gaidano G, et al. Phase IIa study of the CD19 antibody MOR208 in patients with relapsed or refractory B-cell non-Hodgkin's lymphoma. Ann Oncol. 2018; 29(5):1266-1272.
Klisovic R, Winderlich M, Ambarkhane S, et al. Single-agent MOR208 in patients with relapsed/refractory (R/R) B-cell acute lymphoblastic leukemia (B-ALL): a single-arm phase II study. EHA Library (conference poster). 2017; 180615:E839.
Lenz G, Wright G, Dave S S, et al. Stromal gene signatures in large-B-cell lymphomas. N Engl J Med. 2008; 359(22):2313-2323
Lister T A, Crowther D, Sutcliffe S B, et al. Report of a committee convened to discuss the evaluation and staging of patients with Hodgkin's disease: Cotswolds meeting. J Clin Oncol. 1989; 7(11):1630-1636.
Nowakowski G S, LaPlant B, Habermann T M, et al. Lenalidomide can be safely combined with R-CHOP (R2CHOP) in the initial chemotherapy for aggressive B-cell lymphomas: phase I study. Leukemia. 2011; 25(12):1877-1881.
Nowakowski G S, Hong F, Scott D W, et al. Addition of lenalidomide to R-CHOP (R2CHOP) improves outcomes in newly diagnosed diffuse large B-cell lymphoma (DLBCL): First report of ECOG-ACRIN1412 A randomized Phase 2 US Intergroup study of R2CHOP vs R-CHOP. Hematol Oncol. 2019; 37(S2)37-38.
Oken M M, Creech R H, Tormey D C, et al. Toxicity and response criteria of the Eastern Cooperative Oncology Group. Am J Clin Oncol. 1982; 5(6):649-55.
Olejniczak S H, Stewart C C, Donohue K, et al. A quantitative exploration of surface antigen expression in common B-cell malignancies using flow cytometry. Immunol Invest. 2006; 35(1):93-114.
Pfreundschuh M, Schubert J, Ziepert M, et al. Six versus eight cycles of bi-weekly CHOP-14 with or without rituximab in elderly patients with aggressive CD20+ B-cell lymphomas: a randomised controlled trial (RICOVER-60). Lancet Oncol. 2008; 9(2):105-116.
Salles G A, Johannes DueII J, Eva Gonzalez-Barca E, et al. Single-Arm Phase II Study of MOR208 Combined with Lenalidomide in Patients with Relapsed or Refractory Diffuse Large B-Cell Lymphoma: L-Mind. Blood. 2018; 132(S1):227.
Schmitz R, Wright G W, Huang D W, et al. Genetics and Pathogenesis of Diffuse Large B-Cell Lymphoma. N Engl J Med. 2018; 378(15):1396-1407.
Sehn L H, Congiu A G, Culligan D J, et al. No Added Benefit of Eight Versus Six Cycles of CHOP When Combined with Rituximab in Previously Untreated Diffuse Large B-Cell Lymphoma Patients: Results from the International Phase III GOYA Study. Blood. 2018; 132(S1):783-783.
Shipp M A, Harrington D P, Anderson J R, et al. A predictive model for aggressive non-Hodgkin's lymphoma. N Engl J Med. 1993; 329(14):987-994.
Thieblemont C, Tilly H, da Silva M G, et al. First Analysis of an International Double-Blind Randomized Phase Ill Study of Lenalidomide Maintenance in Elderly Patients with DLBCL Treated with R-CHOP in First Line, the Remarc Study from Lysa. Blood. 2016; 128(22):471-471.
Tokunaga T, Tomita A, Sugimoto K, et al. De novo diffuse large B-cell lymphoma with a CD20 immunohistochemistry-positive and flow cytometry-negative phenotype: molecular mechanisms and correlation with rituximab sensitivity. Cancer Sci. 2014; 105(1):35-43.
Vaidya R, Witzig T E. Prognostic factors for diffuse large B-cell lymphoma in the R(X)CHOP era. Ann Oncol. 2014; 25(11):2124-2133.
Vitolo U, Chiappella A, Franceschetti S, et al. Lenalidomide plus R-CHOP21 in elderly patients with untreated diffuse large B-cell lymphoma: results of the REAL07 open-label, multicentre, phase 2 trial. Lenalidomide plus R-CHOP21 in elderly patients with untreated diffuse large B-cell lymphoma: results of the REAL07 open-label, multicentre, phase 2 trial. Lancet Oncol. 2014; 15(7):730-737.
Vitolo U, Trněný M, Belada D, et al. Obinutuzumab or Rituximab Plus Cyclophosphamide, Doxorubicin, Vincristine, and Prednisone in Previously Untreated Diffuse Large B-Cell Lymphoma. J Clin Oncol. 2017; 35(31):3529-3537.
Vitolo U, Witzig T E, Gascoyne R D, et al. ROBUST: First report of phase III randomized study of lenalidomide/R-CHOP (R2-CHOP) vs placebo/R-CHOP in previously untreated ABC-type diffuse large B-cell lymphoma. Hematol Oncol. 2019; 37(52):36-37.
Wilson W H, Sin-Ho J, Pitcher B N, et al. Phase Ill Randomized Study of R-CHOP Versus DA-EPOCH-R and Molecular Analysis of Untreated Diffuse Large B-Cell Lymphoma: CALGB/Alliance 50303. Blood. 2016; 128(22):469-469.
Witzig T E, Reeder C B, LaPlant B R, et al. A phase II trial of the oral mTOR inhibitor everolimus in relapsed aggressive lymphoma. Leukemia. 2011; 25(2):341-347.
Woyach J A, Awan F, Flinn I W, et al. A phase 1 trial of the Fc-engineered CD19 antibody XmAb5574 (MOR00208) demonstrates safety and preliminary efficacy in relapsed CLL. Blood. 2014; 124(24):3553-3560.

### Appendix A

ECOG Performance Status Scale (Oken, 1982)

| Grade | Description |
|---|---|
| 0 | Fully active; able to carry on all pre-disease performance |
| | without restriction. |
| 1 | Restricted in physically strenuous activity |
| | but ambulatory and able to carry |
| | out work of a light or sedentary nature (e.g., light |
| | housework or office work). |
| 2 | Ambulatory and capable of all self-care but unable to carry |
| | out any work activities. Up and about >50% of waking hours. |
| 3 | Capable of only limited self-care; confined to a bed |
| | or chair >50% of waking hours. |
| 4 | Completely disabled. Cannot carry on any self-care. |
| | Totally confined to bed or chair. |
| 5 | Dead |

### Appendix B

Ann Arbor Staging

| Grade | Description |
|---|---|
| Stage I | Involvement of a single lymph node region (I) or of a |
| | single extralymphatic organ or site (IE)^{a} |
| Stage II | Involvement of two or more lymph node |
| | regions or lymphatic structures on |
| | the same side of the diaphragm alone (II) |
| | or with involvement of limited, |
| | contiguous extralymphatic organ or tissue (IIE) |
| Stage III | Involvement of lymph node regions |
| | on both sides of the diaphragm (III), |
| | which may include the spleen (IIIS) |
| | or limited, contiguous extralymphatic |
| | organ or site (IIIE), or both (IIIES) |
| Stage IV^{b} | Diffuse or disseminated foci of involvement of one |
| | or more extralymphatic organs or tissues, with or |
| | without associated lymphatic involvement |

Note:
All cases are subclassified to indicate the absence (A) or presence (B) of the systemic B symptoms of significant unexplained fever (>38° C.), night sweats, or unexplained weight loss exceeding 10% of body weight during the 6 months prior to diagnosis.
^{a}The designation “E” generally refers to extranodal contiguous extension (i.e., proximal or contiguous extranodal disease) that can be encompassed within an irradiation field
appropriate for nodal disease of the same anatomic extent. A single extralymphatic site as the only site of disease should be classified as IE, rather than Stage IV.
^{b}Involvement of bone marrow at screening will always qualify for Ann Arbor Stage IV and should be recorded as extranodal involvement.
Adapted from:
Carbone PP, Kaplan HS, Musshoff K, et al. Report of the committee on Hodgkin's disease staging classification. Cancer Res. 1971; 31:1860-1861.
Lister TA, Crowther D, Sutcliffe SB, et al. Report of a committee convened to discuss the evaluation and staging of patients with Hodgkin's disease: Cotswolds Meeting. J Clin Oncol. 1989; 7:1630-1636.

### Appendix C

| International Prognostic Index | |
|---|---|
| IPI Risk Factor | |
| Ann Arbor Stage III or IV | |
| Age >60 years | |
| Serum LDH >1 × ULN | |
| ECOG Performance status ≥2 | |
| Extranodal involvement ≥2¹ | |
| IPI Risk Groups | Number of IPI Risk Factors |
| Low | 0 or 1 |
| Low-Intermediate | 2 |
| High-Intermediate | 3 |
| High | 4 or 5 |

¹Extranodal involvement per Cheson 2014 can include sites that have focal uptake by PET-CT (e.g. spleen, liver, bone, thyroid, cutaneous, gastrointestinal (GI), bone, kidneys, pleural or pericardial effusions, ascities).
ECOG = Eastern Cooperative Oncology Group; FDG = fluorodeoxyglucose; IPI = International Prognostic Index; PET = positron emission tomography; ULN = upper limit of normal.
Adapted from:
Shipp MA, Harrington DP, Anderson JR, et al. A predictive model for aggressive non-Hodgkin's lymphoma. N Engl J Med. 1993; 329:987-94.

### Appendix D

### Lugano Response Criteria for Malignant Lymphoma (Cheson, 2014)

### Target and Non-Target Lesions

Up to six of the largest target nodes, nodal masses, or other lymphomatous lesions that are measurable in two diameters should be identified from different body regions representative of the patient's overall disease burden and include mediastinal and retroperitoneal disease, if involved. At baseline, a measurable node must be greater than 15 mm in longest diameter (LDi). Measurable extranodal disease may be included in the six representative, measured lesions. At baseline, measurable extranodal lesions should be greater than 10 mm LDi.

All other lesions (including nodal, extranodal, and assessable disease) should be followed as nonmeasured disease as non-target lesions (e.g. cutaneous, GI, bone, spleen, liver, kidneys, pleural or pericardial effusions, ascites, bone, bone marrow).

### Split Lesions and Confluent Lesions

Lesions may split or may become confluent over time. In the case of split lesions, the individual product of the perpendicular diameters (PPDs) of the nodes should be summed together to represent the PPD of the split lesion; this PPD is added to the sum of the PPDs of the remaining lesions to measure response. If subsequent growth of any or all of these discrete nodes occurs, the nadir of each individual node is used to determine progression. In the case of confluent lesions, the PPD of the confluent mass should be compared with the sum of the PPDs of the individual nodes, with more than 50% increase in PPD of the confluent mass compared with the sum of individual nodes necessary to indicate progressive disease. The LDi and smallest diameter (SDi) are no longer needed to determine progression.

| | | Lymph node | | | | |
|---|---|---|---|---|---|---|
| | | and extra | | | | |
| | | lymphatic | Non target | Liver and | | |
| Response | Imaging | sites | lesions | spleen | Bone marrow | New lesion |
| CR | PET | Score of 1, 2 | Not applicable | Not | No evidence | None |
| | | or 3^{a} with our | | applicable | of FDG-avid | |
| | | without a | | | disease in | |
| | | residual mass | | | marrow | |
| | | on 5PS^{b} | | | | |
| | CT | Target | Absent | Regress to | Normal by | None |
| | | nodes/nodal | | normal | morphology; | |
| | | masses must | | | if | |
| | | regress to ≤1.5 | | | intermediate, | |
| | | cm in LDi. | | | IHC negative | |
| | | No extra | | | | |
| | | lymphatic site | | | | |
| | | of disease | | | | |
| PR | PET | Score 4 or 5^{b} | Not applicable | Not | Residual | None |
| | | with reduced | | applicable | uptake higher | |
| | | uptake | | | than uptake in | |
| | | compared | | | normal marrow | |
| | | with baseline | | | but reduced | |
| | | and residual | | | compared with | |
| | | mass(es) of | | | baseline (diffuse | |
| | | any size | | | uptake | |
| | | At interim, | | | compatible with | |
| | | these findings | | | reactive changes | |
| | | suggest | | | from | |
| | | responding | | | chemotherapy | |
| | | disease | | | allowed). If there | |
| | | At end of | | | are persistent | |
| | | treatment, these | | | focal changes in | |
| | | findings | | | the marrow in the | |
| | | indicate | | | context of a | |
| | | residual disease | | | nodal response, | |
| | | | | | consideration | |
| | | | | | should be given | |
| | | | | | to further | |
| | | | | | evaluation with | |
| | | | | | MRI or biopsy or | |
| | | | | | an interval scan | |
| | CT | ≥50% | Abnormal/normal, | Spleen must | Not applicable | None |
| | | decrease in | regressed, but | have | | |
| | | SPD of up to 6 | no increase | regressed by | | |
| | | target | | >50% in | | |
| | | measurable | | length | | |
| | | nodes and | | beyond | | |
| | | extranodal | | normal | | |
| | | sites | | | | |
| | | When a lesion is | | | | |
| | | too small to | | | | |
| | | measure on CT, | | | | |
| | | assign 5 mm × 5 | | | | |
| | | mm as the | | | | |
| | | default value | | | | |
| | | When no longer | | | | |
| | | visible, 0 × 0 | | | | |
| | | mm | | | | |
| | | For a node >5 | | | | |
| | | mm × 5 mm, but | | | | |
| | | smaller than | | | | |
| | | normal, | | | | |
| | | use actual | | | | |
| | | measurement for | | | | |
| | | calculation | | | | |
| SD | PET | Score 4 or 5^{b} | Not applicable | Not | No change | None |
| | | with no | | applicable | from baseline | |
| | | significant | | | | |
| | | change in FDG | | | | |
| | | uptake from | | | | |
| | | baseline at | | | | |
| | | interim or end | | | | |
| | | of treatment | | | | |
| | CT | <50% | No increase | No increase | | |
| | | decrease from | consistent with | consistent | Not applicable | None |
| | | baseline in | progression | with | | |
| | | SPD of up to 6 | | progression | | |
| | | dominant, | | | | |
| | | measurable | | | | |
| | | nodes and | | | | |
| | | extranodal | | | | |
| | | sites; no | | | | |
| | | criteria for | | | | |
| | | progressive | | | | |
| | | disease are met | | | | |
| PD | PET | Score 4 or 5^{b} | Not applicable | Not | New or | New FDG- |
| | | with an | | applicable | recurrent | avid foci |
| | | increase in | | | FDG-avid foci | consistent |
| | | intensity of | | | | with |
| | | uptake from | | | | lymphoma |
| | | baseline and/or | | | | rather than |
| | | New FDG- | | | | another |
| | | avid foci | | | | etiology |
| | | consistent with | | | | (e.g., |
| | | lymphoma at | | | | infection, |
| | | interim or end- | | | | inflammation); |
| | | of-treatment | | | | if uncertain |
| | | assessment | | | | regarding |
| | | | | | | etiology of |
| | | | | | | new lesions, |
| | | | | | | biopsy or |
| | | | | | | interval scan |
| | | | | | | may be |
| | | | | | | considered |
| | CT | An individual | New or clear | New or | New or | Regrowth |
| | | node/lesion | progression of | recurrent | recurrent | of |
| | | must be | preexisting non- | splenomegaly | involvement | previously |
| | | abnormal with: | target lesions | | | resolved |
| | | LDi >1.5 cm | | | | lesions |
| | | and | | | | A new node |
| | | Increase by | | | | >1.5 cm in |
| | | ≥50% from PPD | | | | any axis |
| | | nadir and | | | | A new |
| | | An increase in | | | | extranodal |
| | | LDi or SDi | | | | site |
| | | from nadir | | | | >1.0 cm in |
| | | 0.5 cm for | | | | any axis; if |
| | | lesions ≤2 cm | | | | <1.0 cm in |
| | | 1.0 cm for | | | | any axis, its |
| | | lesions >2 cm | | | | presence |
| | | In the setting | | | | must be |
| | | of | | | | unequivocal |
| | | splenomegaly | | | | and must be |
| | | (>13 cm), the | | | | attributable |
| | | splenic length | | | | to lymphoma |
| | | must increase | | | | Assessable |
| | | by >50% of | | | | disease of |
| | | the extent of | | | | any size |
| | | its prior | | | | unequivocally |
| | | increase | | | | attributable |
| | | beyond | | | | to |
| | | baseline (e.g., | | | | lymphoma |
| | | a 15-cm spleen | | | | |
| | | must increase | | | | |
| | | to >16 cm). If | | | | |
| | | no prior | | | | |
| | | splenomegaly, | | | | |
| | | must increase | | | | |
| | | by at least 2 | | | | |
| | | cm from | | | | |
| | | baseline. | | | | |

5PS = 5-point scale;
CT = computed tomography;
FDG = fluorodeoxyglucose;
IHC = immunohistochemistry;
LDi = longest transverse diameter of a lesion;
MRI = magnetic resonance imaging;
PET = positron emission tomography;
PPD = cross product of the LDi and perpendicular diameter;
SDi = shortest axis perpendicular to the LDi;
SPD = sum of the product of the perpendicular diameters for multiple lesions.
^{a}A score of 3 in many patients indicates a good prognosis with standard treatment, especially if at the time of an interim scan. However, in trials involving PET where de-escalation is investigated, it may be preferable to consider a score of 3 as inadequate response (to avoid undertreatment). Measured dominant lesions: Up to six of the largest dominant nodes, nodal masses, and extranodal lesions selected to be clearly measurable in two diameters. Nodes should preferably be from disparate regions of the body and should include, where applicable, mediastinal and retroperitoneal areas. Non-nodal lesions include those in solid organs (e.g., liver, spleen, kidneys, lungs), gastrointestinal involvement, cutaneous lesions, or those noted on palpation. Non-measured lesions: Any disease not selected as measured; dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (e.g., GI tract, liver, bone marrow), FDG uptake may be greater than in the mediastinum with complete metabolic response, but should be no higher than surrounding normal physiologic uptake (e.g., with marrow activation as a result of chemotherapy or myeloid growth factors).
^{b}PET 5PS: 1 = no uptake above background; 2 = uptake ≤ mediastinum; 3 = uptake > mediastinum but ≤ liver; 4 = uptake moderately > liver; 5 = uptake markedly higher than liver and/or new lesions;
X = new areas of uptake unlikely to be related to lymphoma.

1-2. (canceled)

3. The method according to claim 24 comprising administering to the patient in at least one 21-day cycle a combination of:
an anti-CD19 antibody on day 1, day 8, and day 15 of the 21-day cycle;
rituximab on day 1 of the 21-day cycle;
cyclophosphamide on day 1 of the 21-day cycle;
doxorubicin on day 1 of the 21-day cycle;
vincristine on day 1 of the 21-day cycle; and
prednisone or prednisolone on each of days 1 to 5 of the 21-day cycle.

4. The method according to claim 3 comprising administering to the patient at least three 21-day cycles of the combination.

5. The method according to claim 3 comprising administering to the patient at least six 21-day cycles of the combination.

6. The method according to claim 24 comprising administering to the patient a combination of:
an anti-CD19 antibody;
rituximab in a 375 mg/m² dose;
cyclophosphamide in a 750 mg/m² dose;
doxorubicin in a 50 mg/m² dose;
vincristine in a 1.4 to 2.0 mg/m² dose; and
prednisone or prednisolone in a 100 mg dose.

7. The method according to claim 5 comprising administering to the patient in at least one 21-day cycle a combination of:
an anti-CD19 antibody on day 1, day 8, and day 15 of the 21-day cycle;
rituximab in a 375 mg/m² dose on day 1 of the 21-day cycle;
cyclophosphamide in a 750 mg/m² dose on day 1 of the 21-day cycle;
doxorubicin in a 50 mg/m² dose on day 1 of the 21-day cycle;
vincristine in a 1.4 to 2.0 mg/m² dose on day 1 of the 21-day cycle; and
prednisone or prednisolone in a 100 mg dose on each of days 1 to 5 of the 21-day cycle.

8. The method according to claim 7 comprising administering to the patient at least three 21-day cycles of the combination.

9. The method according to claim 7 comprising administering to the patient at least six 21-day cycles of the combination.

10. The method according to claim 24 wherein the anti-CD19 antibody is administered in a body weight dose of 8 mg/kg to 40 mg/kg.

11. The method according to claim 10 wherein the anti-CD19 antibody is administered in a body weight dose of 12 mg/kg.

12. The method according to claim 24 wherein the pharmaceutical combination further comprises lenalidomide.

13. The method according to claim 12 wherein lenalidomide is administered in a 25 mg dose.

14. The method according to claim 12 wherein lenalidomide is administered to the patient in at least one 21-day cycle in a 25 mg dose on each of days 1 to 10 of the 21-day cycle.

15. The method according to claim 24 further comprising the administration of granulocyte colony stimulating factor (G-CSF) or pegylated G-CSF.

16. The method according to claim 24, wherein administration of the combination leads to a complete response (CR) in the patient.

17. The method according to claim 24, wherein the anti-CD19 antibody comprises a variable heavy (VH) domain comprising VH complementarity determining region (CDR)1, VH CDR2, and VH CDR3, wherein:
the VH CDR1 comprises the amino acid sequence SYVMH (SEQ ID NO:1);
the VH CDR2 comprises the amino acid sequence NPYNDG (SEQ ID NO:2); and
the VH CDR3 comprises the amino acid sequence GTYYYGTRVFDY (SEQ ID NO:3); and
wherein the antibody comprises a variable light (VL) domain comprising VL CDR1, VL CDR2, and VL CDR3, wherein:
the VL CDR1 comprises the amino acid sequence RSSKSLQNVNGNTYLY (SEQ ID NO:4);
the VL CDR2 comprises the amino acid sequence RMSNLNS (SEQ ID NO:5); and
the VL CDR3 comprises the amino acid sequence MQHLEYPIT (SEQ ID NO:6).

18. The method according to claim 17, wherein the VH domain comprises the amino acid sequence EVQLVESGGGLVKPGGSLKLSCAASGYTFTSYVMHWVRQAPGKGLEWIGYINPYNDGTK YNEKFQGRVTISSDKSISTAYMELSSLRSEDTAMYYCARGTYYYGTRVFDYWGQGTLVT VSS (SEQ ID NO:7) and the VL domain comprises the amino acid sequence
(SEQ ID NO:8)
DIVMTQSPATLSLSPGERATLSCRSSKSLQNVNGNTYLYWFQQKPGQSP
QLLIYRMSNLNSGVPDRFSGSGSGTEFTLTISSLEPEDFAVYYCMQHLE
YPITFGAGTKLEIK.

19. The method according to claim 18, wherein the anti-CD19 antibody comprises a heavy chain region of
(SEQ ID NO: 11)
EVQLVESGGGLVKPGGSLKLSCAASGYTFTSYVMHWVRQAPGKGLEWI
GYINPYNDGTKYNEKFQGRVTISSDKSISTAYMELSSLRSEDTAMYYC
ARGTYYYGTRVFDYWGQGTLVTVSSASTKGPSVFPLAPSSKSTSGGTA
ALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLG
GPDVFLFPPKPKDTLMISRTPEVTCVVVDVSHEDPEVQFNWYVDGVEV
HNAKTKPREEQFNSTFRVVSVLTVVHQDWLNGKEYKCKVSNKALPAPE
EKTISKTKGQPREPQVYTLPPSREEMTKNQVSLTCLVKGFYPSDIAVE
WESNGQPENNYKTTPPMLDSDGSFFLYSKLTVDKSRWQQGNVFSCSVM
HEALHNHYTQKSLSLSPGK
and a light chain region of
(SEQ ID NO: 12)
DIVMTQSPATLSLSPGERATLSCRSSKSLQNVNGNTYLYWFQQKPGQS
PQLLIYRMSNLNSGVPDRFSGSGSGTEFTLTISSLEPEDFAVYYCMQH
LEYPITFGAGTKLEIKRTVAAPSVFIFPPSDEQLKSGTASVVCLLNNF
YPREAKVQWKVDNALQSGNSQESVTEQDSKDSTYSLSSTLTLSKADYE
KHKVYACEVTHQGLSSPVTKSFNRGEC.

20. The method according to claim 24, wherein the patient has previously untreated DLBCL.

21. The method according to claim 24, wherein the patient has an International Prognostic Index (IPI) status of 2-5, 3-5, 4-5, 3-4, 3, 4, or 5 prior to starting the administering.

22. The method according to claim 24, wherein the patient has Stage III or Stage IV DLBCL prior to starting the administering.

23. The method according to claim 24, wherein the anti-CD19 antibody is tafasitamab.

24. A method of treating a patient with diffuse large B-cell lymphoma (DLBCL) comprising administering to the patient a pharmaceutical combination comprising a therapeutic amount of:
an anti-CD19 antibody; and
rituximab, cyclophosphamide, doxorubicin, vincristine, and prednisone or prednisolone (R-CHOP).

25. A method of treating a non-Hodgkin lymphoma, chronic lymphocytic leukemia, or acute lymphoblastic leukemia in a human subject in need thereof, the method comprising administering to the human subject a therapeutically effective amount of an antibody that binds to human CD19, lenalidomide, and rituximab.

26. The method of claim 25, wherein the antibody comprises a variable heavy (VH) domain comprising VH complementarity determining region (CDR)1, VH CDR2, and VH CDR3, wherein:
the VH CDR1 comprises the amino acid sequence SYVMH (SEQ ID NO:1);
the VH CDR2 comprises the amino acid sequence NPYNDG (SEQ ID NO:2); and
the VH CDR3 comprises the amino acid sequence GTYYYGTRVFDY (SEQ ID NO:3); and
wherein the antibody comprises a variable light (VL) domain comprising VL CDR1, VL CDR2, and VL CDR3, wherein:
the VL CDR1 comprises the amino acid sequence RSSKSLQNVNGNTYLY (SEQ ID NO:4);
the VL CDR2 comprises the amino acid sequence RMSNLNS (SEQ ID NO:5); and
the VL CDR3 comprises the amino acid sequence MQHLEYPIT (SEQ ID NO:6).

27. The method of claim claim 26, wherein the VH domain comprises the amino acid sequence EVQLVESGGGLVKPGGSLKLSCAASGYTFTSYVMHWVRQAPGKGLEWIGYINPYNDGTK YNEKFQGRVTISSDKSISTAYMELSSLRSEDTAMYYCARGTYYYGTRVFDYWGQGTLVT VSS (SEQ ID NO:7) and the VL domain comprises the amino acid sequence
(SEQ ID NO: 8)
DIVMTQSPATLSLSPGERATLSCRSSKSLQNVNGNTYLYWFQQKPGQS
PQLLIYRMSNLNSGVPDRFSGSGSGTEFTLTISSLEPEDFAVYYCMQH
LEYPITFGAGTKLEIK.

28. The method of claim 27, wherein the antibody comprises a heavy chain region of EVQLVESGGGLVKPGGSLKLSCAASGYTFTSYVMHWVRQAPGKGLEWIGYINPYNDGTK YNEKFQGRVTISSDKSISTAYMELSSLRSEDTAMYYCARGTYYYGTRVFDYWGQGTLVT VSSASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPCPAP ELLGGPDVFLFPPKPKDTLMISRTPEVTCVVVDVSHEDPEVQFNWYVDGVEVHNAKTKP REEQFNSTFRVVSVLTVVHQDWLNGKEYKCKVSNKALPAPEEKTISKTKGQPREPQVYT LPPSREEMTKNQVSLTCLVKGFYPSDIAVEWESNGQPENNYKTTPPMLDSDGSFFLYSK LTVDKSRWQQGNVFSCSVMHEALHNHYTQKSLSLSPGK (SEQ ID NO: 11) and a light chain region of
(SEQ ID NO: 12)
DIVMTQSPATLSLSPGERATLSCRSSKSLQNVNGNTYLYWFQQKPGQS
PQLLIYRMSNLNSGVPDRFSGSGSGTEFTLTISSLEPEDFAVYYCMQH
LEYPITFGAGTKLEIKRTVAAPSVFIFPPSDEQLKSGTASVVCLLNNF
YPREAKVQWKVDNALQSGNSQESVTEQDSKDSTYSLSSTLTLSKADYE
KHKVYACEVTHQGLSSPVTKSFNRGEC.

29. The method of claim 25, wherein the human subject has a non-Hodgkin lymphoma.

30. The method of claim 29, wherein the non-Hodgkin lymphoma is follicular lymphoma.

31. The method of claim 30, wherein the follicular lymphoma is relapsed/refractory follicular lymphoma.

32. The method of claim 30, wherein the follicular lymphoma is histologically confirmed Grade 1, 2, or 3a follicular lymphoma.

33. The method of claim 29, wherein the non-Hodgkin lymphoma is marginal zone lymphoma.

34. The method of claim 33, wherein the marginal zone lymphoma is relapsed/refractory marginal zone lymphoma.

35. The method of claim 33, wherein the marginal zone lymphoma is histologically confirmed nodal marginal zone lymphoma, splenic marginal zone lymphoma, or extranodal marginal zone lymphoma of the mucosa-associated lymphoid tissue.

36. The method of claim 29, wherein the non-Hodgkin lymphoma is diffuse large B-cell lymphoma.

37. The method of claim 36, wherein the diffuse large B-cell lymphoma is relapsed/refractory diffuse large B-cell lymphoma.

38. The method of claim 29, wherein the non-Hodgkin lymphoma is small lymphocytic lymphoma.

39. The method of claim 29, wherein the non-Hodgkin lymphoma is mucosa-associated lymphoid tissue lymphoma.

40. The method of claim 29, wherein the non-Hodgkin lymphoma is Burkitt's lymphoma.

41. The method of claim 29, wherein the non-Hodgkin lymphoma is mantle cell lymphoma.

42. The method of claim 25, wherein the human subject has chronic lymphocytic leukemia.

43. The method of claim 25, wherein the human subject has acute lymphoblastic leukemia.

44. The method of claim 25, wherein the antibody is administered intravenously.

45. The method of claim 25, wherein the antibody is administered intravenously at a dose of 12 mg/kg.

46. The method of claim 25, wherein the antibody is administered intravenously at least once every two weeks at a dose of 12 mg/kg.

47. The method of claim 25, wherein the antibody is administered intravenously at a dose of 12 mg/kg according to the following schedule:
on days 1, 8, 15, and 22 of a first 28-day cycle;
on days 1, 8, 15, and 22 of a second 28-day cycle;
on days 1, 8, 15, and 22 of a third 28-day cycle; and
on days 1 and 15 of a fourth 28-day cycle and on days 1 and 15 of further 28-day cycles thereafter.

48. The method of claim 25, wherein rituximab is administered intravenously.

49. The method of claim 25, wherein rituximab is administered intravenously at a dose of 375 mg/m².

50. The method of claim 25, wherein rituximab is administered intravenously at a dose of 375 mg/m² according to the following schedule:
on days 1, 8, 15, and 22 of a first 28-day cycle; and
on day 1 of a second 28-day cycle and on day 1 of further 28-day cycles thereafter.

51. The method of claim 25, wherein lenalidomide is administered orally.

52. The method of claim 25, wherein lenalidomide is administered orally at a dose of 20 mg.

53. The method of claim 25, wherein lenalidomide is administered orally at a dose of 20 mg on days 1-21 of repeated 28-day cycles.

54-76. (canceled)

77. A method for treatment of a patient with DLBCL comprising administering an anti-CD19 antibody.

78-106. (canceled)

107. The method according to claim 25 wherein the anti-CD19 antibody is administered in a body weight dose of 8 mg/kg to 40 mg/kg.
